(19) United States
(12) Patent Application Publication
(10) Pub. No.: US 2011/0243992 A1
(43) Pub. Date: Oct. 6, 2011

(54) METHODS OF ENHANCING THE IMMUNOGENICITY OF MYCOBACTERIA AND COMPOSITIONS FOR THE TREATMENT OF CANCER, TUBERCULOSIS, AND FIBROSING LUNG DISEASES

(75) Inventor: Douglas S. Kernodle, Brentwood, TN (US)

(73) Assignee: Vanderbilt University, Nashville, TN (US)

(21) Appl. No.: 13/061,315

(22) PCT Filed: Aug. 31, 2009
(86) PCT No.: PCT/US09/55550

§ 371 Date: Jun. 22, 2011

Related U.S. Application Data
(60) Provisional application No. 61/092,942, filed on Aug. 29, 2008.

(51) Int. Cl. A61K 39/04; C12N 15/74; C12N 1/21; A61P 35/00; A61P 37/04; A61P 31/06; A61P 11/00; A61P 31/04

(52) U.S. Cl. 424/248.1; 435/471; 435/252.3

(57) ABSTRACT

Whole-cell vaccines and methods for enhancing the immunogenicity of cellular microorganisms for use in producing protective immune responses in vertebrate hosts subsequently exposed to pathogenic bacteria or for use as vectors to express exogenous antigens and induce responses against other infectious agents or cancer cells. The present invention involves an additional method of enhancing antigen presentation by intracellular bacteria in a manner that improves vaccine efficacy. After identifying an enzyme that has an anti-apoptotic effect upon host cells infected by an intracellular microbe, the activity of the enzyme produced by the intracellular microbe is reduced by expressing a mutant copy of the enzyme, thereby modifying the microbe so that it increases immunogenicity.

### CROSS-REFERENCE TO RELATED APPLICATIONS

This application claims the benefit of 61/092,942, filed Aug. 29, 2008, which application is incorporated herein by reference.

This invention was made with government support under SERCEB (Southeastern Regional Center for Excellence in Research in Biodefense and Emerging Infectious Diseases) NIH Grant U54A1057157 and some of the work involved the use of research facilities in Department of Veteran's Affairs Medical Centers. The U.S. Government has certain rights in this invention.

### BACKGROUND OF THE INVENTION

1. Field of the Invention

The present invention relates to the field of vaccination including the induction of strong immune responses and the prevention and treatment of infectious diseases and cancer. Specifically, the present invention relates to methods for enhancing the immunogenicity of a bacterium by reducing the activity of superoxide dismutase, thioredoxin, thioredoxin reductase, glutamine synthase, and other anti-apoptotic enzymes. This can be achieved by expressing dominant-negative mutants of enzymes, by inactivation of genes that regulate the expression or secretion of the targeted enzyme, by allelic inactivation, and by other methods. It further relates to methods for producing a safe and effective vaccine and methods for enhancing an effective immune response in host animals subsequently exposed to infection by bacterial pathogens, for example, Mycobacterium tuberculosis. The immunogenic vaccines constructed by using these methods can also be vectors for expressing exogenous antigens and used to induce an immune response against unrelated infectious agents and cancer. Finally, it relates to methods for diminishing the immune evasive capacity and the iron-scavenging capacity of mycobacteria in vivo. These methods can be applied to enhance the potency of mycobacteria administered as immunotherapy in cancer, to treat persons with latent tuberculosis infection or active tuberculosis, and to treat other mycobacterial infections including those associated with fibrosing lung diseases.

2. Background

Carcinoma of the bladder accounts for about 2% of all solid tumors in the United States with more than 50,000 new cases being diagnosed each year. The peak prevalence of bladder cancer is in individuals 60-70 years old and several etiologic factors have been implicated including smoking and exposure to industrial chemicals. Pathologically, carcinoma of the bladder is categorized by grade (usually I-IV) and by depth of malignancy (either superficial, invasive, or metastatic bladder cancer). Superficial bladder cancer, which is confined to the bladder epithelium, usually presents as papillary tumors (stages Ta or T1) or carcinoma-in-situ. Diagnosis of bladder cancer is by cytoscopy and biopsy. At the time of diagnosis, about 70% of patients have only superficial disease, 25% have locally invasive disease, and 5% already have distant metatasis.

Superficial bladder cancer is treated with transurethal resection (TURBT) and/or fulguration. Cytoscopy is usually reserved for those tumors which cannot be resected transurethally. After TURBT, 50% of patients remain disease free; however the other half experience multiple recurrences with about 10% developing invasive or metastatic disease within 3-4 years. Superficial recurrences are treated with TURBT, often followed by intravesical chemotherapy to prevent or delay any additional recurrence. Patients who are considered at high risk for recurrence after the initial TURBT (e.g. high grade, multi-focal, and/or large tumors), or those with concurrent CIS are frequently given intravesical adjunct therapy as prophylaxis against recurrence. Intravesical BCG administration is the treatment of choice for this adjunct therapy.

TICE® BCG was licensed in the United States in 1989 for the treatment of carcinoma-in-situ but not for papillary Ta or T1 lesions. To obtain licensure for the treatment of carcinoma-in-situ with the TICE substrain, the sponsor submitted efficacy data on 119 evaluable patients with biopsy proven CIS. The data was derived from six uncontrolled phase II trials. No controlled phase III trials were done. The primary endpoint evaluated was the incidence of complete responses (CR). The initial response based on a two year follow-up was 75.6%. Aftera median duration of follow-up of 47 months, there were 45 CRs, resulting in an overall long-term response of 38%. At this time 85 patients (71%) were alive, 18 patients (15%) had died of bladder cancer and 13 (12%) had died of other causes. The advisory committee noted that historical data obtained prior to the use of intravesical BCG showed that 34% of CIS patients died of this disease in five years.

The current application discloses methods for reducing the activity of an anti-apoptotic microbial enzyme in Mycobacterium. Also disclosed are modified bacteria made in accordance with the disclosed methods that have enhanced anti-cancer effects.

Thirty patients with malignant pleural mesothelioma have been treated with BCG vaccine immunotherapy. There was an improved survival rate, compared with patients treated symptomatically only. See Webster et al., Immunotherapy with BCG vaccine in 30 cases of mesothelioma. S Afr Med J. 1982 Feb. 20; 61(8):277-8.

The mechanism by which BCG is beneficial in the treatment of bladder appears to involve BCG's ability to recruit and activate immune cells, particularly cells involved in the innate immune response to infection such as natural killer (NK) cells and polymorphonuclear leukocytes (PMNs). There is evidence that BCG attaches to fibronectin in the tumor milieu and then enters into epithelial cells including the malignant cells. As the NK cells, PMNs, and other immune cells respond to the BCG bacilli, they exert a cytotoxic effect on the tumor cells.

The original use of BCG was against tuberculosis (TB). TB is an infectious disease caused by Mycobacterium tuberculosis. A third of the world's population, about 2 billion people, are infected with M. tuberculosis and from this enormous reservoir of infection there are about 9 million new cases of active TB annually. Persons who are infected but without active disease are considered to have latent TB infection and they remain at risk for the development of active TB for the rest of their lives. BCG (Bacillus Calmette-Guerin) was derived from a virulent strain of M. bovis and was first administered as a vaccine to prevent TB in the 1920s. BCG is effective in preventing disseminated forms of TB including TB meningitis and miliary TB in early childhood and the administration of BCG to about 100 million newborns globally each years prevents about 40,000 cases of disseminated TB annually. Unfortunately BCG is much less effective in preventing the pulmonary, contagious form of TB that causes most of the global burden of disease. Furthermore, although immunotherapy with extracts of mycobacteria have been used an adjunctive therapy in persons with active TB, the benefit has been modest. In addition, BCG is of no benefit when administered to persons with latent TB infection and there is no alternative vaccine or immunotherapy shown to be of value in such persons.

Finally, there is growing evidence that infection with Mycobacterium species contribute to the pathogenesis of other lung diseases, including sarcoidosis. In some persons, sarcoidosis leads to progressive lung fibrosis.

### SUMMARY OF THE INVENTION

The present invention involves a method of modifying a Mycobacterium to enhance the recruitment and activation of innate immune cells. Some of the innate immune cells, in particular NK cells and PMNs, release granules when activated by the modified Mycobacterium strain to kill bystander cells including tumor cells. Furthermore, the enhancement of innate immune responses leads to enhanced antigen presentation and the development of stronger adaptive immune responses involving CD4+ lymphocytes in a manner that induces immune memory and improves vaccine efficacy. The enhanced memory immune responses can be directed towards exogenous antigens, including tumor antigens, inserted into the Mycobacterium as well as antigens intrinsic to the Mycobacterium. Modifying a Mycobacterium to express a pro-apoptotic phenotype is provided, as are modifications that reduce the expression of transferrin receptors and the cellular uptake of iron by macrophages that can otherwise lead to cell necrosis instead of apoptosis.

Also, as the induction of strong CD8+ T-cell responses has generally been difficult to achieve with current vaccination strategies, the present modified microbes provide a very effective way to access this arm of the immune system. The microbe can be further altered by adding exogenous DNA encoding immunodominant antigens from other pathogenic microbes including viruses, bacteria, protozoa, and fungi or with DNA encoding cancer antigens, and then used to vaccinate a host animal. Therefore, the present attenuated bacterium can be used as a vaccine delivery vehicle to present antigens for processing by MHC Class I and MHC Class II pathways. And because of strong co-stimulatory signals induced by microbial components in the vaccine vector that interact with Toll-like receptors on the host cell, this directs the host immune system to react against the exogenous antigen rather than develop immune tolerance. Furthermore, the simultaneous presentation of antigens by MHC Class I and MHC Class II pathways by dendritic cells facilitates the development of CD4 “help” for CD8 cytotoxic T-lymphocyte (CTL) responses, thereby overcoming limitations of antigen presentation by current vectors that have been designed to access either exogenous (e.g., many bacterial vectors, phagosome-associated) or endogenous (e.g., many viral vectors, cytoplasm and proteasome-associated) pathways of antigen presentation.

The present invention also provides a method of targeting Mycobacterium inside a host, reducing the ability of the Mycobacterium to induce the expression of transferrin receptors and the cellular uptake of iron by macrophages. Immunizing an uninfected or infected host with antioxidant enzymes of the Mycobacterium, in particular immunization with the iron co-factored superoxide dismutase (SodA), generates the production of antibodies and cellular immune responses that reduce the activity of the Mycobacterium enzyme. Such immunization can be performed prior to administering BCG therapeutically to persons with bladder cancer or other malignancies. Immunization can also be given to persons in whom BCG is used as an adjuvant together with a cancer vaccine, as a way to enhance the potency of the adjuvant effects from the live BCG bacilli. Furthermore, a person with latent TB infection, a person with active TB, or a person with fibrosing lung disease caused by a Mycobacterium can be immunized with the enzyme. Subsequently, the Mycobacterium that infects the host has diminished potential to promote the uptake of iron by macrophages and cause damage to lung tissue that manifests either as granulomatous lung pathology, the development of lung cavities, or fibrosing lung disease.

### BRIEF DESCRIPTION OF THE DRAWINGS

FIG. 1 shows figures of the iron co-factored superoxide dismutase of M. tuberculosis/BCG (SodA). (A) SodA monomer showing positions of deleted amino acids in the present SodA mutants. Other deletions, additions, and/or substitutions can be used to produce additional dominant-negative SodA mutants. (B) shows SodA tetramer with each rectangle indicating the position of two active site iron ions. The arrows identify active-site iron and E54 positions for the same monomer. The figure was downloaded from the National Center for Biotechnology Information (NCBI) web server (www.ncbi.nlm.nih.gov/entrez/query.fcgi?db=Structure&itool=toolbar) and modified to illustrate features.

FIG. 2 provides a map (A) and features (B) of mycobacterial chromosomal integration vector pMP399, and a map (C) and features (D) of plasmid vector pMP349 that expresses mutant SodA ΔH28ΔH76 in BCG. The name for the gene encoding iron co-factored superoxide dismutase in M. tuberculosis/BCG is sodA. It is expressed behind an inducible aceA(icl) promoter. The E. coli origin of replication (oriE) allows the plasmid to replicate in E. coli. The apramycin resistance gene (aacC41) and vectors pMP399 and pMP349 was developed by Consaul and Pavelka. The apramycin resistance gene can be replaced by a different antibiotic resistance gene or the vector can contain a biosynthetic gene that complements amino acid auxotrophy in the bacterial strain, thereby allowing growth on media lacking the essential factor (e.g., the amino acid) to be used as a selectable marker for identification of successful recombinants.

FIG. 3 shows SOD activity in supernatants and lysates of BCG that expresses mutant SodA (ΔH28ΔH76) compared to SOD activity of the parent BCG strain. (A) and (B) show results from two separate experiments. The assay is performed using serial 2-fold dilutions of supernatant and lysate and monitoring the amount of reduced cytochrome C at a fixed time point. A unit of SOD activity inhibits cytochrome C reduction by 50% (of the maximal measured inhibition). The dilution that inhibits cytochrome C reduction by 50% (IC50 value) for each preparation is indicated by arrows. SodA is secreted by BCG and thus the SOD activity of BCG supernatant is greater than the SOD activity of BCG lysate.

FIG. 4 shows SOD activity in supernatants and lysates of BCG that expresses mutant SodA (ΔE54) compared to SOD activity of the parent BCG strain.

FIG. 5 shows comparative vaccine efficacy of BCG versus SD-BCG-AS-SOD. The SD-BCG (SodA-diminished BCG) strains used in these experiments were constructed using antisense techniques (see WO 02/062298 entitled “Pro-apoptotic bacterial vaccines to enhance cellular immune responses,” incorporated herein by reference for its teaching of antisense reduction in SOD activity), and exhibit about 1% of the SOD activity of the parent BCG strains. C57Bl/6 mice were vaccinated IV with BCG or SD-BCG-AS-SOD, rested for 7 months, and then challenged by aerosol with 30 cfu of an acriflavin-R mutant of the virulent Erdman strain of M. tuberculosis. At 14 wk post-challenge, unvaccinated and BCG-vaccinated mice displayed focal areas of densely cellular parenchymal lung inflammation (representative section shown in A, ×2 and ×10). In contrast, SD-BCG-vaccinated mice had less densely cellular areas of lung involvement (B, ×2 and ×10). Higher power views of B (C) show foamy cells with nuclear fragments indicative of ingested apoptotic debris in an alveolus (left panel) and multinucleated giant cells (right panel). At the time of final harvest at six months post-challenge, Erdman cfu counts were lower in recipients of SD-BCG compared to recipients of BCG (D). The line within the box plot represents the median, the edges of the box indicate 25th and 75th percentiles, and the whiskers represent 10th and 90th percentiles. The difference between groups was statistically significant (P=0.04, two-sample t-test). Also at this time, the final mean weights of mice in each group were 28.3 and 31.0 gms, BCG [8 survivors from original 12 mice, 4 euthanized from skin problems] and SD-BCG [10 survivors] respectively, P=0.04, two-sample t-test. Thus, reducing SodA production by BCG enhanced its efficacy as a vaccine.

FIG. 6 shows that vaccination with SD-BCG-AS-SOD alters recall T-cell responses in the lungs of mice post-aerosol challenge with virulent M. tuberculosis. Mice were vaccinated with 2×10⁶ cfu subQ with either BCG, SD-BCG-AS-SOD, or phosphate-buffered saline (unvaccinated), rested for 100 days, and then challenged with 300 cfu of Erdman by aerosol. Values represent the number of cells expressing the indicated surface antigens (left column) recovered from the right lung of mice at 4, 10, and 18 days post-challenge. Both lungs were harvested from control mice. Each value represents the mean of 4 mice, except that 3 mice were used for the control values. The BCG-vaccinated group includes mice that received either BCG or C-BCG. Recipients of SD-BCG exhibited greater numbers of CD44+/CD45RB^{high} cells by day 4 post-infection. These cells were larger than other T-cell populations by forward scatter and can represent T-cells undergoing clonal expansion. By day 18, larger numbers of terminally-differentiated CD4+ effector T-cells (CD44+/CD45RB^{neg}) were observed in recipients of SD-BCG than BCG. *P=0.02; ¶P<0.05, BCG versus SD-BCG, two-sample t-test.

FIG. 7 shows accelerated formation of Ghon lesions in mice vaccinated with SD-BCG-AS-SOD after aerosol challenge with 300 cfu of an acriflavin-R mutant of the virulent Erdman strain of M. tuberculosis. Low (×2) and mid (×20) power photomicrographs of left lungs at day 18 post-challenge are shown. Between day 10 and day 18 post-challenge, SD-BCG-vaccinated developed numerous small focal aggregates of cells in the lung parenchyma (right panels). Such changes between day 10 and day 18 were less apparent in BCG-vaccinated mice and not observed in unvaccinated mice. The small focal cell collections in SD-BCG mice differed in appearance from the expanding areas of granulomatous inflammation in BCG-vaccinated mice, showing more large mononuclear cells with pale cytoplasm and early foamy changes, often containing nuclear fragments indicative of apoptotic cell debris.

FIG. 8 shows the map (A) and features (B) of the vector that was used to inactivate sigH on the chromosome of BCG and construct SIG-BCG (BCGΔsigH).

FIG. 9 shows lung cfu counts at 6 months post aerosol challenge. Mice were rested for 100 days following subQ vaccination with BCG or BCGΔsigH and then challenged with 300 cfu of an acriflavin-R mutant of the virulent Erdman strain of M. tuberculosis. The line within the box plot represents the median, the edges of the box indicate 25th and 75th percentiles, and the whiskers represent 10th and 90th percentiles. The difference between groups was statistically significant (P=0.019, two-sample T-test.).

FIG. 10 shows photomicrographs of lung sections of mice vaccinated with placebo (saline), BCG, or BCGΔsigH at 6 months post-challenge with 300 cfu of an acriflavin-R mutant of the virulent Erdman strain of M. tuberculosis. Lungs from two mice in each group were inflated with 10% buffered formalin and paraffin-embedded. Three low-power photomicrographs covering about 80% of the lung tissue sections shown on the microscope slide are displayed and show less diseased lungs in the mice vaccinated with BCGΔsigH. Boxes indicates regions shown under higher-power magnification in FIG. 11.

FIG. 11 shows the formation and evolution of Ghon lesions (arrows) at 22 days, 2 mo., and 6 mo post-aerosol challenge of mice with 300 cfu of an acriflavin-R mutant of the virulent Erdman strain of M. tuberculosis. Mice were vaccinated with placebo (saline), BCG, or BCGΔsigH subcutaneously and rested for 100 days before aerosol challenge. Ghon lesions develop earlier in BCGΔsigH-vaccinated mice and evolve with less granulomatous inflammation, thereby resulting in minimal lung damage. In contrast, areas of dense parenchymal infiltration by lymphocytes and macrophages develop in the lungs of unvaccinated and BCG-vaccinated mice. The 6-month photomicrographs correspond to the boxed regions in FIG. 10.

FIG. 12 illustrates sequential steps in immune activation and shows how microbial anti-oxidants can interfere with the activation of the immune response in its early stages. Reducing the activity of microbial anti-oxidants favors apoptosis and other immune functions during vaccination. This leads to strong memory T-cell responses and enhanced protection.

FIG. 13 shows a strategy for combining gene deletions and dominant-negative mutations in multiple genes to yield progressively more potent pro-apoptotic BCG strains to use as vaccines against tuberculosis and as vectors for expressing exogenous antigens. The pro-apoptotic vaccine strains are constructed using a “generation” approach where the 1^{st} generation involves modification of BCG to include a single gene inactivation or dominant-negative mutant enzyme expression, the 2^{nd} generation combines two modifications, the 3^{rd} generation combines three modifications, and the 4^{th} generation combines four modifications.

FIG. 14 shows SOD activity in supernatants and lysates of SIG-BCG and SAD-SIG-BCG. SIG-BCG (also referred to as “sigH-deleted BCG”, or “BCGΔsigH”) is designated BCGdSigH in this figure. SAD-SIG-BCG (also referred to as “BCGΔsigH [mut sodA]” is designated BCGdSigH H28H76 (panels A and B) or BCGdSigH E54 (panel C), depending upon which dominant-negative mutant was tested. “supe” is an abbreviation for supernatant. The assay is performed using serial 2-fold dilutions of supernatant and lysate and monitoring the amount of reduced cytochrome C at a fixed time point. A unit of SOD activity inhibits cytochrome C reduction by 50% (of the maximal measured inhibition). The dilution that inhibits cytochrome C reduction by 50% (IC50 value) for each preparation is indicated by arrows.

FIG. 15 shows Southern hybridization results that verify the construction of DD-BCG (“double-deletion BCG”), as referred to as “BCGΔsigHΔsecA2.” Chromosomal DNA from four isolates was digested with DraIII, applied to lanes 1-4, and then hybridized with gene probes. The gene probes were directed against secA2, sigH, and hygR (the gene encoding a hygromycin resistance cassette used in the insertional inactivation of sigH). The hygromycin-resistance gene (hygR) had an internal restriction site predicted to yield 2.92 and 1.67 kb fragments when a double-crossover event between the vector and chromosome had eliminated sigH and thus provided additional assurance of success (beyond the absence of a sigH band). The sequence of events in the construction of DD-BCG included the following steps: Starting with the BCG Tice strain (Lane 1) the secA2 gene in BCG Tice was inactivated by using methods previously used to inactivate secA2 in a virulent M. tuberculosis strain [Braunstein, M. et al, 2002; Braunstein, M. et al, 2003, incorporated herein by reference for its teaching of methods to inactivate secA2], thereby producing BCGΔsecA2 (Lane 2). The allelic inactivation vector shown in FIG. 8 was used to inactivate sigH in BCG to yield BCGΔsigH (Lane 3) and also to delete sigH in BCGΔsecA2, thereby yielding BCGΔsigHΔsecA2 (Lane 4, DD-BCG).

FIG. 16 shows SOD activity in lysates of sigH-secA2-deleted BCG (BCGΔsigHΔsecA2, also referred to as double-deletion BCG [“DD-BCG”]) and DD-BCG strains that express mutant SodA (ΔE54) or mutant SodA (ΔH28ΔH76), which are also referred to as 3D-BCG-mutSodA(ΔE54), and 3D-BCG-mutSodA(ΔH28ΔH76). These examples of 3D-BCG strains involve the pMP399-derived vectors and have a mut sodA inserted into the chromosome (of DD-BCG). Panel (A) shows results for supernatants and lysates. Supernatants exhibit less SOD activity than lysates because of the inactivation of secA2, which encodes the secretion channel for SodA and catalase. Panels B-D show SOD activity results from three separate experiments involving lysates prepared on different days using independent cultures of each isolate. The assay is performed using serial 2-fold dilutions of supernatant and lysate and monitoring the amount of reduced cytochrome C at a fixed time point. A unit of SOD activity inhibits cytochrome C reduction by 50% (of the maximal measured inhibition). The dilution where that inhibits cytochrome C reduction by 50% (IC50 value) for each preparation is indicated by arrows.

FIG. 17 shows SDS-PAGE and Western hybridization of lysates of DD-BCG (lane 3), 3D-BCG-mutSodA(ΔE54) (lane 4), and 3D-BCG-mutSodA(ΔH28ΔH76) (lane 5). These examples of 3D-BCG strains have a mut sodA inserted into the chromosome of DD-BCG. The Western hybridization gel shows comparable amounts of SodA in lysates of DD-BCG and two 3D-BCG constructs. Undiluted lysates for PAGE and Western were prepared as described in the methods for the examples (below). BSA=bovine serum albumin, a prominent component in broth media. The E. coli SOD (lane 2) does not react with the antibody against M. tuberculosis SodA. The undiluted lysates applied to these gels are the same as the lysates used in the SOD activity assays shown in FIG. 16D. Thus, although the SOD activity is markedly reduced by expressing of the mutant sodA genes, the amount of SodA protein as shown on SDS-PAGE and Western appear comparable. These data are consistent with a “dominant-negative” effect rendered by expression of the mutant SodA.

FIG. 18 shows a figure of the glnA1 hexameric ring comprised of six monomers. The figure was downloaded from the NCBI web server (www.ncbi.nlm.nih.gov/entrez/query.fcgi?db=Structure&itool=toolbar) and modified to illustrate features. GlnA1 monomers form dodecamers comprising two hexameric rings. The squares indicate the position of the active-sites, which are located between adjacent monomers and comprised of manganese ions and catalytic loops from the adjacent monomers. The deleted amino acids in the mutant glnA1 include an aspartic acid at amino acid 54 and glutamic acid at amino acid 335 (GlnA1ΔD54ΔE335), which are in the active-site and correspond to D50 and G327 of the Salmonella glutamine synthase.

FIG. 19 provides a map (A) and features (B) of the plasmid vector pHV203-mut glnA1 ΔD54ΔE335 that expresses the dominant-negative mutant glnA1 in BCG.

FIG. 20 provides a map (A) and features (B) of plasmid vector pMP349, and a map (C) and features (D) of the mycobacterial chromosomal integration vector pMP399 that express mutant SodA ΔH28ΔH76 and mutant glnA1 ΔD54ΔE335 in BCG.

FIG. 21 shows an example of exogenous antigen expression by pro-apoptotic BCG. SDS-PAGE (upper panel) and Western hybridization (lower panel) with an anti-BLS antibody verify expression of recombinant Brucella lumazine synthase (rBLS) by DD-BCG, which is seen as an 18-kDa band in lane 5 under inducing conditions. rBLS was cloned behind an aceA (id) promoter. BSA=bovine serum albumin, which was present in broth cultures, other bands in lanes 4-6 represent proteins of DD-BCG or rBLS. Lanes 5 and 6 represent DD-BCGrBLS grown under conditions that induce (+, addition of acetate) and suppress (−, addition of succinate) the aceA (id) promoter and thus the production of rBLS.

FIG. 22 shows the map (A) and features (B) of the vector used to inactivate thioredoxin (trxC) and thioredoxin reductase (trxB2) on the chromosome of BCG.

FIG. 23 shows the map (A) and features (B) of the vector to replace the wild-type alleles for thioredoxin (trxC) and thioredoxin reductase (trxB2) on the chromosome of BCG with mutant alleles in which six amino acids of each enzyme that correspond to the active sites have been eliminated.

FIG. 24 shows the map (A) and features (B) of the vector used to inactivate sigE on the chromosome of BCG.

FIG. 25 shows reduced glutamine synthetase activity in modified BCG strains that express the ΔD54ΔE335 dominant-negative mutant of glnA1 described in Example 8. Panel (A) shows SDS-PAGE (upper) and Western hybridization blot (lower) of lysates (L) of BCG, 3D-BCG, and 4D-BCG as well as partially-purified lysates following ammonium sulfate (AS) precipitation. 4D-BCG was constructed by electroporating the plasmid pHV203-mutGlnA1ΔD54ΔE335 (Table 1) into 3D-BCG. The GlnA1 monomer migrates between the 50- and 37-kDa markers and shows comparable amounts of GlnA1 produced by BCG, 3D-BCG, and 4D-BCG. Panel (B) shows the glutamine synthase activity in the AS-treated lysates of 3D-BCG and 4D-BCG, representing the same AS preparations shown in (A). The reaction was followed spectrophotometrically by monitoring absorbance over time. 3D-BCG AS lysate: ∘, undiluted; □, 2-fold dilution; Δ, 4-fold dilution; ⋄, 8-fold dilution. 4D-BCG AS lysate: , undiluted; ▪, 2-fold dilution. Despite comparable amounts of GlnA1 protein as shown in (A), enzyme activity was barely detected in 4D-BCG with the undiluted 4D-BCG prep exhibiting activity comparable to an 8-fold dilution of the 3D-BCG prep. This demonstrates that expression of the ΔD54ΔE335 monomer exerts a dominant-negative effect upon enzyme activity. Panel (C) shows a repeat enzyme activity assay involving two culture preparations of the pHV203-mutGlnA1ΔD54ΔE335 version of 4D-BCG. In addition, the pMP399 version of 4D-BCG was constructed by electroporating the chromosomal integration vector pMP399-mutSodAΔH28ΔH76,mutGlnA1 ΔD54ΔE335 (Table 1) into DD-BCG. The pMP399 version of 4D-BCG does not achieve quite as potent a reduction of glutamine synthetase activity as does the pHV203 version, probably related to a copy number effect from expressing the D54ΔE335 GlnA1 mutant from the chromosome (i.e., single copy) versus a multicopy plasmid, respectively.

FIG. 26 shows the production of IFN-γ and IL-2 by CD4+ T-cells following vaccination with BCG and paBCG vaccines. (A) The percent of CD4+ T-cells from the spleens of C57Bl/6 mice that produce INF-γ and IL-2 were plotted against days after IV vaccination with BCG, DD-BCG, 3D-BCG, and 4D-BCG. Each data point in each panel represents a single mouse and displays the % of CD4+ splenocytes that produce INF-γ or IL-2 after overnight restimulation on BCG-infected macrophages minus the % cells producing INF-γ or IL-2 after restimulation on uninfected macrophages. The shaded area shows the mean value±2 standard deviations for splenocytes from PBS-vaccinated mice analyzed in a similar fashion, indicating very low background with the IFN-γ assays and relatively higher background with IL-2. (B) Summary of the % INF-γ+ and % IL-2+ CD4+ T-cells from BCG-versus paBCG-vaccinated mice, using only the subset of mice that had an IFN-γ value of ≧0.5%. This eliminated results from mice harvested before the onset of the primary T-cell response, as well as results from recipients of the more advanced 3D- and 4D-BCG vaccines in which cytokine production quickly declined to almost baseline values following primary proliferation (panel A) but then was rapidly recalled during reinfection (see FIG. 27). The dot-plots show median, 25-75 percentile (box), and 10-90 percentile (whiskers) values. Whereas BCG typically induced more IFN-γ production, the IL-2 values were significantly higher in mice vaccinated with the paBCG vaccines, P=0.0024.

FIG. 27 shows T-cell responses to vaccination with BCG, DD-BCG, and 3D-BCG at day 25 and day 31 post-vaccination. BCG-specific cytokine production by splenocytes from mice vaccinated 25 days and 31 earlier. The vaccine dose was 5×10⁵ cfu administered intravenously. Splenocytes were incubated overnight on IFN-γ-treated uninfected bone marrow-derived macrophages (BMDMs) or IFN-γ-treated BCG-infected BMDMs. T-cells were then evaluated by flow cytometry for production of INF-gamma and IL-2 by intracellular cytokine staining techniques. The percent of IFN-γ-producing and IL-2-producing CD4+ and CD8+ T-cells is shown within the boxed areas. Background cytokine production was determined from the unstimulated values (uninfected macrophages). Note: In contrast to the data shown in FIG. 26A, the % values shown here represent % of the total CD4 population without subtracting the baseline value (uninfected BMDM) from the BCG-infected BMDM value after restimulation. Raw data from this plot were converted for incorporation into FIG. 26A. For example, the data points at 0.73% (0.86−0.13) and 1.47% (1.52−0.05) for IFN-γ production at days 25 and 31, respectively, and −0.03% (0.15−0.18) and 0.18% (0.28−0.10) for IL-2, respectively, come from this experiment.

FIG. 28 shows secondary (recall) T-cell responses in BCG-vaccinated mice and 3DBCG-vaccinated mice at 5 days post-intratracheal challenge with 4×10⁷ cfu of BCG. Mice were vaccinated subQ with 5×10⁵ cfu of the vaccine strain three months earlier and from 4-8 weeks post-vaccination were treated with INH and rifampin to eliminate the vaccine strain. Antigen-specific production of IFN-γ was 1.35% (1.58−0.23) and 0.85% (2.09−1.24%) in two BCG-vaccinated mice versus 7.88% (8.09−0.21) and 3.85% (4.09−0.024) in two 3DBCG-vaccinated mice. Antigen-specific co-production of IFN-γ and IL-2 was 0.29% (0.29−0.0) and 0.10% (0.15−0.03) in the BCG mice versus 2.01% (2.02−0.01) and 1.09% (1.15−0.06) in 3DBCG mice.

FIG. 29 shows the relative expression of mRNA, as determined by RT-PCR, from the spleens of mice 72 hours after IV vaccination with BCG, 3dBCG, and control (broth diluent). The inoculum was 1.5×10⁷ CFU. The mean value of the control group is set at 1.0 and the relative expression of six mice in each vaccination arm are plotted. * signifies P<0.05, ** signifies P<0.001. The results indicate that BCG upregulates the expression of TfR (transferrin receptors) whereas 3dBCG does not. This difference does not appear to be due to differences in the expression of IL-4 or IFN-γ, which were comparable for BCG and 3dBCG.

FIG. 30 shows a diagram of a possible mechanism by which SodA promotes iron over-loading of macrophages that results in the formation of toxic oxygen radicals and the damage of lung tissue. SodA, the iron co-factored superoxide dismutase of Mycobacterium species including M. tuberculosis, M. bovis, and M. bovis BCG, dismutates O₂⁻ to form H₂O₂. These oxidants exhibit opposite effects on the mRNA binding activity of IRP1 (iron regulatory protein 1). Similar to iron depletion, H₂O₂ activates IRP1 whereas O₂⁻ interferes with the ability of IRP1 to bind to the iron-regulatory element of TfR mRNA to promote its stability and facilitate translation. This results in the overloading of macrophages with iron such that a greater proportion of host-generated H₂O₂ is converted into toxic oxygen intermediates instead of either being inactivated or converted into long-lived oxidants such as taurine chloramine with low capacity for tissue damage. The overloading of macrophages with iron also makes them less capable of producing or responding to cytokines including IFN-γ. This causes a general impairment of the innate response to vaccination. By reducing the activity and secretion of SodA, the modified bacterium induces relatively greater early immune activation, including greater activation and recruitment of immune cells important to a tumoricidal response such as NK cells and polymorphonuclear neutrophils.

FIG. 31 shows H&E-stained lung tissue from C57Bl/6 mice after intratracheal inoculation of 5×10⁶ cfu of Mycobacterium vaccae expressing recombinant SodA (MVrSodA). Low-power and mid-power magnifications of the lung tissue are shown at 1 month, 2 months, and 3 months post-inoculation (A). A higher power view of the lungs at 2 months post-inoculation shows multiple dark cells representing hemosiderin (iron)-laden macrophages (B)

FIG. 32 shows fibrosis of parenchymal lung tissue at 16 weeks following intratracheal inoculation of a C57Bl/6 mouse with 5×10⁶ cfu of Mycobacterium vaccae expressing recombinant SodA (MVrSodA). Collagen is stained by the trichrome blue stain and demonstrates diffuse fibrosis.

### DETAILED DESCRIPTION OF THE INVENTION

It must be noted that, as used in the specification and the appended claims, the singular forms “a,” “an,” and “the” include plural referents unless the context clearly dictates otherwise. Thus, for example, reference to “an enzyme” includes multiple copies of the enzyme and can also include more than one particular species of enzyme.

A method of modifying a microbe to enhance the immunogenicity of the microbe is provided, comprising reducing the activity of an anti-apoptotic enzyme produced by the microbe by overexpressing a dominant-negative mutant enzyme and/or inactivation of a regulatory gene that controls the production of anti-apoptotic enzymes, whereby the bacterium has enhanced immunogenicity in a subject. When the anti-apoptotic enzyme with reduced activity is SOD, particularly the secreted iron co-factored SOD (called SodA) of Mycobacterium species, including M. tuberculosis, M. bovis, and M. bovis BCG, there is an additional advantage conferred by a reduction in the expression of transferrin receptors (TfR) on the infected host cell such that the iron content of the host cell is reduced, thereby leading to greater responsiveness to immune signaling and diminished necrosis. The dominant-negative mutant of SodA or glutamine synthase is a mutant enzyme that when expressed by the bacterium reduces the total SOD or glutamine synthase activity of the bacterium. The modified bacteria can also contain a mutation in a regulatory gene that reduces its activity or inactivates it. As used herein, a mutation that causes reduced activity (an activity reducing mutation) encompasses an inactivating mutation. Thus, also provided is an intracellular microbe, modified to reduce the activity of an anti-apoptotic enzyme of the microbe. The invention also provides a method of modifying an attenuated microbe to enhance the immunogenicity of the attenuated microbe, comprising reducing the activity of an anti-apoptotic enzyme produced by the attenuated microbe by overexpressing a dominant-negative mutant enzyme and/or inactivation of a regulatory gene that controls the production of anti-apoptotic enzymes, whereby the attenuated bacterium has enhanced immunogenicity in a subject. Thus, also provided is an attenuated intracellular microbe, further modified to reduce the activity of an anti-apoptotic enzyme of the microbe.

The invention further provides a method of modifying the enzymatic activity of a bacterium that has been administered or can be administered as immunotherapy to a subject, e.g., BCG, or a bacterium, e.g., Mycobacterium tuberculosis, that is already causing infection in a subject, comprising immunizing the mammalian subject with the microbial enzyme to induce antibodies or cellular immune responses that diminish the in vivo activity of the microbial enzyme, whereby the bacterium has enhanced immunogenicity and when the enzyme is SodA, reduced capacity to promote the expression of transferrin receptors in a subject. Thus, also provided are the enzymes, formulated to induce immune responses that reduce the activity of an anti-apoptotic enzyme of the microbe.

The microbe can be any Mycobacterium species described herein. Examples of species of Mycobacterium include, but are not limited to, M. tuberculosis, M. bovis, M. bovis strain BCG including BCG substrains, M. avium, M. intracellulare, M. africanum, M. kansasii, M. marinum, M. ulcerans and M. paratuberculosis. The construction of SOD-diminished mutants of these species can achieve both attenuation and confer the pro-apoptotic quality that enhances the development of strong cellular immune responses in a manner analogous to the present SOD-diminished BCG vaccine, as secretion of iron-manganese SOD is a common and distinctive attribute of many of the pathogenic species of mycobacteria. Accordingly, SOD-diminished vaccines of these other mycobacterial species can be highly effective vaccine strains and because of the immune-enhancing characteristics of the mycobacterial cell wall, useful as adjuvants and immunotherapy in cancer.

Thus, a specific embodiment of the invention provides a live vaccine against tuberculosis, derived by diminishing the activity of iron-manganese superoxide dismutase (SOD) in a strain of M. tuberculosis or BCG by overexpressing a dominant-negative mutant SOD enzyme.

The invention provides a method of making a microbial vaccine, comprising reducing the activity of an anti-apoptotic enzyme produced by the microbe, wherein the reduction in the activity of the anti-apoptotic enzyme attenuates the microbe, whereby a microbial vaccine is produced.

The invention provides a method of making a microbial vaccine, comprising reducing in an attenuated microbe the activity of an anti-apoptotic enzyme produced by the microbe, whereby a microbial vaccine is produced.

The present invention provides a composition comprising a microbe comprising an enzyme modified by the methods of the present invention. The composition can further comprise a pharmaceutically acceptable carrier or a suitable adjuvant. Such a composition can be used as a vaccine or as immunotherapy against infectious diseases, cancer, and fibrosing lung diseases.

The modified bacterium can include a dominant-negative mutant selected from the group consisting of a) SodA in which a deletion, insertion, and/or substitution of nucleotides in the naturally occurring nucleic acid encodes a molecule that reduces the SOD activity of the organism; and b) glutamine synthase in which a deletion, insertion, and/or substitution of nucleotides in the naturally occurring nucleic acid encodes a molecule that reduces the glutamine synthase activity of the organism. In one embodiment, the modified bacterium can be BCG. Thus, a BCG modified to express reduced SOD activity is provided.

The modified bacterium can comprise a further pro-apoptotic modification involving reducing the activity of other microbial enzymes including thioredoxin, thioredoxin reductase, glutamine synthetase, and other redox related enzymes such as glutathione reductase (glutaredoxin), other thioredoxin-like proteins, other thioredoxin reductase-like proteins, other glutaredoxin-like proteins, other thiol reductases, and other protein disulphide oxidoreductases. Specific examples of additional redox-related enzymes in mycobacteria include, but are not limited to, thiol peroxidase, the NAD(P)H quinone reductase Rv3303c (lpdA), and the whiB family of thioredoxin-like enzymes. Further pro-apoptotic modifications affect genes that influence either the production or secretion of the anti-apoptotic microbial enzyme and can comprise one or more modification selected from the group consisting of inactivation of SigH, inactivation of sigE, and inactivation of SecA2. Thus, a BCG modified to express reduced SOD activity and no SigH is provided. A BCG modified to express reduced SOD activity, no SigH and no sigE is provided. A BCG modified to express reduced SOD activity, no SigH, no sigE, and no SecA2 is also provided.

Specific examples of modified bacteria are described in the examples and Table 1. For example, the modified bacterium can comprise a mutant SodA having deletions of histidine at position 28 and histidine at position 76, a mutant SodA having a deletion of histidine at position 28 or a histidine at position 76, a mutant SodA having a deletion of glutamic acid at position 54, a mutant SodA having a deletion of glutamic acid at position 54 and the replacement of histidine with arginine at position 28. In further examples, the modified bacterium can comprise modifications selected from the group consisting of a mutant of SodA and inactivation of sigH; a mutant of SodA and inactivation of secA2; a mutant of SodA, inactivation of sigH and inactivation of secA2; and a mutant of SodA, a dominant-negative mutant of glnA1, inactivation of sigH and inactivation of secA2.

As further examples of the modified bacterium, the bacterium can comprise a mutation of glnA1 selected from the group consisting of deletions of aspartic acid at amino acid 54 and glutamic acid at amino acid 335; and a deletion of aspartic acid at amino acid 54 or a glutamic acid at amino acid 335. The bacterium with reduced glnA1 activity can further comprise inactivation of secA2. The bacterium with reduced glnA1 activity can further comprise a dominant-negative mutant of SodA. In the bacterium with reduced glnA1 activity and a dominant-negative mutant of SodA, the mutant SodA can comprise deletions of histidine at position 28 and histidine at position 76. The bacterium with reduced glnA1 activity can further comprise inactivation of sigH and inactivation of secA2. The bacterium with reduced glnA1 activity can further comprise a dominant-negative mutant of SodA and inactivation of sigH. In the bacterium with reduced glnA1 activity and a dominant-negative mutant of SodA, the dominant-negative mutant is a mutant SodA having a deletion of glutamic acid at position 54. In the bacterium with reduced glnA1 activity and a dominant-negative mutant of SodA, the dominant-negative mutant is a mutant SodA having deletions of histidine at position 28 and histidine at position 76. In the bacterium with reduced glnA1 activity activity and a dominant-negative mutant of SodA, the bacterium can further comprise a dominant-negative mutant of SodA and inactivation of secA2. Methods of making the bacteria described in the description, in Table 1, the examples and figures are provided.

The modified bacterium of the invention can comprise inactivation of sigH. The modified bacterium can comprise inactivation of sigH and inactivation of secA2.

The present invention additionally provides a method of producing an immune response in a subject by administering to the subject any of the compositions of this invention, including a composition comprising a pharmaceutically acceptable carrier and a microbe comprising an enzyme necessary for in vivo viability that has been modified according to the methods taught herein. The composition can further comprise a suitable adjuvant, as set forth herein. The subject can be a mammal and is preferably a human.

The present invention provides a method of preventing an infectious disease in a subject, comprising administering to the subject an effective amount of a composition of the present invention. In addition to preventing bacterial diseases, for example, tuberculosis, it is contemplated that the present invention can prevent infectious diseases of fungal, viral and protozoal etiology. The subject can be a mammal and preferably human.

It is contemplated that the above-described compositions of this invention can be administered to a subject or to a cell of a subject to impart a therapeutic benefit or immunity to prevent infection. Thus, the present invention further provides a method of producing an immune response in an immune cell of a subject, comprising contacting the cell with a composition of the present invention, comprising a microbe in which an enzyme necessary for in vivo viability has been modified by any of the methods taught herein. The cell can be in vivo or ex vivo and can be, but is not limited to, an MHC I-expressing antigen presenting cell, such as a dendritic cell, a macrophage or a monocyte. As used throughout, by a “subject” is meant an individual. Thus, the “subject” can include domesticated animals, such as cats, dogs, etc., livestock (e. g., cattle, horses, pigs, sheep, goats, etc.), laboratory animals (e. g., mouse, rabbit, rat, guinea pig, etc.) and birds. Preferably, the subject is a mammal such as a primate, and, more preferably, a human.

The invention, therefore, provides a method of enhancing the immunogenicity of an attenuated bacterium, comprising reducing the activity of an anti-apoptotic enzyme produced by the bacterium, whereby the bacterium has enhanced immunogenicity in a subject. The bacterium modified by reducing the activity of an anti-apoptotic enzyme can be selected from the group consisting of M. tuberculosis, M. bovis, M. avium, M. intracellulare, M. africanum, M. kansasii, M. marinum, M. ulcerans, M. paratuberculosis, and other Mycobacterium species.

Provided is a method for facilitating antigen presentation via construction of pro-apoptotic vaccines made by reducing the production of microbial anti-apoptotic enzymes including SOD, thioredoxin, thioredoxin reductase, glutamine synthetase, and other redox related enzymes such as glutathione reductase (glutaredoxin), other thioredoxin-like proteins, other thioredoxin reductase-like proteins, other glutaredoxin-like proteins, other thiol reductases, and other protein disulphide oxidoreductases. Many of these enzymes are highly conserved in all cellular life forms and many overlap or are identical to the enzymes that detoxify reactive oxygen intermediates due to the central role of reactive oxygen species (ROS) as a trigger for apoptosis. The decision to make pro-apoptotic vaccines relates to the capability of the enzyme from the intracellular pathogen to block apoptosis when the pathogen is within the host cell, as is the case with virulent strains of M. tuberculosis. For example, SodA produced by M. tuberculosis detoxifies superoxide (O₂⁻), which is an oxidant with pro-apoptotic biological effects that is produced by the phagocyte oxidase (NADPH oxidase) of immune cells. Accordingly, by reducing the activity of SodA and other microbial enzymes that inactivate the oxidants produced by host immune cells, one can simultaneously attenuate the microbe and enhance the presentation of its antigens, as dendritic and other immune cells process the apoptotic phagocytes (e.g., neutrophils, monocytes and/or macrophages) containing microbial antigens.

Some anti-apoptotic microbial enzymes can be eliminated without adversely affecting the ability to cultivate the microbe as a vaccine strain, and for such enzymes, traditional molecular genetic techniques including allelic inactivation can be used to construct the modified microbe. However, some enzymes are absolutely essential for the viability of the microbe, such that they cannot be eliminated entirely. For these enzymes, techniques of genetic manipulation by which mutants with a partial rather than complete reduction in the activity of the anti-apoptotic enzyme are constructed. Anti-sense RNA overexpression is described in WO 02/062298 as one such strategy for constructing mutant strains with partial phenotypes, and its utility as a tool to screen and identify which essential enzymes can be reduced to render a pro-apoptotic phenotype was also emphasized.

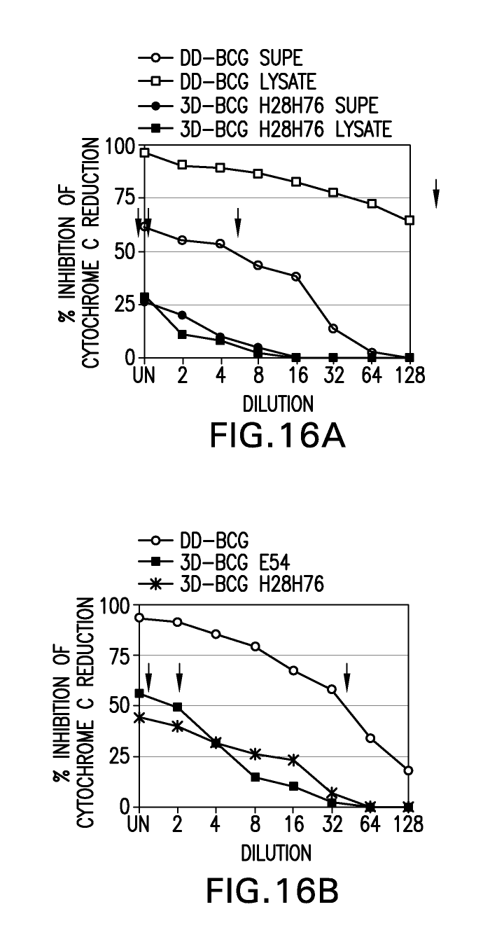

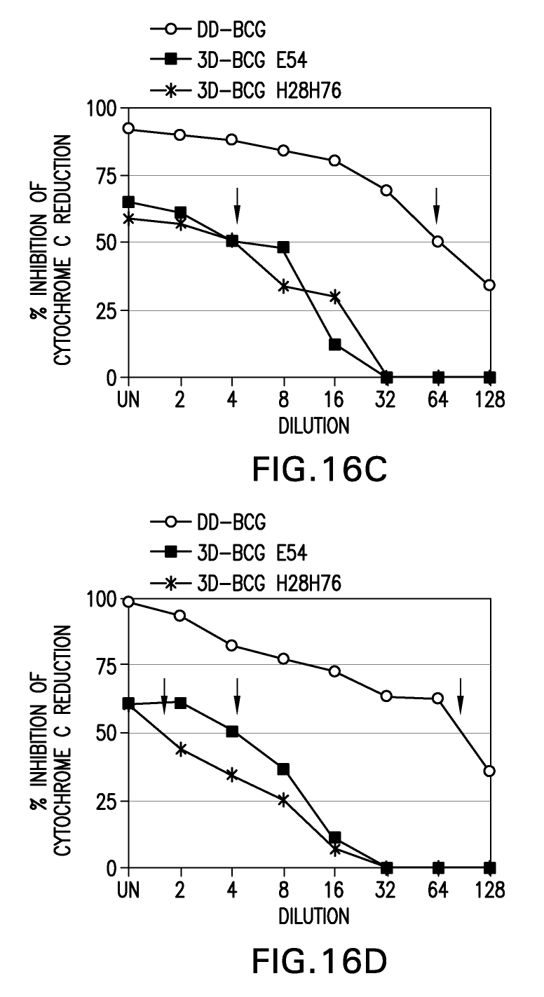

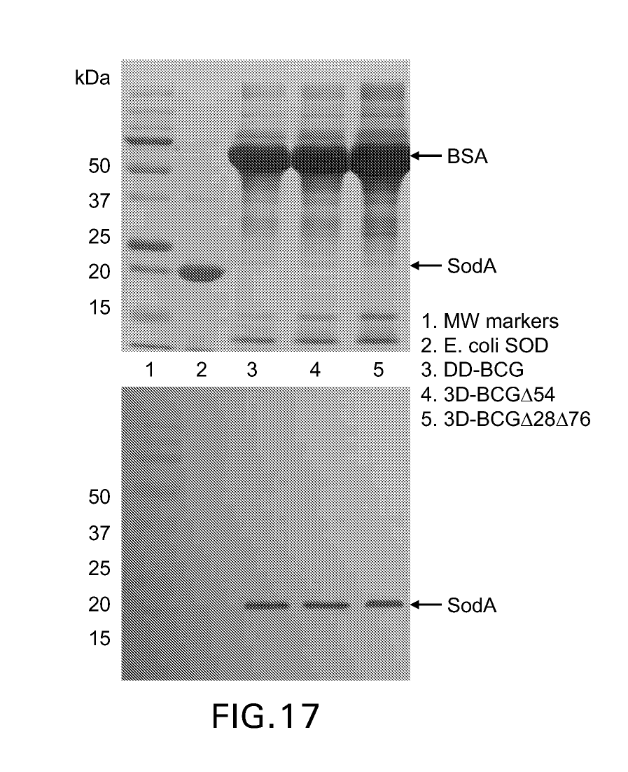

The current invention outlines three additional strategies for achieving a partial reduction in the activity of anti-apoptotic microbial enzymes. The first strategy involves the overexpression of dominant-negative mutants of the enzyme. The second strategy involves allelic inactivation of a regulatory gene that governs the expression of the anti-apoptotic enzyme. Both strategies represent additional methods for stably modifying a microbe to render a partial phenotype, whereby the microbe retains or increases immunogenicity but loses or reduces pathogenicity in a subject, comprising reducing but not eliminating an activity of an enzyme produced by the microbe, whereby reducing the activity of the enzyme attenuates the microbe or further attenuates the microbe. The third strategy is focused on inducing an immune response to the anti-apoptotic enzyme to interfere with the activity of the enzyme in vivo. This strategy can be achieved by vaccinating with bacteria expressing a dominant-negative mutant of the enzyme, or alternatively by vaccinating directly with the mutant enzyme. The dominant-negative mutants are immunogenic yet lack the immune-suppressive characteristics of the wild-type enzyme. For example, the dominant-negative mutants of SodA react with anti-SodA antibodies (FIG. 17) yet exhibit diminished SOD activity (FIG. 16). This method can be combined with the administration of a stably modified microbe with diminished activity of the enzyme, or alternatively can be combined with administration of the parent, unaltered microbe in a prime-boost strategy. For example, the host can first be vaccinated with a current parent BCG vaccine (e.g., BCG Danish 1331, BCG Tokyo 172) or a stably modified, pro-apoptotic BCG (paBCG) vaccine and subsequently vaccinated with a booster vaccine comprising paBCG or the dominant-negative mutant SodA enzyme. These vaccines can also be administered to persons previously infected with a pathogenic microbe, for example, M. tuberculosis, to lessen the pathologic consequences of infection.

Dominant-negative enzyme mutants can comprise either mutations that yield a modified enzyme with partial enzyme activity or mutations that yield an inert enzyme completely devoid of enzyme activity. As the effect of co-expressing the mutant enzyme in a cell that also expresses the wild-type enzyme is typically a reduction rather than complete elimination of the whole-cell enzymatic activity, this strategy can be directed against genes that are essential for the viability of the microbe.

The strategy of reducing the activity of anti-apoptotic enzymes by using dominant-negative techniques can be employed in wild-type bacterial strains as a means to make the strain partially- or fully-attenuated while increasing its immunogenicity. It can also be applied to strains that are already attenuated and/or current vaccine strains, for example, to enhance the immunogenicity of Bacillus Calmette-Guerin (BCG), the current vaccine for tuberculosis.

Examples of the constructs provided herein and examples of constructs used to make the present constructs are provided in Table 1.

The compositions of the present invention can be administered in vivo to a subject in need thereof by commonly employed methods for administering compositions in such a way to bring the composition in contact with the population of cells. The compositions of the present invention can be administered orally, parenterally, intramuscularly, transdermally, intradermally, percutaneously, subcutaneously, extracorporeally, topically or the like, although oral or parenteral administration are typically preferred. It can also be delivered by introduction into the circulation or into body cavities, by ingestion, or by inhalation. The vaccine strain is injected or otherwise delivered to the animal with a pharmaceutically acceptable liquid carrier, that is aqueous or partly aqueous, comprising pyrogen-free water, saline, or buffered solution. For example, an M. tuberculosis vaccine can be administered similar to methods used with US BCG Tice strain, percutaneously using a sterile multipuncture disk.

The methods and compositions using the modified, pro-apoptotic BCG (mBCG, paBCG) vaccines of this invention can be used to treat or prevent solid tumors selected from the group consisting of skin cancer, brain cancer, oropharyngeal cancer, breast cancer, lung cancer, esophageal cancer, stomach cancer, liver cancer, colon cancer, cancer of the biliary tract, pancreatic cancer, anal cancer, kidney cancer, prostate cancer, and sarcoma. The skin cancer can be melanoma or squamous cell; the brain cancer can be glioblastoma, astrocytoma or oligodendroglioma; the lung cancer can be primary tumor or metastasis of other tumors to lung; and the liver cancer can be primary tumor (hepatoma) or metastasis of other tumors to the liver. Veterinary cancers that can be treated or prevented by use of paBCG or other vaccine in combination with paBCG include equine sarcoids, bovine ocular squamous cell carcinoma, and bovine vulval carcinoma. It is understood that the disclosed methods, include, in one aspect, treating a cancer by administering a pro-apoptotic BCG to a subject, wherein the treating of a cancer comprises prolonging the survival of the subject with the cancer. It is further understood that the methods of prevention can also include methods of reducing the likelihood of cancer developing in a subject comprising administering paBCG to the subject.

It is further understood that treatment can comprise any positive change in the disease statuts of a subject suffering from the disease. For example, treating can comprise a 5%, 10%, 20%, 30%, 40%, 50%, 60%, 70%, 80%, 90%, 100% or any amount in between reduction in the symptoms or cause of a disease or condition, such as a cancer or tuberculosis. Thus, a treatment includes but is not limited to the complete ablation of a disease as well as more modest changes. Similarly, a treatment can comprise a delay in a negative outcome such as a prolonging of survival even if the subject eventually succumbs to the disease. Thus, a method comprising administering to a subject with a cancer a pro-apoptotic BCG to a subject, wherein the administration of the pro-apoptotic BCG prolongs to the subject the survival of the subject with the cancer is a treatment.

When administered as immunotherapy for cancer or carcinoma in situ to recruit and activate NK cells, PMNs, and other cells of the innate immune response, the composition can be administered directly into the tumor by injection via a needle. Visible lesions on the surface of the skin (e.g., melanoma) or mucous membranes (e.g., oral tumors, rectal tumors) can be visualized directly to determine the site of injection. Alternatively, the composition can be delivered with assistance of radiologic imaging, e.g., CT-guided placement of the needle within a tumor in the lung, liver, kidney, pancreas or other organ. Endoscopic techniques can also be used to administer the composition. For immunotherapy of bladder tumors, the composition can be administered directly via catheter into the bladder using methods similar to US BCG Tice strain.

When administered as an adjuvant to strengthen the immune responses induced by another vaccine, the compositions of the present invention are typically first mixed with the other vaccine preparation, for example, a vaccine comprising a recombinant cancer antigen. Then the combined formulation is administered parenterally to a subject.

The modified BCG (paBCG) vaccines constructed using this technology are superior to currently-available BCG vaccine for every cancer indication for which BCG is currently used including but not limited to immunotherapy against bladder cancer and melanoma in man, as an adjuvant combined with autologous colon cancer cells in man, and as immunotherapy for veterinary tumors. Furthermore, because of the superior ability of the paBCG vaccines to recruit and activate NK cells, CD8 T cells, and other immune cells as well as to induce the production of anti-tumor cytokines including IL-12 and IL-21, paBCG exhibits anti-tumor activity beyond the current indications. Some of the potential uses are discussed below.

### Cancer Vaccines/Adjuvants

The present methods and compositions using paBCG can be used to treat or prevent cancer, both as a vaccine and as an adjuvant for a cancer vaccine (e.g., autologous tumor cell vaccine or recombinant cancer antigen vaccine). Cancer vaccines are intended either to treat existing cancers (therapeutic vaccines) or to prevent the development of cancer (prophylactic vaccines). Both types of vaccines have are used to reduce the burden of cancer. Treatment or therapeutic vaccines are administered to cancer patients and are designed to strengthen the body's natural defenses against cancers that have already developed. These types of vaccines prevent the further growth of existing cancers, prevent the recurrence of treated cancers, or eliminate cancer cells not killed by prior treatments. Prevention or prophylactic vaccines, on the other hand, are administered to healthy individuals and are designed to target cancer-causing viruses and prevent viral infection.

At this time, two vaccines have been licensed by the U.S. Food and Drug Administration to prevent virus infections that can lead to cancer: the hepatitis B vaccine, which prevents infection with the hepatitis B virus, an infectious agent associated with some forms of liver cancer; and Gardasil™, which prevents infection with the two types of human papillomavirus (HPV)—HPV 16 and 18—that together cause 70 percent of cervical cancer cases worldwide. Gardasil also protects against infection with HPV types 6 and 11, which account for 90 percent of cases of genital warts.

Vaccines used to treat cancers take advantage of the fact that certain molecules on the surface of cancer cells are either unique or more abundant than those found on normal or non-cancerous cells. These molecules, either proteins or carbohydrates, act as antigens, meaning that they can stimulate the immune system to make a specific immune response. It is understood that when a vaccine containing cancer-specific antigens is injected into a patient, these antigens stimulate the immune system to attack cancer cells without harming normal cells.

Researchers have developed several strategies to stimulate an immune response against tumors. One is to identify unusual or unique cancer cell antigens that are rarely present on normal cells. Other techniques involve making the tumor-associated antigen more immunogenic, or more likely to cause an immune response, such as (a) altering its amino acid structure slightly, (b) placing the gene for the tumor antigen into a viral vector (a harmless virus that can be used as a vehicle to deliver genetic material to a targeted cell), and (c) adding genes for one or more immuno-stimulatory molecules into vectors along with the genes for the tumor antigen. Another technique is to attach something that is clearly foreign, known as an adjuvant, to tumor molecules. By using the adjuvant as a decoy, the immune system can be “tricked” into attacking both the antigen/adjuvant complex (the vaccine) and the patient's tumor.

The types of vaccines listed below represent various methods investigators have devised for presenting cancer antigens to the body's immune system. This list is not meant to be comprehensive.

### Antigen/Adjuvant Vaccines

Antigen vaccines were some of the first cancer vaccines investigated. Antigen vaccines commonly use specific protein fragments, or peptides, to stimulate the immune system to fight tumor cells. One or more cancer cell antigens are combined with a substance that causes an immune response, known as an adjuvant. A cancer patient is vaccinated with this mixture. Thus, the immune system, in responding to the antigen-carrying adjuvant, also responds to tumor cells that express that antigen.

### Whole Cell Tumor Vaccines

Taken either from the patient's own tumor (autologous) or tumor cells from one or more other patients (allogeneic), these whole cell vaccine preparations contain cancer antigens that are used to stimulate an immune response.

### Dendritic Cell (DC) Vaccines

Specialized white blood cells, known as dendritic cells (DCs), are taken from a patient's blood through a process called leukapheresis. In the laboratory, the DCs are stimulated with the patient's own cancer antigens, grown in petri dishes, and re-injected into the patient. Once injected, DC vaccines activate the immune system's T cells. Activation by DCs can cause T cells to multiply and attack tumor cells that express that antigen.

### Viral Vectors and DNA Vaccines

Viral vectors and DNA vaccines use the nucleic acid sequence of the tumor antigen to produce the cancer antigen proteins. The DNA containing the gene for a specific cancer antigen is manipulated in the laboratory so that it can be taken up and processed by immune cells called antigen-presenting cells (APCs). The APC cells then display part of the antigen together with another molecule on the cell surface. When these antigen-expressing APC cells are injected into a person, the immune system responds by attacking not only the APC cells, but also tumor cells containing the same antigen. Vector-based and DNA vaccines are attractive because they are easier to manufacture than some other vaccines.

### Idiotype Vaccines

Because antibodies contain proteins and carbohydrates, they can themselves act as antigens and induce an antibody response. Antibodies produced by certain cancer cells (i.e., B-cell lymphomas and myelomas), called idiotype antibodies, are unique to each patient and can be used to trigger an immune response in a manner similar to antigen vaccines.

Cancer cell antigens can be unique to individual tumors, shared by several tumor types, or expressed by the normal tissue from which a tumor grows. In 1991, the first human cancer antigen was discovered in the cells of a patient with metastatic melanoma, a potentially lethal form of skin cancer. The discovery led to a flurry of research to identify antigens for other cancers.

### Treatment Vaccines

Patient-specific vaccines use a patient's own tumor cells to generate a vaccine intended to stimulate a strong immune response against an individual patient's malignant cells. Each therapy is tumor-specific so, in theory, cells other than tumor cells are not be affected. There are several kinds of patient-specific vaccines under investigation that use antigens from a patient's own tumor cells.

Prostate Specific Antigen (PSA) is a prostate-specific protein antigen that can be found circulating in the blood, as well as on prostate cancer cells. PSA generally is present in small amounts in men who do not have cancer, but the quantity of PSA generally rises when prostate cancer develops. The higher a man's PSA level, the more likely it is that cancer is present, but there are many other possible reasons for an elevated PSA level. Patients have been shown to mount T-cell responses to PSA.

Sialyl Tn (STn) is a small, synthetic carbohydrate that mimics the mucin molecules (the primary molecule present in mucus) found on certain cancer cells.

Heat Shock Proteins (HSPs) (e.g., gp96) are produced in cells in response to heat, low sugar levels and other stress signals. In addition to protecting against stress, these molecules are also involved in the proper processing, folding, and assembling of proteins within cells. In laboratory experiments, HSPs from mouse tumors, in combination with small peptides, protected mice from developing cancer. The human vaccine consists of heat shock protein and associated peptide complexes isolated from a patient's tumor. HSPs are under investigation for treatment of several cancers including liver, skin, colon, lung, lymphoma and prostate cancers.

Ganglioside molecules (e.g., GM2, GD2, and GD3) are complex molecules containing carbohydrates and fats. When ganglioside molecules are incorporated into the outside membrane of a cell, they make the cell more easily recognized by antibodies. GM2 is a molecule expressed on the cell surface of a number of human cancers. GD2 and GD3 contain carbohydrate antigens expressed by human cancer cells.

Carcinoembryonic antigen (CEA) is found in high levels on tumors in people with colorectal, lung, breast and pancreatic cancer as compared with normal tissue. CEA is thought to be released into the bloodstream by tumors. Patients have been shown to mount T-cell responses to CEA.

MART-1 (also known as Melan-A) is an antigen expressed by melanocytes—cells that produce melanin, the molecule responsible for the coloring in skin and hair. It is a specific melanoma cancer marker that is recognized by T cells and is more abundant on melanoma cells than normal cells.

Tyrosinase is a key enzyme involved in the initial stages of melanin production. Studies have shown that tyrosinase is a specific marker for melanoma and is more abundant on melanoma cells than normal cells.

Prevention Vaccines

Viral proteins on the outside coat of cancer-causing viruses are commonly used as antigens to stimulate the immune system to prevent infections with the viruses.

Adjuvants

To heighten the immune response to cancer antigens, researchers usually attach a decoy substance, or adjuvant, that the body recognizes as foreign. Adjuvants are weakened proteins or bacteria which “trick” the immune system into mounting an attack on both the decoy and the tumor cells. Several adjuvants are described below:

Keyhole limpet hemocyanin (KLH) is a protein made by a shelled sea creature found along the coast of California and Mexico known as a keyhole limpet. KLH is a large protein that both causes an immune response and acts as a carrier for cancer cell antigens. Cancer antigens often are relatively small proteins that can be invisible to the immune system. KLH provides additional recognition sites for immune cells known as T-helper-cells and can increase activation of other immune cells known as cytotoxic T-lymphocytes (CTLs).

Bacillus Calmette Guerin (BCG) is a live-attenuated form of M. bovis, a Mycobacterium species closely related to the tuberculosis bacterium. BCG is added to some cancer vaccines to boost the immune response to the vaccine antigen. BCG is especially effective for eliciting immune response, which can involve the ability of BCG to recruit and activate natural killer (NK) cells, polymorphonuclear leukocytes (PMNs), and other cells of the innate immune response. BCG has been used for decades as a vaccine against tuberculosis.

Interleukin-2 (IL-2) is a protein made by the body's immune system that can boost the cancer-killing abilities of certain specialized immune system cells called natural killer cells. Although it can activate the immune system, many researchers believe IL-2 alone is not enough to prevent cancer relapse. Several cancer vaccines use IL-2 to boost immune response to specific cancer antigens.

Granulocyte Monocyte-Colony Stimulating Factor (GM-CSF) is a protein that stimulates the proliferation of antigen-presenting cells.

QS21 is a plant extract that, when added to some vaccines, can improve the body's immune response.

Montanide ISA-51 is an oil-based liquid intended to boost an immune response.

In addition to the FDA-approved Hepatitis B vaccine and HPV vaccine, there are other vaccines currently under investigation that have the potential to reduce the risk of cancer. These vaccines target infectious agents that cause cancer, similar to traditional prophylactic vaccines that target other disease-causing infectious agents, such as those that cause polio or measles. Non-infectious components of cancer-causing viruses, commonly the viral coat proteins (proteins on the outside of the virus), serve as antigens for these vaccines. These antigens can stimulate the immune system in the future to attack cancer-causing viruses, which are, in turn, reduce the risk of the associated cancer.

The following is a summary of ongoing or unpublished Phase III trials. The information is derived from government databases including the National Cancer Institute's clinical trials database, www.cancer.gov/clinicaltrials/search, and the National Institutes of Health clinical trials Web site, www.clinicaltrials.gov. Information about each trial also can be obtained by clicking the links in the far right column of the table.

### Phase III Vaccine Trials

| Type of | Vaccine Name (if | | |
|---|---|---|---|
| Cancer | applicable) | Lead Institution | Nature of Vaccine |
| Cervical | Gardasil ™ HPV | Merck & Co. | The HPV quadrivalent vaccine contains viral |
| Cancer | (human papilloma | | proteins from four HPV types: HPV 16 & 18, |
| | virus) quadrivalent | | the types that account for about 70% of the |
| | vaccine | | worldwide cases of cervical cancer, and HPV |
| | | | 6 & 11, the types most commonly associated |
| | | | with genital warts. |
| Cervical | Cervarix ™ HPV | National Cancer | The HPV bivalent vaccine (provided to NCI |
| Cancer | bivalent vaccine | Institute (in | for this trial by GlaxoSmithKline Biologicals) |
| | | collaboration with | contains viral proteins from two HPV types: |
| | | Costa Rican | HPV 16 & 18, the types that account for |
| | | investigators) | about 70% of the worldwide cases of cervical |
| | | | cancer. |
| Follicular B- | Biovaxid ® | National Cancer | The vaccine is composed of antibodies that |
| cell Non- | | Institute | are unique to a patient's own tumor cells. |
| Hodgkin's | | | These idiotype proteins are chemically |
| Lymphoma | | | attached to the adjuvant protein keyhole |
| | | | limpet hemocyanin (KLH). GM-CSF |
| | | | (granulocyte macrophage colony stimulating |
| | | | factor) is used to enhance the immune |
| | | | response against the idiotype proteins. |
| Follicular B- | GTOP-99 MyVax ® | Genitope | The vaccine consists of antibodies that are |
| cell Non- | Personalized | Corporation | unique to a patient's tumor. These idiotype |
| Hodgkin's | Immunotherapy | | proteins are chemically attached to the |
| Lymphoma | | | adjuvant protein KLH. Sargramostim (GM- |
| | | | CSF) is also used to enhance the immune |
| | | | response. |
| Kidney | Oncophage ™ | Antigenics, Inc. | The vaccine -- heat shock protein (gp96) and |
| Cancer | (HSPPC-96) | | associated peptides -- is made from each |
| | | | patient's own tumor. |
| Cutaneous | Oncophage ™ | Antigenics, Inc. | The vaccine -- heat shock protein (gp96) and |
| Melanoma | (HSPPC-96) | | associated peptides -- is made from each |
| | | | patient's own tumor. |
| Cutaneous | Not Named | European | The vaccine consists of GM2, a common |
| Melanoma | | Cooperative | antigen on melanoma cells, which is |
| | | (EORTC) | conjugated to the adjuvant KLH. QS21 is |
| | | | used to enhance the immune response. |
| Cutaneous | Not Named | National Cancer | The vaccine contains a combination of three |
| Melanoma | | Institute | melanocyte-specific antigens: tyrosinase, |
| | | | gp100, and MART. Sargramostim (GM-CSF) |
| | | | is used to enhance the immune response. |
| Cutaneous | Not Named | National Cancer | The vaccine contains gp100, IL-2, and |
| Melanoma | | Institute | Montanide ISA-51. Montanide ISA-51 is an |
| | | | oil used to enhance the immune response. |
| Cutaneous | MDX-1379 | Medarex, Inc. | The vaccine contains gp100. MDX-010 is an |
| Melanoma | | | anti-cytotoxic T lymphocyte antigen-4 |
| | | | (CTLA-4) monoclonal antibody, also known |
| | | | as ipilumumab. CTLA-4 helps suppress |
| | | | immune responses; blocking its activity with |
| | | | MDX-010 can improve the immune response |
| | | | induced by MDX-1379. |
| Ocular | Not Named | European | The vaccine contains several melanoma |
| Melanoma | | Cooperative | differentiation peptides. |
| | | (EORTC) | |
| Prostate | GVAX ® | Cell Genesys, Inc. | Cells from two, patient-non-specific prostate |
| Cancer | | | cancer cell lines that have been genetically |
| | | | engineered to overexpress and secrete GM- |
| | | | CSF, which stimulates the immune response |
| | | | to vaccines. |
| Prostate | GVAX ® | Cell Genesys, Inc. | Cells from two, patient-non-specific prostate |
| Cancer | | | cancer cell lines that have been genetically |
| | | | engineered to overexpress and secrete GM- |
| | | | CSF, which stimulates the immune responses |
| | | | to vaccines. |
| Prostate | Provenge ® | National Cancer | A patient's own immune system cells trained |
| Cancer | sipuleucel T | Institute | in the laboratory to target the protein prostatic |
| | | | acid phosphatase (PAP), which is made by |
| | | | prostate cells |
| Multiple | Not Named | University of | Fragments from two tumor proteins called |
| Myeloma | | Arkansas | MAGE-A3 and NY-ESO-1, which are found |
| | | | in myelomas and other tumors and which |
| | | | have been shown to stimulate antitumor |
| | | | immune responses. |

Immunotherapy of cancer patients with Bacillus Calmette-Guérin has been conducted in other countries (Immunotherapy of cancer patients with Bacillus Calmette-Guérin: summary of four years of experience in Japan. Torisu et al. Ann NY Acad Sci. 1976; 277(00):160-86). In this study, active immunotherapy with living BCG was conducted on 98 patients with various types of cancer. The candidates for this therapy were patients with residual or inoperable cancer of the colorectum, liver, breast, biliary tract, lung, and other organs with a follow-up of 4-58 months. Eleven of the 98 (11%) were able to survive for as long as 37-58 months (mean survival time 42.5 months) because of this treatment and as of the writing of this paper were still living. Another 11 patients were also alive more than 24 months after starting treatment. Thirty-seven patients, however, succumbed within 12 months despite BCG immunotherapy. On the other hand, 37 patients in the control group, who shared the same clinical status and did not receive BCG therapy during this period, succumbed 2-12 months (mean survival time 8.7 months). The pretreatment immunoresponsiveness of these 98 patients was suppressed, as measured by the following immunologic parameters: T-cell subpopulation in the peripheral blood, stimulation index of PHA, and skin tests to DNCB, KLH, PPD, and PHA. All of these parameters improved shortly after initiation of BCG injections in 22 patients who survived more than 24 months. In contrast, in patients who died within 12 months, immunoresponsiveness remained suppressed throughout the course. Thus, there was an apparent correlation between the effectiveness of BCG and immunoresponsiveness, a finding that indicates a more immunogenic paBCG vaccine can be even more effective when administered to patients with advanced, inoperable cancer. In addition, a good correlation was observed between the duration of inflammatory reactions at BCG injection sites and clinical prognoses. Moreover, it was shown that a relatively high amount of BCG (20-80 mg as an initial dosage) and repeated injections of living BCG were necessary to obtain a sufficient enhancing effect on the immunocompetency of these late-stage cancer patients. The most conventional criterion used to determine an optimal time for booster injections of BCG was measurement of the PPD-evoked skin reaction at the BCG injection site, that is, evidence of delayed-type hypersensitivity to tuberculin. When a marked flare-up reaction of more than 2.5×2.5 cm in size was observed, the effect of BCG was considered to be continuing, and no additional booster injection was needed. The mean interval between the first and second BCG injections was 6.2+1−1.1 months in patients who survived more than 2 years. In contrast, the &ration of this reaction was only transient in ineffective cases. The most frequent side effects of this therapy were fever and malaise; these complications occurred in 62% of the cases. No severe side effects, such as dissemination, anaphylactic shock, or granulomatous hepatitis, were reported in this study, even in patients to whom a total dosage of more than 200 mg of living BCG were injected.

It are be recognized that the methods (e.g., the modes of administration, dosages and time courses of administration) described above using BCG and those described in the examples are applicable in the treatment of cancer using paBCG. Additional modes of administration and dosages are described herein and applicable to the present methods.

Parenteral administration of the compositions of the present invention, if used, is generally characterized by injection. Injectables can be prepared in conventional forms, either as liquid solutions or suspensions, solid forms suitable for solution of suspension in liquid prior to injection, or as emulsions. As used herein, “parenteral administration” includes intradermal, subcutaneous, intramuscular, intraperitoneal, intravenous, intra-articular and intratracheal routes.

The dosage of the composition varies depending on the weight, age, sex, and method of administration. In one embodiment, the dosage of the compound is from 0.5×10² colony-forming units to 5×10⁸ colony-forming units of the viable live-attenuated microbial strain. More preferably, the compound is administered in vivo in an amount of about 1×10⁶ colony-forming units to 5×10⁷ colony-forming units of the viable live-attenuated microbial strain. The dosage can also be adjusted by the individual physician as called for based on the particular circumstances.

The compositions can be administered conventionally as vaccines containing the active composition as a predetermined quantity of active material calculated to produce the desired therapeutic or immunologic effect in association with the required pharmaceutically acceptable carrier or diluent e., carrier or vehicle). By “pharmaceutically acceptable” is meant a material that is not biologically or otherwise undesirable, i. e., the material can be administered to an individual along with the selected composition without causing any undesirable biological effects or interacting in a deleterious manner with any of the other components of the pharmaceutical composition in which it is contained.

Although the examples provided below involve modifications of BCG, the current vaccine against tuberculosis, the invention teaches how vaccines of other intracellular pathogens can be developed by expressing dominant-negative mutants of anti-apoptotic bacterial enzymes.

### Expression of Dominant-Negative Mutants of Microbial Anti-Apoptotic Enzymes

The primary utility of a dominant-negative approach over allelic inactivation for reducing the activity of an anti-apoptotic microbial enzyme is when the gene appears to be essential for survival of the microbe in vitro despite attempts to enrich the media in which the microorganism is cultivated. In these circumstances, allelic inactivation interferes with cultivation of the mutant bacterium and make it unsuitable as a vaccine strain, and a method for rendering a partial phenotype with reduced activity of the essential enzyme that still enables the microbe to grow is favored. Antisense techniques and targeted incremental attenuation have been previously described in WO 02/062298 and can be used to reduce the activity of an essential microbial enzyme. The expression of dominant-negative enzyme mutants represents an alternative strategy that shares many of the methods described for practicing targeted incremental attenuation but differs in some important aspects.

### Step 1. Identification of Anti-Apoptotic Microbial Enzymes

Detailed methods for identifying essential and anti-apoptotic microbial enzymes have been described in WO 02/062298. To verify that reducing the activity of the microbial enzyme renders a pro-apoptotic effect, host cell apoptosis can be monitored using either in vitro cell culture techniques (e.g., infected macrophages) or the recovery of cells or tissue of infected animals in vivo. There are a large number of techniques used to monitor apoptosis including flow cytometry, TUNEL stains, and DNA fragmentation assays that are well-known to those skilled in the art.

There are two important differences related to the selection of anti-apoptotic enzymes for practicing a dominant-negative strategy as compared to targeted incremental attenuation.

First, for the dominant-negative approach it is best to select enzymes with known multimeric structure, whereas this is not important for practicing targeted incremental attenuation. This is because in the former the mechanism of reduced enzyme activity is believed to be mediated by interference by mutant enzyme monomers with either the formation of the enzymatically-active multimer or an alteration in tertiary configuration that adversely affects enzyme activity. A body of published literature demonstrates that several bacterial enzymes that inactivate host-derived oxidants and thus are likely to have anti-apoptotic effects are multimers in their biologically active form including, but not limited to the iron co-factored superoxide dismutase of M. tuberculosis/bovis/BCG, thioredoxin, glutamine synthase, and bacterial glutaredoxin (glutathione reductase).

Thus, with each of these enzymes one can reduce enzymatic activity by using a dominant-negative approach as taught in the current invention. Reducing SodA activity by using anti-sense techniques as described in results in stronger host immune responses and greater vaccine-induced protection against infection. Reducing SodA activity by a dominant-negative strategy has a similar effect.

Second, although practice of the dominant-negative strategy and targeted incremental attenuation are not limited to essential microbial genes, that is the primary reason for preferring targeted incremental attenuation over simple allelic inactivation when the gene is essential. In contrast, there are some potential advantages of employing a dominant-negative strategy over allelic inactivation in some microorganisms even for non-essential genes. First, there are considerations of time and the ease of genetic modifications that are especially true for species in which it is difficult to achieve homologous recombination necessary for allelic inactivation, but for which overexpression of a gene can be accomplished on plasmids or other vectors. Another reason for selecting overexpression of a dominant-negative enzyme mutant over allelic inactivation is if the enzyme is an important immunogen. In this situation, it is important to allow the vaccine strain to continue to produce the enzyme as it can be a target against which an immune response can be directed. Thus, when the host subsequently becomes infected with the pathogen causing a disease that the vaccine is intended to prevent, the host has a more complete repertoire of immune responses to direct against the pathogen. This “antigen repertoire” consideration is unimportant under circumstances when the pro-apoptotic live-attenuated vaccine strain is used solely as a vector for expressing exogenous antigens, and the desired immune response is against the exogenous antigen.

Among the mycobacterial enzymes with known or suspected anti-apoptotic effects listed above, SodA and GlnA1 (glutamine synthase) appear to absolutely essential for bacterial growth. Thus, they are not good candidates for allelic inactivation for the purpose of making a vaccine but can be manipulated to achieve a partial reduction in enzyme activity achieved either through antisense techniques, targeted incremental attenuation, or a dominant-negative approach. As both SodA and GlnA1 have been implicated in immune evasion by M. tuberculosis and are also produced by BCG, they are favored targets for enhancing the immunogenicity of BCG. Examples below show that the SodA-diminished phenotype in BCG is also associated with enhanced vaccine efficacy.

### Step 2. Generating Mutants of Anti-Apoptotic Enzymes

The methods for generating mutants of anti-apoptotic enzymes for practicing the dominant-negative strategy include those described in WO 02/062298 but also involve an important difference. In the targeted incremental attenuation strategy, the mutant enzyme is the sole source of enzyme activity. These mutants can exhibit enzymatic activity that is only, for example, 95%, 90%, 85%, 80%, 75%, 70%, 65%, 60%, 55%, 50%, 45%, 40%, 35%, 30%, 25%, 20%, 15%, 10%, 5%, 2%, etc. of the activity of the parent, natural enzyme. A series of mutant enzymes can be produced that have activities that fall within this range of reduction in activity. Thus, for essential enzymes where the practice of targeted incremental attenuation has its greatest utility, the mutant enzyme has some activity.

In contrast, in the dominant-negative strategy, the mutant enzyme can be completely inert, exhibiting 0% activity. This is because the dominant-negative strategy is based on interference between expressed mutant enzyme monomers and the wild-type enzyme monomers encoded by the parent gene. This interference leads to a reduction in total enzyme activity.

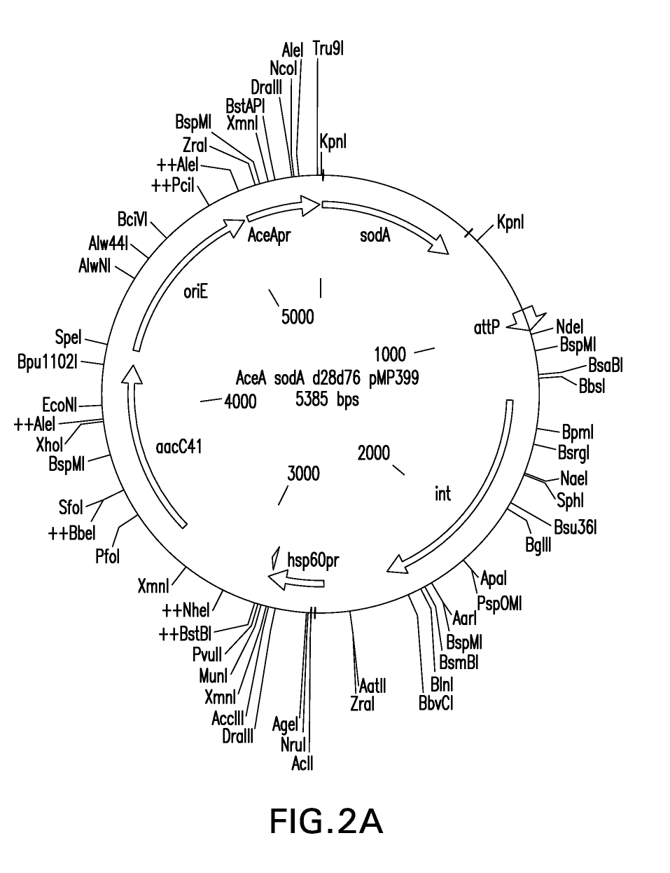

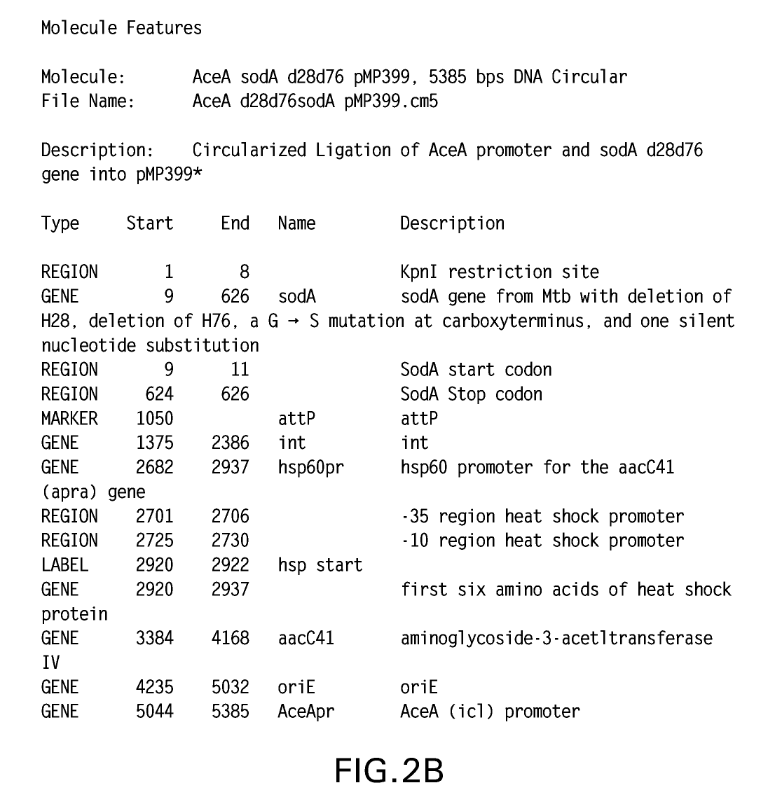

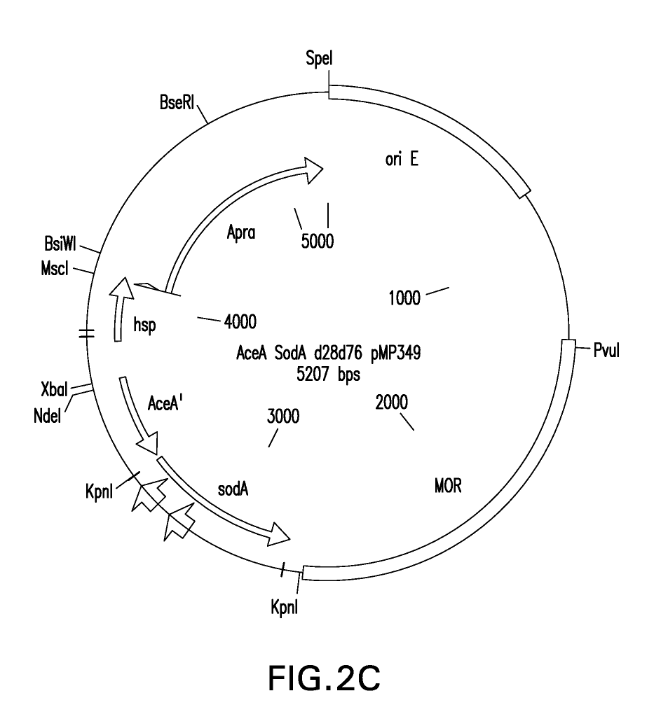

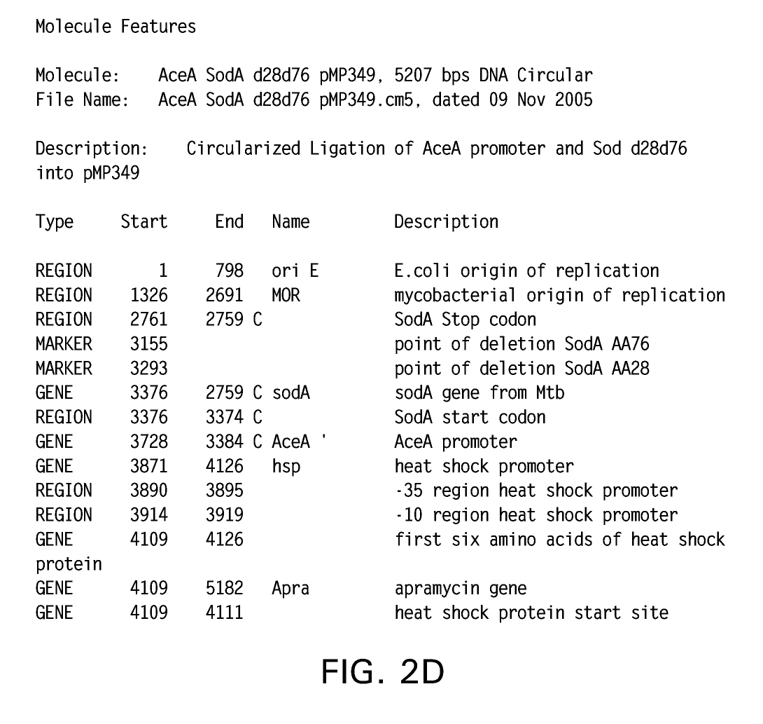

This difference has implications for the design of enzyme mutants to practice the dominant-negative strategy versus targeted incremental attenuation. Most notably, mutant enzymes used in the dominant-negative strategy are potentially easier to design as one strategy is simply to disable the active site of the enzyme. As noted in WO 02/062298, Xray crystallographic data are available for many of the bacterial enzymes that inactivate host oxidants, including identification of active site residues. Thus, information is available to help guide the construction of enzyme mutants in which active site residues are eliminated or replaced. This strategy was employed in the construction of a ΔH28ΔH76 mutant of SodA, in which two of the histidines that chelate the active site iron of SodA have been removed (FIG. 2, Example 1).

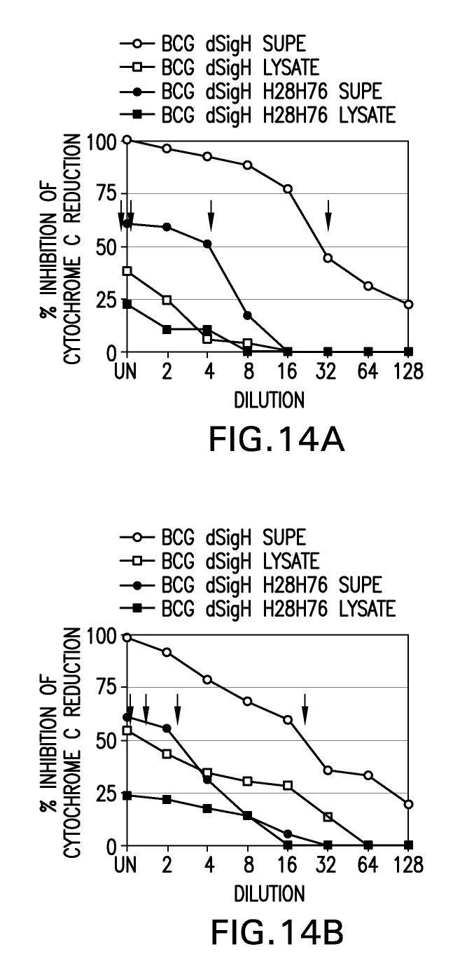

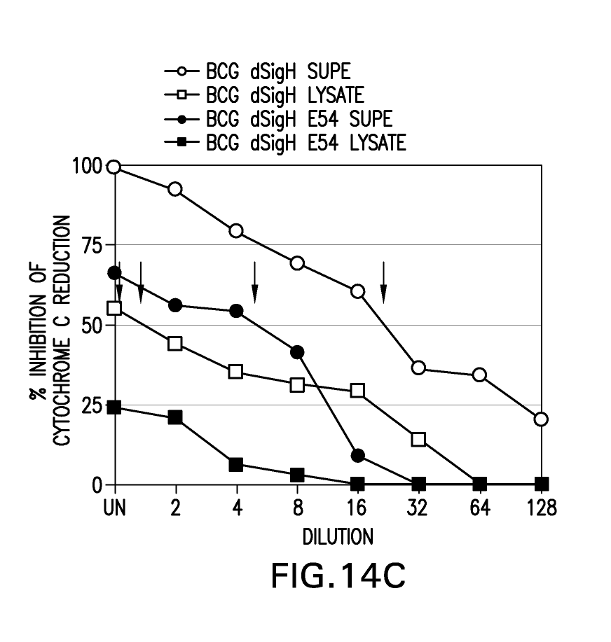

Also, in multimeric enzymes, for example glutamine synthase which has a dodecameric structure, the active site frequently lies between monomers and is formed by components of more than one monomer. This enables mutant enzymes to be designed in which the monomer has amino acid deletions, insertions, or substitutions that affect more than one active site. This strategy was employed in the construction of a ΔD54ΔE335 mutant of glnA1, which encodes the primary glutamine synthase of M. tuberculosis and BCG (FIG. 14).

However, some of the mutant enzymes constructed to practice targeted incremental attenuation can also be used to practice the dominant-negative strategy. For example, sodA mutant alleles on pLou1-mut-SodA (Table 1) were being placed into BCG to construct BCG(pLou1-mut SodA) (Table1) using techniques for targeted incremental attenuated described in WO 02/062298 when the recombinant BCG strains were noted to have reduced SOD activity (Example 1).

The genes encoding mutant enzymes with reduced enzymatic activity can have single or multiple nucleotide differences compared to the wild-type gene leading to single or multiple amino acid deletions, insertions, and/or substitutions. Nucleotide differences can be introduced using the wild-type gene as a substrate and using a variety of techniques to achieve site-directed mutagenesis known to those skilled in the art including PCR-based methods. Alternatively, the gene containing desired mutations can be synthesized de novo.

When the invention is used in persons previously infected with a live Mycobacterium, or alternatively to prepare the subject for subsequent administration of live Mycobacterium as immunotherapy for cancer, it is not necessary to proceed to Step 3. In this circumstance the anti-apoptotic enzyme or a mutant of the enzyme can be administered directly to the subject for the purpose of inducing antibodies or cellular immune responses that interfere with the activity of the anti-apoptotic enzyme produced by the live bacteria in vivo.

### Step 3: Expression of the Mutant Enzyme by the Microbe

When the invention involves the use of stably-modified live-attenuated bacteria with enhanced immunogenicity directly as immunotherapy against cancer, or as an adjuvant to be mixed with another vaccine, Steps 3 and 4 are also practiced. Next, the gene encoding the mutant enzyme is incorporated into a vector that either integrates into the chromosome of the bacterium or can be stably maintained as a plasmid within the bacterium. Methods for expressing DNA in BCG and other mycobacteria have been available since 1987, are well-known to those skilled in the art, and include techniques taught by Bloom et al (U.S. Pat. No. 5,504,005, Recombinant mycobacterial vaccine; U.S. Pat. No. 5,854,055 and U.S. Pat. No. 6,372,478, Recombinant mycobacteria), which are hereby incorporated by reference in their entirety for their teaching regarding methods for expressing DNA).

Step 4: Identifying Mutant Bacteria to Use as a Vaccine, as a Host Strain to Express a Heterologous Antigen, as Cancer Immunotherapy, or as Treatment for Fibrosing Lung Disease Induced by Infection with a Mycobacterium Species

Methods for identifying mutant bacteria to use as a vaccine are described in detail in WO 02/062298 and primarily involve observing a response in an animal model that correlates with enhanced vaccine-induced protection, for example, enhanced immune responses.

Another method for evaluating mutant bacterial strains for their function as a vaccine strain or as a vector for delivering exogenous antigens involves assays to determine the degree of reduction in enzyme activity in vitro. Reduction in the activity of an enzyme that normally renders an anti-apoptotic effect upon the host results in increased host cell apoptosis when that bacterium is used to vaccinate a host animal, and is a more immunogenic vaccine than the parent bacterium. Thus, measuring enzyme activity in lysates and/or supernatants of parent bacterium and the mutant bacterium can be used to indicate whether dominant-negative expression of a specific mutant enzyme has produced the desired reduction in total enzyme activity. Total enzyme activity is reduced by the dominant-negative strategy and prior observations link enhanced vaccine efficacy to reduced enzyme activity achieved by another technique, for example antisense techniques, thus the bacterium with the dominant-negative enzyme reduction is a more efficacious vaccine strain.

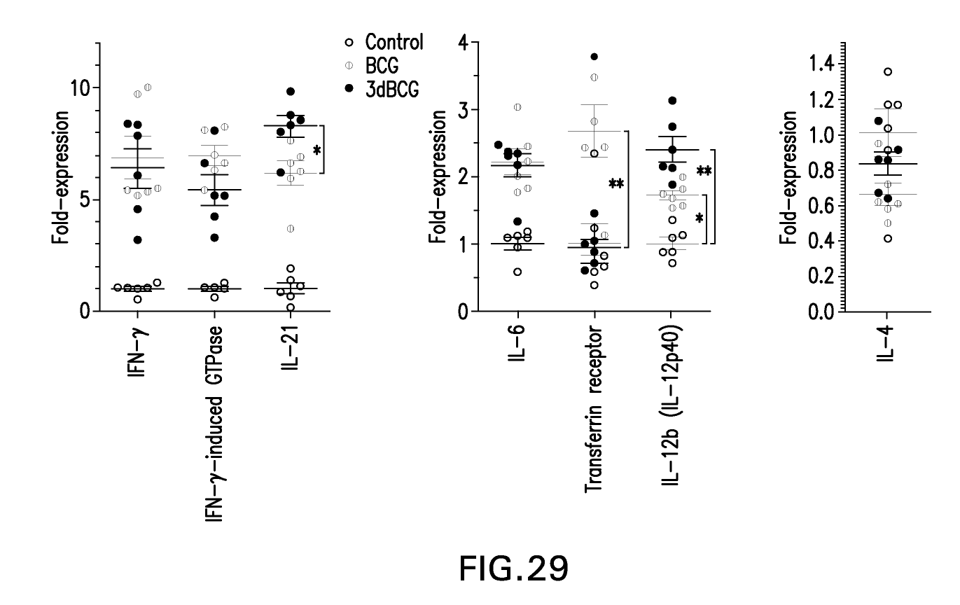

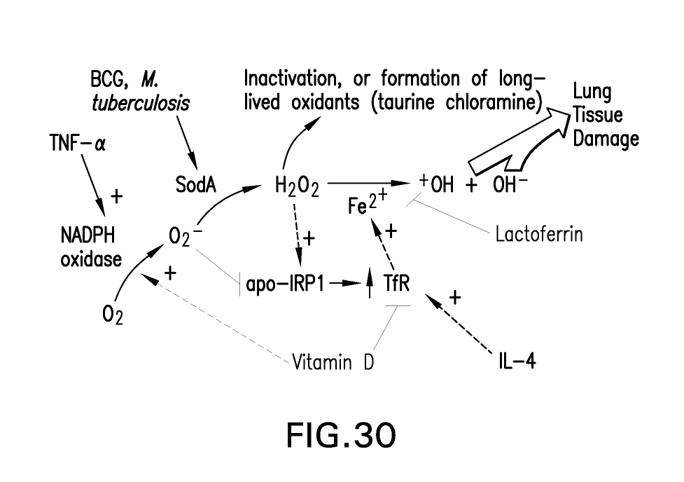

As noted above, vaccines in which a dominant-negative enzyme mutant is over-expressed are preferable to allelic inactivation if the enzyme is an important immunogen. In this situation, it is important to allow the vaccine strain to continue to produce the enzyme, albeit with diminished enzymatic activity, as it is a target against which an immune response can be directed. Given the effect of mycobacterial SodA upon the expression of transferrin receptors (FIG. 29) and the effect of iron uptake by macrophages in promoting tissue damage in the lung (FIG. 30), this consideration is particularly important when paBCG is to be used as a vaccine to prevent the conversion of latent tuberculosis into active pulmonary tuberculosis, or to prevent lung fibrosis as a complication of lung infection with other Mycobacterium species.

Elimination of Sigma Factors and Other Regulatory Genes that Govern the Production of Microbial Anti-Apoptotic Enzymes

### Step 1. Identification of Regulatory Genes of Anti-Apoptotic Microbial Enzymes

The production of some microbial anti-apoptotic enzymes is under the control of regulatory genes including sigma factors that govern the transcription of multiple genes via an effect upon promoter regions. Thus, allelic inactivation of such genes represents an additional way to reduce the production of anti-apoptotic microbial enzymes, with the potential for a pleiotropic effect in which the activity of several anti-apoptotic enzymes is reduced by a single genetic manipulation.

Regulatory genes can be identified by their effect upon the expression of other microbial factors, including anti-apoptotic enzymes. The screening of transposon and other random mutagenesis libraries for mutants that result in enhanced apoptosis of infected cells not only yields mutants with direct defects in anti-apoptotic enzymes but can also identify mutations in regulatory genes that influence the production of key anti-apoptotic microbial enzymes. There is strong homology amongst regulatory factors from different species and some investigators have identified novel sigma factors based on homology to known sigma factors by DNA or amino acid sequence.

The allelic inactivation of the gene encoding sigma factor H (sigH) of M. tuberculosis has been described [Kaushal, D. et al, 2002; Manganelli, R. et al, 2002; Raman, S. et al, 2001, incorporated herein by reference for their teaching of methods to inactivate sigH]. Inactivation of sigH was accompanied by an effect upon several mycobacterial enzymes including thioredoxin, thioredoxin reductase, and a glutaredoxin homolog. A sigH deletion was introduced into the chromosome of BCG, as described below. The enhanced efficacy of BCGΔsigH as a vaccine is described below.

Another modification that enhances BCG vaccine efficacy is the inactivation of sigE. This can be done alone or in addition to sigH inactivation. sigE inactivation also plays a role in the resistance of M. tuberculosis to oxidative stress and methods for inactivating sigE have been described in M. tuberculosis [Manganelli, R. et al, 2001; Manganelli, R. et al, 2004b; Manganelli, R. et al, 2004a, incorporated herein by reference for their teaching of methods to inactivate sigE].

### Step 2. Inactivation of Regulatory Genes of Anti-Apoptotic Microbial Enzymes

The inactivation of regulatory and sigma factor genes can be performed using allelic inactivation techniques involving suicide plasmid vectors or mycobacteriophage-derived genetic tools that are capable of replicating as a plasmid in E. coli and lysogenizing a mycobacterial host. These methods and tools are well-known to those skilled in the art.

Specific methods for inactivating sigH and sigE in M. tuberculosis have already been described by several groups of investigators as noted above. The methods employed herein in allelic inactivation of sigH in BCG are shown below.

These examples show the enhancement of immunogenicity of bacteria by inactivating regulatory genes, which results in the reduced activity of anti-apoptotic microbial enzymes.

### Examples of Pro-Apoptotic BCG Vaccines

Examples of the microbes made by overexpression of mutant SOD include, but are not limited to the following: a mutant M. tuberculosis or BCG in which glutamic acid is deleted at position 54 of superoxide dismutase; a mutant M. tuberculosis or BCG in which glutamic acid is deleted at position 54 and histidine at position 28 is replaced by arginine of superoxide dismutase; a mutant M. tuberculosis or BCG in which histidine is deleted at position 28 of superoxide dismutase; a mutant M. tuberculosis or BCG in which histidine is deleted at position 76 of superoxide dismutase; a mutant M. tuberculosis or BCG is which histidines are deleted at position 28 and at position 76 of superoxide dismutase, a mutant M. tuberculosis or BCG in which histidines are deleted at position 28 and at position 76 of superoxide dismutase and there is a glycine to serine substitution at the carboxyterminus.

Examples of the microbes made by overexpression of glutamine synthetase (glnA1) include, but are not limited to the following: a mutant M. tuberculosis or BCG in which aspartic acid is deleted at position 54 of glutamine synthase; a mutant M. tuberculosis or BCG in which glutamic acid is deleted at position 335 of glutamine synthase; a mutant M. tuberculosis or BCG in which aspartic acid is deleted at position 54 and glutamic acid is deleted at position 335 of glutamine synthase.

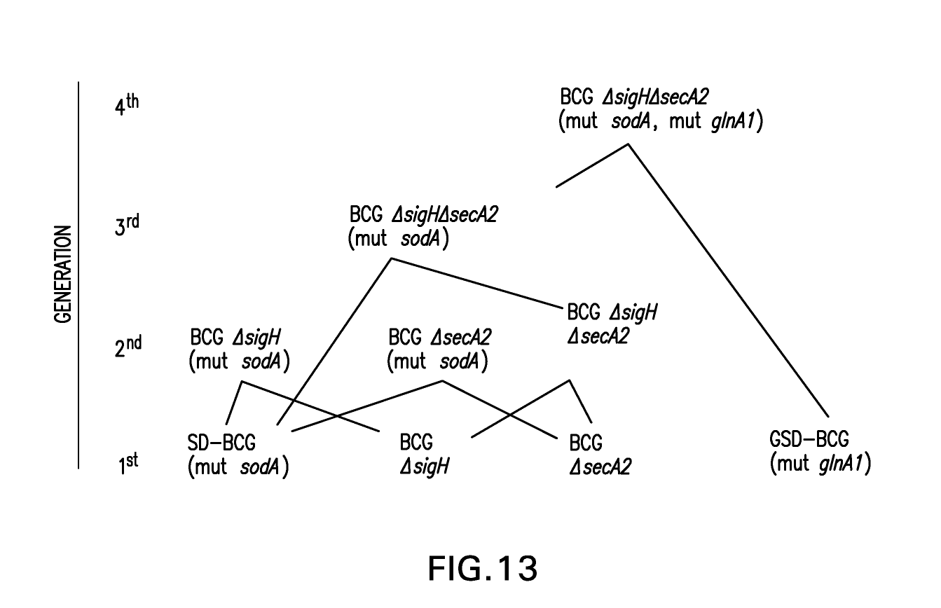

The microbes of the disclosed methods and compositions can be constructed using the disclosed generational approach to bacterial modification (FIG. 13). The list below shows additional combinations of the preferred modifications for introducing into BCG the pro-apoptotic phenotype associated with enhanced immunogenicity.

### 1^{st} Generation:

a. SAD-BCG (also referred to as: “SD-BCG [mut sodA]”)
b. SIG-BCG (also referred to as: “BCGΔsigH”)
c. SEC-BCG (also referred to as: “BCGΔsecA2”)
d. GLAD-BCG (also referred to as: “GSD-BCG [mut glnA1])

### 2^{nd} Generation:

a. SAD-SIG-BCG (also referred to as: “BCGΔsigH [mut sodA]”)
b. SAD-SEC-BCG (also referred to as: “BCGΔsecA2 [mut sodA]”)
c. DD-BCG (also referred to as: “BCGΔsigHΔsecA2”, “double-deletion BCG”)
d. GLAD-SIG-BCG (also referred to as: “BCGΔsigH [mut glnA1]”)
e. GLAD-SEC-BCG (also referred to as: “BCGΔsecA2 [mut glnA1]”)
f. GLAD-SAD-BCG (also referred to as: “BCG [mut sodA, mut glnA1])

### 3^{rd} Generation:

a. 3D-BCG (also referred to as: “BCGΔsigHΔsecA2 [mut sodA]”, “3^{rd}-generation BCG”). There are multiple contemplated 3D-BCG strains based on the nature of the dominant-negative mutant SodA that is expressed to reduce total SOD activity. The dominant-negative mutant sodA gene can be inserted into the chromosome of DD-BCG or expressed on a plasmid.
b. GLAD-DD-BCG (also referred to as: “BCGΔsigHΔsecA2 [mut glnA1]”)
c. GLAD-SAD-SIG-BCG (also referred to as: “BCGΔsigH [mut sodA, mut glnA1]”)
d. GLAD-SAD-SEC-BCG (also referred to as: “BCGΔsecA2 [mut sodA, mut glnA1]”)

### 4^{th} Generation:

4D-BCG (also referred to as: “BCGΔsigHΔsecA2 [mut sodA, mut glnA1]”, “4^{th}-generation BCG”. There are 4 major types of 4D-BCG. All involve the addition of dominant-negative sodA and glnA1 mutants to DD-BCG, but vary in where the genes are inserted.
   Form 1—the mutant sodA and glnA1 alleles are inserted into the chromosome
   Form 2—the mutant sodA and glnA1 alleles are expressed on a plasmid
   Form 3—the mutant sodA allele is inserted into the chromosome and the mutant glnA1 allele is expressed on a plasmid
   Form 4—the mutant sodA allele is expressed on a plasmid and the mutant glnA1 allele is inserted into the chromosome

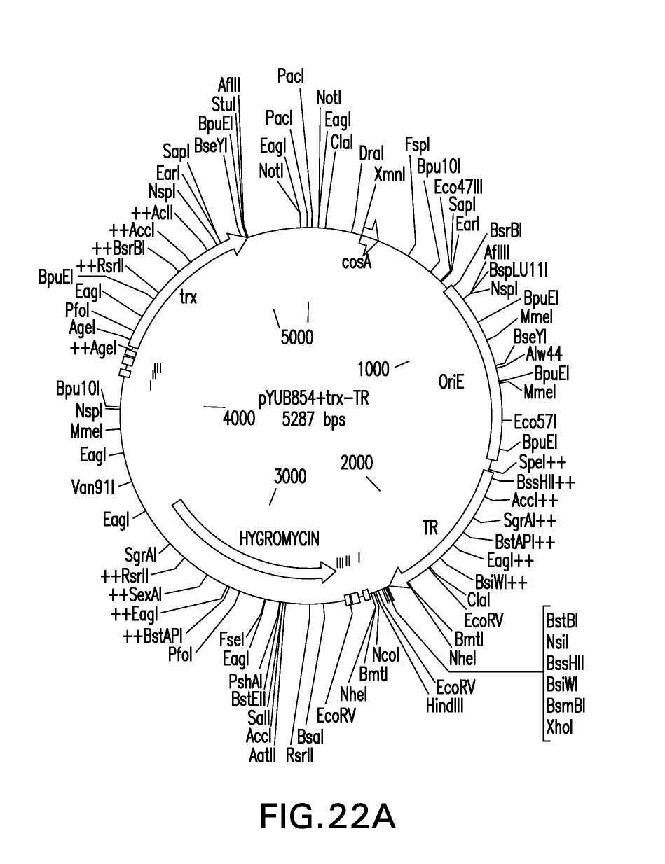

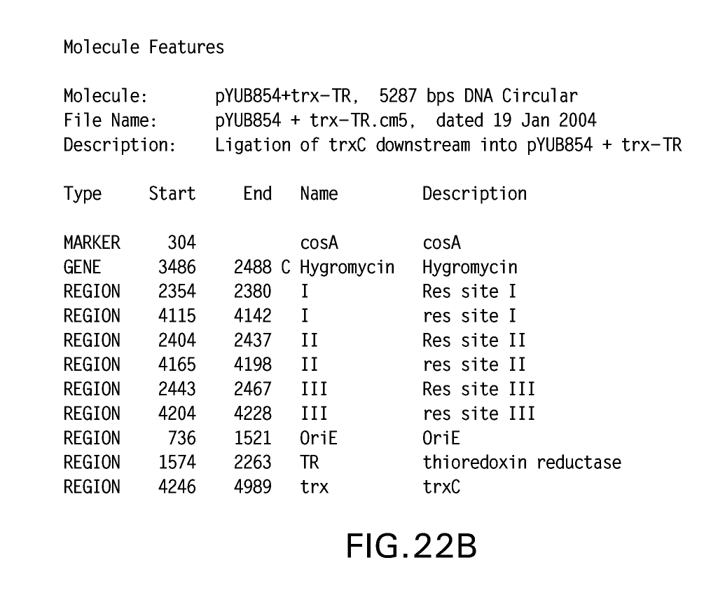

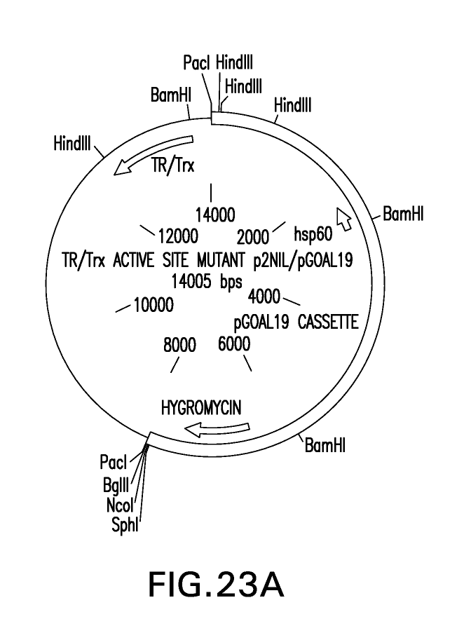

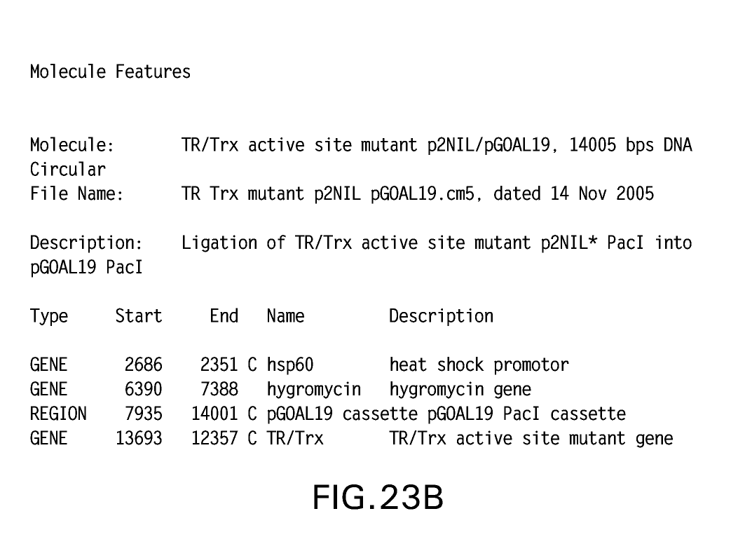

As inactivation of sigH affects the expression of multiple bacterial factors, some of which are important targets of the immune response, there are advantages to substituting the inactivation of sigH with the inactivation (or dominant-negative mutant enzyme expression) of one or more of the antioxidants whose expression is controlled by sigH. These include thioredoxin, thioredoxin reductase, a glutaredoxin homolog, and biosynthetic enzymes involved in the production of mycothiol, a small molecular weight reducing agent similar to mammalian gluthathione. This manipulation can have advantages over inactivating sigH when the pro-apoptotic BCG strain is used to vaccinate a host against tuberculosis, as the benefit of having the host respond to the sigH-controlled factors as immune targets may outweigh the benefit of having a vaccine strain that is less able to inhibit apoptosis. In contrast, the sigH-inactivated vaccines described herein are ideal for inducing strong innate responses that attract immune cells to the site of a cancer, for improving the immunogenicity of BCG used as an adjuvant, and as vectors to express exogenous antigens, as the presence of a complete or near-complete antigen repertoire of BCG is not important when the modified BCG strain is used primarily to induce an immune response against an exogenous antigen, e. g, for immunizing against other infectious agents or cancer antigens. To further teach how to practice the substitution of inactivating sigH-regulated anti-apoptotic genes instead of inactivating sigH, mutant alleles designed to inactivate thioredoxin and thioredoxin reductase are shown in FIG. 22 and FIG. 23. This approach is applicable to M. bovis strains other than BCG.

The paBCG vaccines disclosed herein are more immunogenic than the parent BCG vaccine strain. Furthermore, each vaccine generation exhibits progressive increases in immunogenicity. Compared to BCG they exhibit the following traits:

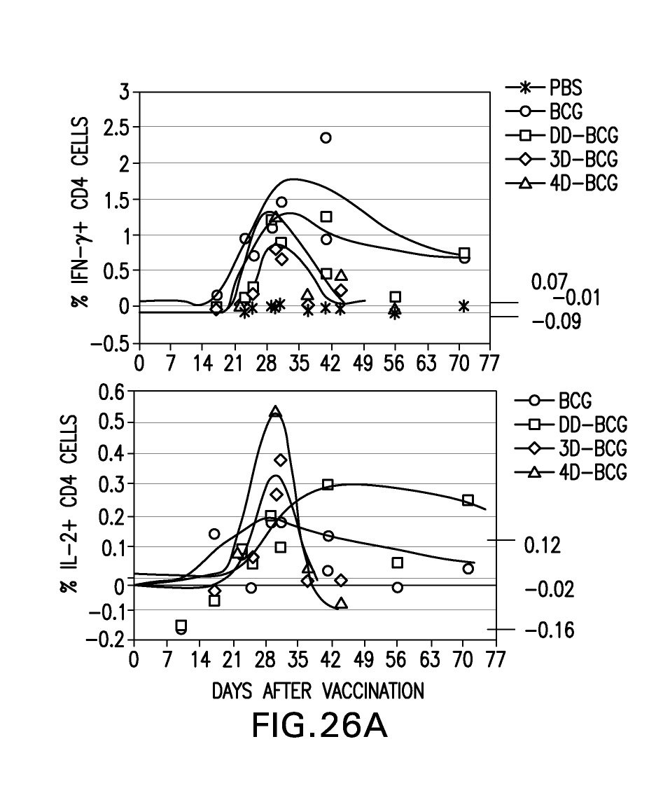

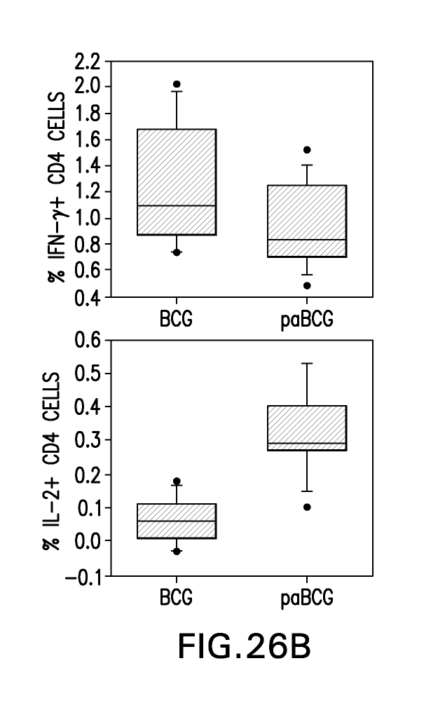

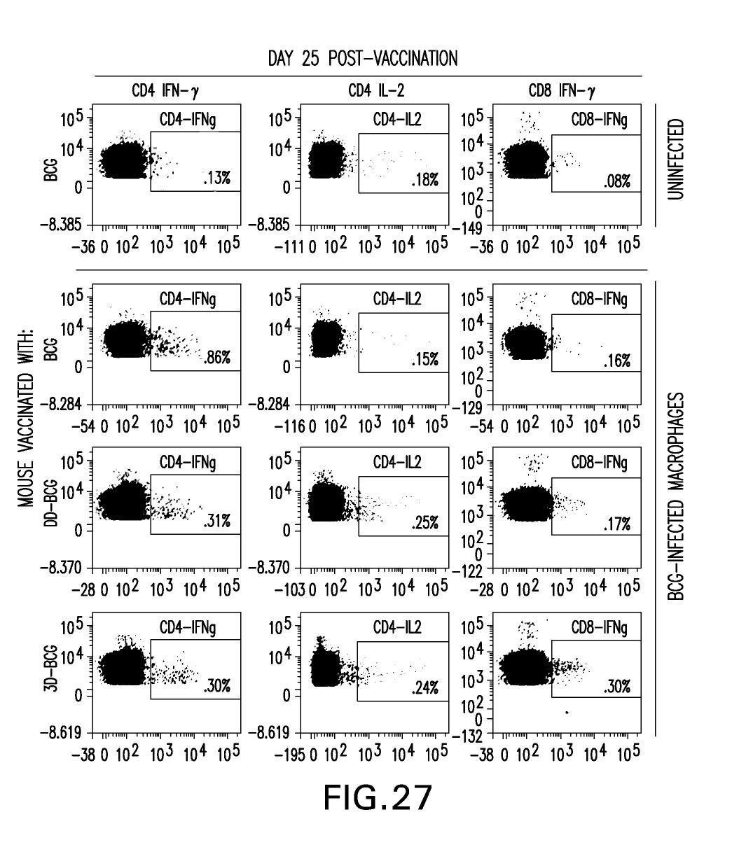

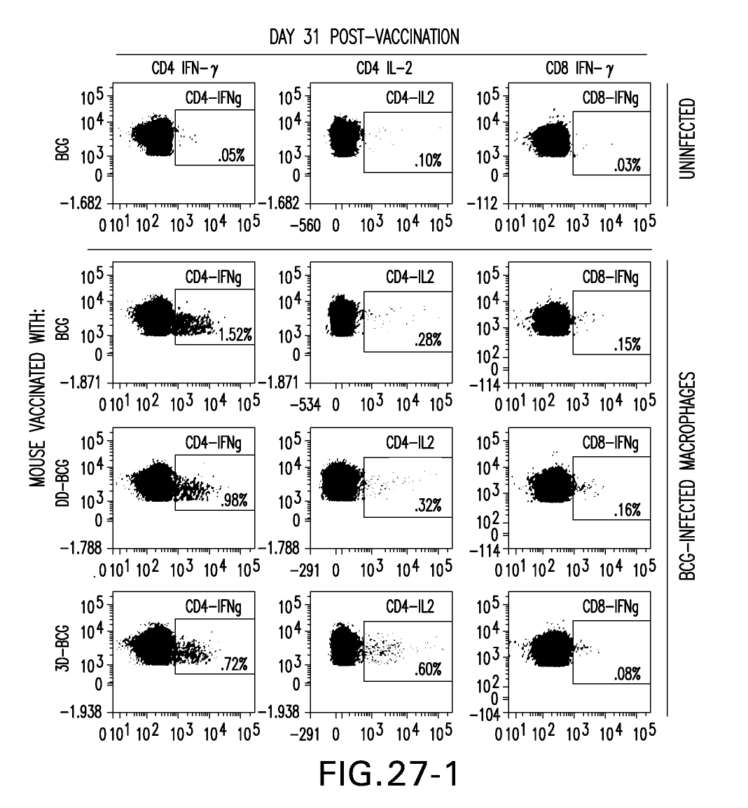

1. They induce a qualitatively and quantitatively different pattern of CD4+ T-cell responses during primary vaccination with higher peak IL-2 production and less prolonged IFN-γ release (Example 13, FIGS. 26 and 27). Both of these differences can be important in generating memory T-cells. First, IL-2 enhances the survival of antigen-specific T-cells, and is required for the generation of robust secondary responses. Second, although IFN-γ is a commonly measured effector function of effector T-cells that activates MΦs (macrophages), it promotes T-cell apoptosis during the contraction phase of primary proliferation.

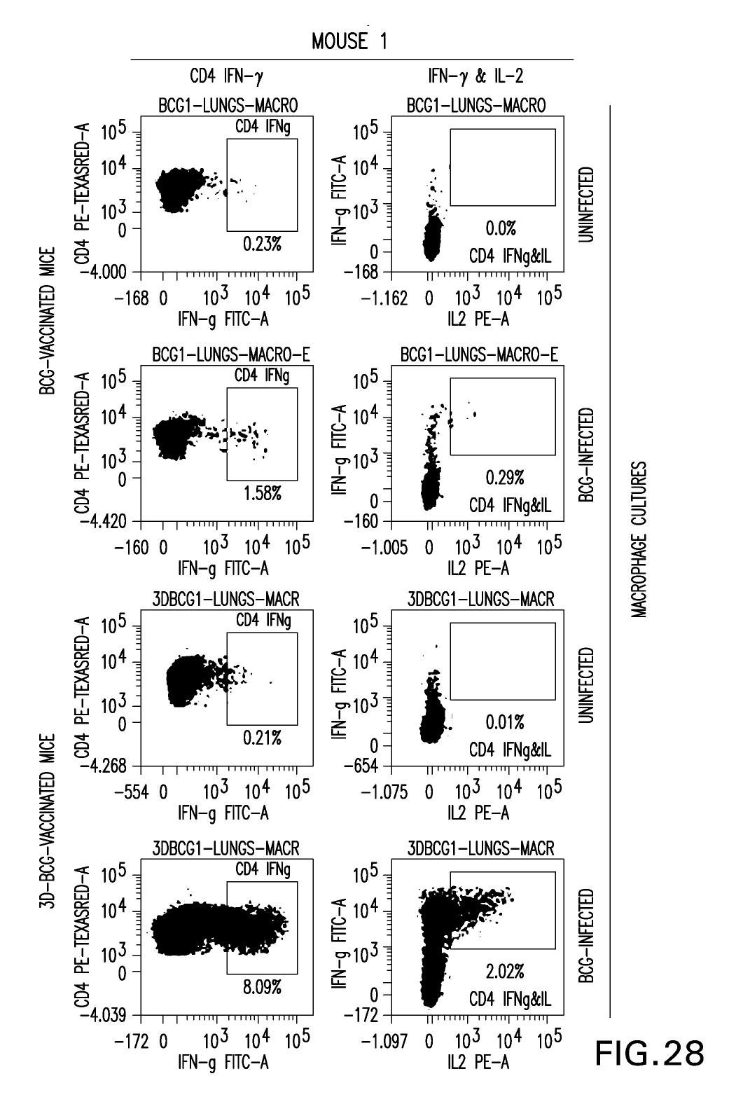

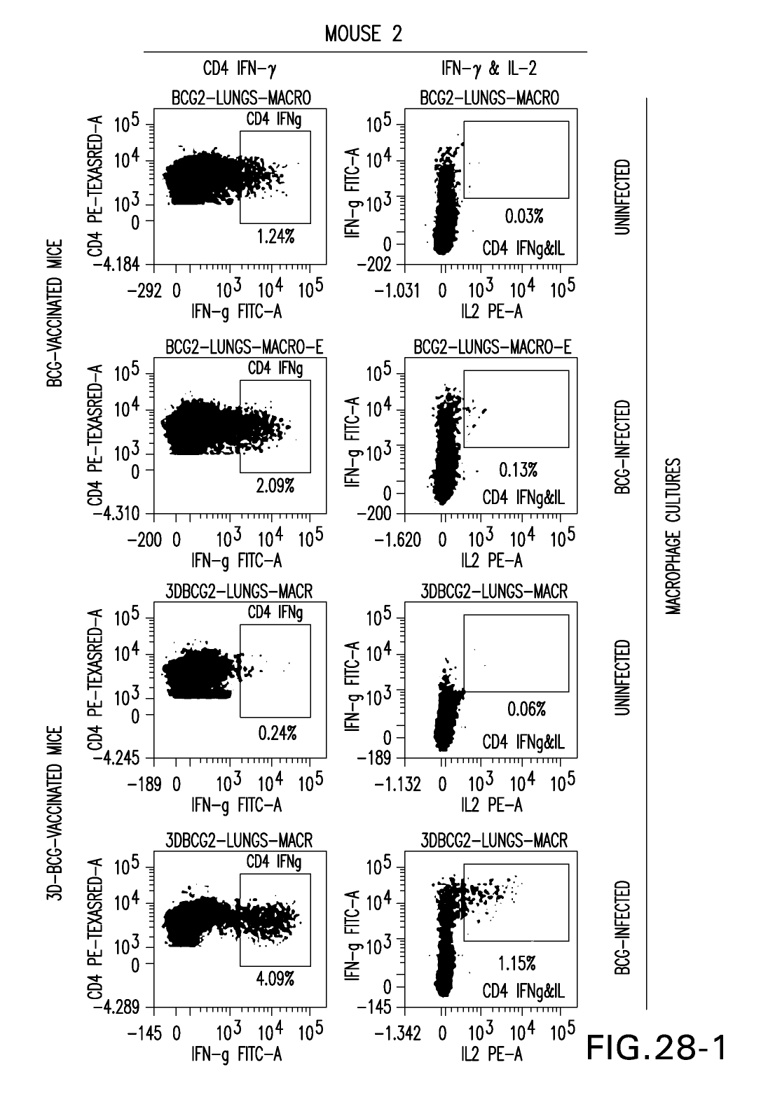

2. They induce more rapid recall T-cell responses to a second exposure. Strong T-cell responses are detected within 5 days post-challenge in mice previously subQ-vaccinated with 3DBCG (Example 14, FIG. 28). This compares favorably to recall responses in BCG-vaccinated mice which peak at day 11-14.

3. The enhancement of adaptive immune responses as outlined in (1) and (2) above appears to be due to an enhancement of innate immune responses, most notably the recruitment and activation of NK cells and PMNs within three days of administration (Example 15, FIG. 29).

### Using Pro-Apoptotic BCG Strains to Recruit and Activate Innate Immune Cells at the Site of a Cancer or Carcinoma in Situ

Many of the current uses of BCG in cancer involve the local application of BCG to the site of the tumor and are based on BCG's ability to recruit and activate cells of the innate immune response, including NK cells. Subsequently, adaptive lymphocyte responses develop and these cells are also attracted to persisting BCG bacilli in the vicinity of the tumor as well as the regional lymphatics. In the process of responding to BCG, the immune cells exhibit a bystander killing effect upon the tumor cells. As shown in the examples, paBCG induces greater recruitment and activation of NK cells and PMNs compared to the parent BCG vaccine. Thus, the modified BCG (paBCG) vaccines constructed using this technology are superior to currently-available BCG vaccines for local application including bladder cancer and intralesional injection into melanoma and other solid tumors. In effect, paBCG can replace the current BCG vaccines for already approved indications and extend the effectiveness of immunotherapy to additional cancers.

### Using Pro-Apoptotic BCG Strains as an Adjuvant

The enhanced recruitment and activation of NK cells and PMNs of paBCG compared to the parent BCG vaccine also enhances its usefulness as an adjuvant to strengthen the immune responses induced by another vaccine. In this circumstance, paBCG is typically first mixed with the other vaccine preparation, for example, a vaccine comprising a recombinant cancer antigen. Then the combined formulation is administered parenterally to a subject.

### Using Pro-Apoptotic BCG Strains to Express Exogenous Antigens

Pro-apoptotic BCG and other pro-apoptotic bacterial vaccines constructed using the dominant-negative mutant enzyme strategy, either alone or in combination with pro-apoptotic modifications of a bacterium rendered either by inactivation of a sigma factor gene, antisense techniques, or targeted incremental attenuation can be used to express exogenous antigens. The foreign DNA can be DNA from other infectious agents, for example, DNA encoding Brucella lumazine synthase (BLS), which is an immunodominant T-cell antigen from Brucella abortus. The construction of DD-BCGrBLS is described below. The foreign DNA can be DNA encoding antigens of human immunodeficiency virus (HIV), measles virus, other viruses, bacteria, fungi, or protozoan species. The foreign DNA can be a cancer antigen.

To express foreign DNA in pro-apoptotic BCG, the gene of interest is incorporated into a vector that either integrates into the chromosome of the bacterium or can be stably maintained as a plasmid within the bacterium. Methods for expressing foreign DNA in BCG and other mycobacteria have been available since 1987 [Jacobs, W. R., Jr. et al, 1987], are well-known to those skilled in the art, and include techniques taught by Bloom et al (U.S. Pat. No. 5,504,005, Recombinant mycobacterial vaccine; U.S. Pat. No. 5,854,055 and U.S. Pat. No. 6,372,478, Recombinant mycobacteria), which are hereby incorporated by reference in their entirety).

By expressing the foreign antigen in pro-apoptotic bacterial vaccines that facilitate entry into apoptosis-associated cross priming pathways of antigen presentation, the foreign antigen is introduced into this antigen presentation pathway. Furthermore, it is presented in the context of very strong co-stimulatory signals from the bacterial host that influence antigen presentation by the dendritic cells in a manner that promotes protective responses rather than the induction of tolerance. Thus, this practice enables the development of very strong adaptive T-cell responses including both CD4 and CD8 T-cells and CD4 help for CD8 T-cell responses, which has been difficult to achieve using vectors designed to access either exogenous or endogenous pathways of antigen presentation.

The present invention further provides the attenuated microbes of the invention, further expressing a heterologous antigen. The pro-apoptotic, attenuated bacteria of the present invention are optionally capable of expressing one or more heterologous antigens. As a specific example, heterologous antigens are expressed in SOD-diminished BCG bacterium of the invention. Live-attenuated vaccines have the potential to serve as vectors for the expression of heterologous antigens from other pathogenic species (Dougan et al, U.S. Pat. No. 5,980,907; Bloom et al, U.S. Pat. No. 5,504,005). Thus, the microbes of the present invention having a reduction in the expression or activity of an anti-apoptotic or essential enzyme can further be modified to express an antigen from a different microbe. Such antigens can be from viral, bacterial, protozoal or fungal microorganisms. The recombinant pro-apoptotic microorganisms then form the basis of a bi- or multivalent vaccine. In this manner, multiple pathogens can be targeted by a single vaccine strain. The invention provides a method of making a multivalent vaccine comprising transforming the pro-apoptotic microbe of the invention with a nucleic acid encoding a heterologous antigen. For example, antigens of measles virus containing immunodominant CD4+ and CD8+ epitopes can be expressed in SOD-diminished BCG, with expression achieved by stably integrating DNA encoding the measles antigen of interest into genomic DNA of the pro-apoptotic BCG of the invention using techniques taught by Bloom et al (U.S. Pat. No. 5,504,005, which is hereby incorporated by reference in its entirety). Alternatively, the gene encoding the antigen can be expressed on a plasmid vector, for example, behind the promoter of the 65 kDa heat-shock protein of pHV203 or behind an aceA(icl) promoter on any chromosomal-integration or plasmid vector using standard techniques for expressing recombinant antigens that are well-known to those skilled in the art. The antigen does not have to consist of the entire antigen but can represent peptides of a protein or glycoprotein.

A recombinant pro-apoptotic BCG vaccine expressing measles antigens can replace regular BCG as a vaccine for administration at birth in developing countries with a high incidence of infant mortality from measles. The recombinant vaccine stimulates cellular immune responses to measles antigens that protect the infant in the first few year of life when mortality from measles is the greatest. Recombinant pro-apoptotic BCG expressing measles antigens have advantages over the current live-attenuated measles vaccines, as the presence of maternal antibodies interferes with vaccination before 6 months of age, leaving the infant susceptible to measles during a period of life when they are at high risk of dying from measles. Instead, recombinant pro-apoptotic BCG expressing measles antigens are not inactivated by maternal antibodies, and can induce protective cellular immune responses at an earlier point in life. Heterologous measles virus antigens contemplated by this invention include, but are not limited to, H glycoprotein (hemagglutinin), F glycoprotein, and M protein.

Other heterologous antigens of infectious pathogens contemplated by this invention include, but are not limited to, antigens of malaria sporozoites, antigens of malaria merozoites, human immunodeficiency virus antigens, and leishmania antigens. Heterologous malaria antigens contemplated by this invention include, but are not limited to, circumsporozoite antigen, TRAP antigen, liver-stage antigens (LSA1, LSA3), blood stage molecules (MSP1, MSP2, MSP3), PfEMP1 antigen, SP166, EBA 175, AMA1, Pfs25, and Pfs45-48. Heterologous human immunodeficiency virus type 1 (HIV-1) antigens contemplated by this invention include, but are not limited to, proteins and glycoproteins encoded by env, gag, and pol including gp120, gp41, p24, p17, p7, protease, integrase, and reverse transcriptase as well as accessory gene products such as tat, rev, vif, vpr, spu, and nef. Heterologous HIV antigens include antigens from different HIV Clades. Heterologous HIV antigens also include cytotoxic T-lymphocyte (CTL) escape epitopes that are not found in native wild-type virus but which have been shown to emerge under the selective pressure of the immune system. In this manner, it vaccination can preemptively prevent mutations that enable the virus to escape from immune containment and which represents a major driving force of HIV sequence diversity. Heterologous Leishmania antigens include antigens from any Leishmania species, including but not limited to, L. donovani, L. infantum, L. chagasi, L. amazonensis, L. tropica, and L. major. Heterologous Leishmania antigens contemplated by this invention include, but are not limited to, gp63, p36(LACK), the 36-kDa nucleoside hydrolase and other components of the Fucose-Mannose-ligand (FML) antigen, glucose regulated protein 78, acidic ribosomal P0 protein, kinetoplastid membrane protein-11, cysteine proteinases type I and II, Trp-Asp (WD) protein, P4 nuclease, papLe22, TSA, LmSTI1 and LeIF.

Other heterologous antigens of infectious protozoan pathogens contemplated by this invention include, but are not limited to, antigens of Trypanosoma species, Schistosoma species, and Toxoplasma gondii. Heterologous Trypanosoma antigens include antigens from any Trypanosoma species including Trypanosoma cruzi and Trypanosoma brucei. Heterologous Trypanosoma antigens contemplated by this invention include, but are not limited to, paraflagellar rod proteins (PFR), microtubule-associate protein (MAP p15), trans-sialidase family (ts) genes ASP-1, ASP-2, and TSA-1, the 75-77-kDa parasite antigen and variable surface glycoproteins. Heterologous Schistosoma antigens include antigens from any Schistosoma species including, but not limited to, S. mansoni, S. japonicum, S. haematobium, S. mekongi, and S. intercalatum. Heterologous Schistosoma antigens contemplated by this invention include, but are not limited to, cytosolic superoxide dismutase, integral membrane protein Sm23, the large subunit of calpain (Sm-p80), triose-phosphate isomerase, filamin, paramyosin, ECL, SM14, IRV5, and Sm37-GAPDH. Heterologous Toxoplasma antigens contemplated by this invention include, but are not limited to, GRA1, GRA3, GRA4, SAG1, SAG2, SRS1, ROP2, MIC3, HSP70, HSP30, P30, and the secreted 23-kilodalton major antigen.

Other heterologous antigens of infectious viral pathogens contemplated by this invention include, but are not limited to, antigens of Influenza Virus, Hepatitis C Virus (HCV) and Flaviviruses including Yellow Fever Virus, Dengue Virus, and Japanese Encephalitis Virus. Heterologous Influenza virus antigens contemplated by this invention include, but are not limited to, the hemagglutinin (HA), neuraminidase (NA), and M protein, including different antigenic subtypes of HA and NA. Heterologous HCV antigens contemplated by this invention include, but are not limited to, the 21-kDa core (C) protein, envelope glycoproteins El and E2, and non-structural proteins NS2, NS3, NS4, and NS5. Heterologous HCV antigens include antigens from the different genotypes of HCV. Heterologous Flavivirus antigens contemplated by this invention include capsid (C) protein, envelope (E) protein, membrane (M) protein, and non-structural (NS) proteins.

Other heterologous antigens of infectious viral pathogens contemplated by this invention include, but are not limited to, structural and non-structural proteins and glycoproteins of the Herpes Virus Family including Herpes Simplex Viruses (HSV) I and 2, Cytomegalovirus (CMV), Varicella-Zoster Virus (VZV), and Epstein-Barr Virus (EBV). Heterologous herpes antigens contemplated by this invention include, but are not limited to, structural proteins and glycoproteins in the spikes, envelope, tegument, nucleocapsid, and core. Also contemplated are non-structural proteins including thymidine kinases, DNA polymerases, ribonucleotide reductases, and exonucleases.

Other heterologous antigens of infectious viral pathogens contemplated by this invention include, but are not limited to, structural and non-structural proteins and glycoproteins of Rotavirus, Parainfluenza Virus, Human Metapneumovirus, Mumps Virus, Respiratory Syncytial Virus, Rabies Virus, Alphaviruses, Hepatitis B Virus, Parvoviruses, Papillomaviruses, Variola, Hemorrhagic Fever Viruses including Marburg and Ebola, Hantaviruses, Poliovirus, Hepatitis A Virus, and Coronavirus including the agent of SARS (severe acute respiratory syndrome).

Other heterologous antigens of infectious pathogens contemplated by this invention include, but are not limited to, antigens of Chlamydia species and Mycoplasma species, including C. pneumoniae, C. psittici, C. trachomatis, M. pneumonia, and M. hyopneumoniae. Heterologous Chlamydia antigens contemplated by this invention include, but are not limited to, major outer membrane protein (MOMP), outer membrane protein A (OmpA), outer membrane protein 2 (Omp2), and pgp3. Heterologous Mycoplasma antigens contemplated by this invention include, but are not limited to, heat shock protein P42.

Other heterologous antigens of infectious pathogens contemplated by this invention include, but are not limited to, antigens of Rickettsial species including Coxiella burnetti, Rickettsia prowazekii, Rickettsia tsutsugamushi, and the Spotted Fever Group. Heterologous Rickettsial antigens contemplated by this invention include, but are not limited to, ompA, ompB, virB gene family, cap, tlyA, tlyC, the 56-1W outer membrane protein of Orientia tsutsugamushi, and the 47 kDa recombinant protein.

Other heterologous antigens of infectious pathogens contemplated by this invention include, but are not limited to, proteins and glycoproteins of bacterial pathogens including M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Treponema pallidum, other Treponema species, Leptospira species, Borrelia species, Yersinia enterolitica, and other Yersinia species.

Also, the microbes of the present invention can further be modified to express cancer antigens for use as immunotherapy against malignant neoplasms. Heterologous cancer antigens contemplated by this invention include, but are not limited to, tyrosinase, cancer-testes antigens (MAGE-1, -2, -3, -12), G-250, p53, Her-2/neu, HSP105, prostatic acid phosphatase (PAP), E6 and E7 oncoproteins of HPV16, 707 alanine proline (707-AP) (Takahashi T, et al. Clin Cancer Res. 1997 August; 3(8):1363-70); alpha (α)-fetoprotein (AFP) (Accession No. CAA79592 (amino acid), Accession No. Z19532 (nucleic acid)); adenocarcinoma antigen recognized by T cells 4 (ART-4) (Accession No. BAA86961 (amino acid), Accession No. AB026125 (nucleic acid)); B antigen (BAGE) (Accession No. NP_{—}001178 (amino acid), Accession No. NM_{—}001187 (nucleic acid)); b-catenin/mutated (Robbins P F, et al. A mutated beta-catenin gene encodes a melanoma-specific antigen recognized by tumor infiltrating lymphocytes. J Exp Med. 1996 Mar. 1; 183(3):1185-92.); breakpoint cluster region-Abelson (Bcr-abl) (Accession No. CAA10377 (amino acid), Accession No. AJ131467 (nucleic acid)); CTL-recognized antigen on melanoma (CAMEL) (Accession No. CAA10197 (amino acid), Accession No. AJ012835 (nucleic acid)); carcinoembryonic antigen peptide-1 (CAP-1) (Tsang K Y, Phenotypic stability of a cytotoxic T-cell line directed against an immunodominant epitope of human carcinoembryonic antigen. Clin Cancer Res. 1997 December; 3(12 Pt 1):2439-49); caspase-8 (CASP-8) (Accession No. NP_{—}001219 (amino acid), Accession No. NM_{—}001228 (nucleic acid)); cell-divisioncycle 27 mutated (CD27m); cycline-dependent kinase 4 mutated (CDK4/m); carcinoembryonic antigen (CEA) (Accession No. AAB59513 (amino acid), Accession No. M17303 (nucleic acid); cancer/testis (antigen) (CT); cyclophilin B (Cyp-B) (Accession No. P23284 (amino acid)); differentiation antigen melanoma (DAM) (the epitopes of DAM-6 and DAM-10 are equivalent, but the gene sequences are different) (DAM-6/MAGE-B2-Accession No. NP_{—}002355 (amino acid), Accession No. NM_{—}002364 (nucleic acid)) (DAM-10/MAGE-B1-Accession No. NP_{—}002354 (amino acid), Accession No. NM_{—}002363 (nucleic acid)); elongation factor 2 mutated (ELF2m); E-26 transforming specific (Ets) variant gene 6/acute myeloid leukemia 1 gene ETS (ETV6-AML1); glycoprotein 250 (G250); G antigen (GAGE) (Accession No. AAA82744 (amino acid));N-acetylglucosaminyltransferase V (GnT-V); glycoprotein 100 kD (Gp100); helicose antigen (HAGE); human epidermal receptor-2/neurological (HER2/neu) (Accession No. AAA58637 (amino acid) and M11730 (nucleic acid); arginine (R) to isoleucine (I) exchange at residue 170 of the α-helix of the a2-domain in the HLA-A2 gene (HLA-A*0201-R170I); human papilloma virus E7 (HPV-E7); heat shock protein 70-2 mutated (HSP70-2M); human signet ring tumor-2 (HST-2); human telomerase reverse transcriptase (hTERT or hTRT); intestinal carboxyl esterase (iCE); KIAA0205; L antigen (LAGE); low density lipid receptor/GDP-L-fucose (LDLR/FUT): b-D-galactosidase 2-a-L-fucosyltransferase; melanoma antigen (MAGE). melanoma antigen recognized by T cells-1/Melanoma antigen A (MART-1/Melan-A) (Accession No. Q16655 (amino acid) and BC014423 (nucleic acid); melanocortin 1 receptor; Myosin/m; mucin 1 (MUC1) (Acession No. CAA56734 (amino acid) X80761 (nucleic acid)); melanoma ubiquitous mutated 1, 2, 3 (MUM-1, -2, -3); NA cDNA clone of patient M88 (NA88-A); New York-esophageous 1 (NY-ESO-1); protein 15 (P15); protein of 190 KD bcr-abl; promyelocytic leukaemia/retinoic acid receptor a (Pml/RARa). preferentially expressed antigen of melanoma (PRAME) (Accession No. AAC51160 (amino acid) and U65011 (nucleic acid)); prostate-specific antigen (PSA) (Accession No. AAA58802 (amino acid) and X07730 (nucleic acid)); prostate-specific membrane antigen ((PSM) (Accession No. AAA60209 (amino acid) and AF007544 (nucleic acid)); renal antigen (RAGE) (Accesssion No. AΔH53536 (amino acid) and NM_{—}014226 (nucleic acid)); renal ubiquitous 1 or 2 (RU1 or RU2) (RU1 Accession No. AAF19794 (amino acid) and AF168132 (nucleic acid) or RU2 Accession No. AAF23610 (amino acid) AF181721 (nucleic acid)); sarcoma antigen (SAGE) (Accession No. NP_{—}005424 (amino acid) and NM_{—}018666 (nucleic acid)); squamous antigen recognized by T cells 1 or 3 (SART-1 or SART-3) (SART-1 Accession No. BAA24056 (amino acid) and NM_OO5146 (nucleic acid) or SART-3 Accession No. BAA78384 (amino acid) AB020880 (nucleic acid)); translocation Ets-family leukemia/acute myeloid leukemia 1 (TEL/AML1); triosephosphate isomerase mutated (TPI/m); tyrosinase related protein 1 (TRP-1) (Accession No. NP_{—}000541 (amino acid) and NM_{—}000550 (nucleic acid)); tyrosinase related protein 2 (TRP-2) (Accession No. CAA04137 (amino acid) and AJ000503 (nucleic acid)); TRP-2/intron 2; and Wilms' tumor gene (WT1) (Accession No. CAC39220 (amino acid) and BC032861 (nucleic acid)), which are incorporated herein by reference.

In summary, the results show that the modified BCG strains induce stronger innate and adaptive immune responses than the parent BCG vaccine.

The methods, bacterial isolates, plasmids, and other tools for performing genetic manipulations described in WO 02/062298 are hereby incorporated by reference in their entirety for the teaching of these compositions and methods.

### Using Pro-Apoptotic BCG as A General Tumor-Suppressive Agent in Malignancy

The uses of paBCG described above (i.e., local application to tumor, as an adjuvant, as a vector for expressing cancer antigens) depend largely upon the ability of the live bacterium to recruit and activate immune cells. In addition to such uses, BCG also has a general tumor-suppressive effect. Thus, there is something about active tuberculosis that suppresses the development of cancer and the administration of large doses of BCG exerts a beneficial effect upon survival in some subjects in whom cancer has already developed. Although the mechanism of benefit has not been determined, likely candidates are the Mycobacterium-induced cytokines with anti-tumor properties. In the examples below it is shown that the enhanced production of several such anti-tumor cytokines including IL-2 (FIG. 28), IL-12, and IL-21 are enhanced following administration of the paBCG vaccine 3dBCG compared to the parent BCG Tice. Thus, the paBCG vaccines constructed using this technology are superior to currently-available BCG vaccines in their ability to render a general tumor-suppressive effect.

Using Pro-Apoptotic BCG Expressing SOD-A, Mutant SOD-A, or SOD-A Peptides to Prevent the Development of Active Pulmonary Tuberculosis in Persons who already have Latent TB Infection

The rationale for active immunization against SodA of M. tuberculosis is the likelihood that it plays a central role in the conversion of latent TB infection into active pulmonary tuberculosis by promoting the expression of transferrin receptors by host cells (FIG. 29), thereby facilitating iron uptake and increasing the production of toxic oxygen derivatives that damage lung tissue (FIG. 30). Thus, immune interventions to target SodA and thereby reduce its enzymatic activity can help to prevent latent TB infection from progressing into active pulmonary tuberculosis.

Provided is a method of preventing the development of active pulmonary tuberculosis comprising immunizing a subject with a composition comprising paBCG expressing dominant-negative SodA, a composition comprising mutant SodA, or a composition comprising peptides of SodA.

Also provided is a method of reducing lung damage in persons with active pulmonary tuberculosis comprising immunizing a subject with a composition comprising paBCG expressing dominant-negative SodA, a composition comprising mutant SodA, and a composition comprising peptides of SodA.

Using Pro-Apoptotic BCG Expressing SOD-A, Mutant SOD-A, or SOD-A Peptides to Prevent the Development of Pulmonary Fibrosis in Persons Infected with Mycobacterium Species.

Mycobacterium species have also been implicated in the pathogenesis of other lung-damaging diseases including sarcoidosis. Pulmonary fibrosis was induced with histopathologic features similar to sarcoidosis by infecting C57Bl/6 mice with a saprophytic Mycobacterium species (M. vaccae) genetically engineered to express recombinant SodA (from M. tuberculosis). These results validate the understanding that Mycobacterium-derived superoxide dismutase contributes to lung damage, presumably by promoting the expression of transferrin receptors by host cells (FIG. 29) and thereby facilitating iron uptake and increasing the production of toxic oxygen derivatives (FIG. 30). Thus, immune interventions to target the SodA of Mycobacterium species and thereby reduce its enzymatic activity can help to prevent the lung fibrosing complications of sarcoidosis, and possibly other lung-fibrosing diseases including idiopathic pulmonary fibrosis (IPF).

Also provided is a method of reducing lung fibrosis in persons infected by Mycobacterium species comprising immunizing of a subject with a composition comprising paBCG expressing dominant-negative SodA, a composition comprising mutant SodA, or a composition comprising peptides of SodA.

A pharmaceutical composition comprising paBCG expressing dominant-negative SodA, a composition comprising mutant SodA, and a composition comprising peptides of SodA is also provided.

The present invention is more particularly described in the following examples which are intended as illustrative only since numerous modifications and variations therein will be apparent to those skilled in the art.

### EXAMPLES

### General Methods

Bacterial isolates, plasmids, chemicals, and culture media: Bacterial isolates and plasmids used are shown in Table 1. E. coli strain TOP 10 was used as the host for cloning PCR products and E. coli strain DH5α was used as the host for other molecular genetic manipulations unless otherwise indicated. E. coli strains were grown in LB media (Gibco/BRL, Gaithersburg, Md.). BCG Tice was grown in Middlebrook 7H9 liquid media (Difco Laboratories, Detroit, Mich.) supplemented with 0.2% glycerol, 10% Middlebrook OADC enrichment (Becton Dickinson & Co., Cockeysville, Md.), and 0.05% Tween80. Alternatively, it was grown on Middlebrook 7H10 agar (Difco) supplemented with glycerol and OADC. Kanamycin at a concentration of 50 μg/ml or 25 apramycin at a concentration of 50 μg/ml, or hygromycin at a concentration of 100 μg/ml or 50 μg/ml was used in E. coli DH5α or BCG to select for transformants containing plasmids or chromosomal integration vectors.

Gene mutagenesis. The genes for iron co-factored superoxide dismutase (sodA) and glutamine synthase (glnA1) were PCR-amplified from chromosomal DNA of M. tuberculosis strain H37RV and cloned in plasmids that replicate in E. coli. DNA sequence data stored in the TubercuList web server (http://genolist.pasteur.fr/TubercuList/site), also stored in GeneBank, was used to guide the construction of DNA primers. Site-directed mutagenesis using the PCR-based primer overlap extension methods or other methods are employed to eliminate, substitute, or add nucleotides. This produces mutant genes that encode mutant enzymes with deletions, substitutions, or additions of amino acids. Gene sequence is confirmed by DNA sequencing. Alternatively, gene synthesis techniques can be used to produce the genes with the desired sequence.

Expression of mutant enzyme genes in BCG. Genes encoding mutant enzymes were ligated into one or more of the following vectors: pMH94, pHV202, pMP349, and pMP399. Other vectors can also be used to practice this invention. Expression of mutant SodA in the chromosomal integration-proficient vector pLou1 was achieved using the cloned wild-type sodA promoter as part of an alternative strategy for practicing targeted incremental attenuation as described in WO 02/062298. This alternative strategy involved first inserting the mutant sodA allele encoding an enzyme exhibiting diminished SOD activity into the attB phage integration site on the mycobacterial chromosome. The transformants of pMH94-mut sodA grew slower than the parent BCG strain. The slow growth of these strains was similar to the slow-growth phenotype observed in M. tuberculosis and BCG strains in which antisense overexpression techniques had been used to reduce SOD activity. Realizing that this represented a dominant-negative effect of expressing the mutant SodA, the mutant SodA was then expressed in pMP349 and pMP399. In these constructs, the sodA promoter was eliminated and the mutant SodA open reading frame was placed behind a 350+ base pair region that includes the promoter for aceA (also called id). A kpn1 restriction site was used in ligation and the complete sequence of promoter-Kpn1 site-mutant SodA reading frame is shown in Example 1. The aceA promoter is macrophage-inducible and expression can also be regulated in vitro, a feature that offers potential advantages if the gene being expressed interferes with bacterial growth. Results involving mutant SodA expressed in pMP399 are shown in the examples and figures. Expression of mutant glnA1 in pMP349 and pMP399 was performed using the cloned glnA1 promoter.

The vectors were electroporated into BCG Tice using standard methods except that when the A₆₀₀ of the mycobacterial cultures reached 0.6, they were incubated in 37° C. and 5% CO₂ with 1.5% glycine and 50 ug/ml m-fluoro-DL-phenylalanine (MFP) for 48 hrs to enhance electroporation efficiency. The mycobacteria were washed twice and resuspended in ice-cold 10% glycerol. The Gene Pulser apparatus with the Pulse Controller accessory (Bio-Rad Laboratories, Hercules, Calif.) was used for all electroporations at 25F and 2.5 kV with the pulse controller set at 1000 ohms. After electroporation, 1 ml of Middlebrook 7H9 media was added to the samples, and the transformants were allowed to incubate in 37 C and 5% CO₂ for 24 hrs. Transformants were plated on Middlebrook 7H10 agar containing either kanamycin, apramycin, or hygromycin as needed. Successful transformation was confirmed by PCR of DNA unique to the vector construct.

Assays of enzyme amount and activity. The dominant-negative mutant enzyme strategy involves the expression of mutant enzyme monomers in the bacterium that interact with the bacterium's own chromosomally-encoded wild-type enzyme monomers in a manner that reduces the total activity of the enzyme produced by the bacterium. Thus, to obtain information that confirms success in the dominant-negative strategy, a non-enzymatic assay to measure enzyme quantity (e.g., Western hybridization) as well as an assay of enzyme activity were performed. The result is that compared to the parent BCG strain, the mutant BCG strains demonstrated comparable or elevated enzyme quantity (FIG. 17) but diminished enzyme activity (FIG. 16).

To prepare supernatants and lysates for enzyme activity assays, a fresh culture of each BCG strain was prepared by resuspending a washed cell pellet in 25 ml of 7119 broth containing OADC to achieve an A600 value of 0.5. Broth was grown without shaking for 72 hours. The broth culture was centrifuged and supernatant separated from the cell pellet. Concentrated supernatants for enzyme activity determinations were prepared by concentrating the 25 ml supernatant to 1.0 ml using a centrifuge-based separation device with a 10,000 kDA membrane. Lysates for testing enzyme activity were prepared by resuspending the cell pellet in 1 ml of phosphate buffered saline and lysing with a microbead-beater apparatus. Lysates from different strains were adjusted to a standard A280 value for comparison.

Western hybridization was used to quantity the amount of SOD. Samples consisting of undiluted cell lysates as prepared above were adjusted to a standard A280 values, applied to and electrophoresed on a 12% PAGE gel, and transferred to Hybond ECL nitrocellulose membranes (Amersham, Arlington Heights, Ill.). Membranes were hybridized with rabbit polyclonal antisera raised against PAGE-purified recombinant SodA expressed in E. coli. The recombinant SodA used to generate antibodies was purified by nickel affinity column chromatography. The nitrocellulose membranes were incubated first with antisera at the dilutions noted above followed by incubation with a 1:1000 dilution of horseradish peroxidase-conjugated goat anti-rabbit antibodies (Boehringer Mannheim, Indianapolis, Ind.). The immunoblots were developed with ECL Western blot detection reagents (Amersham Pharmacia, Arlington Heights, Ill.).

SOD activity was measured spectrophotometrically by its ability to interfere with the reduction of cytochrome C by superoxide using a commercial kit utilizing xanthine oxidase-generated superoxide and based on the methods of McCord and Fridovich. One SOD unit was defined as the amount of SodA that inhibited cytochrome C reduction by 50% (IC50 value).

Glutamine synthase activity was measured spectrophotometrically by using the methods of Woolfolk et al [Woolfolk, C. A. et al, 1966].

In Vivo Challenge-Protection Studies.

To prepare vaccine strain inocula for injection into C57BL/6 mice, BCG Tice and the pro-apoptotic BCG vaccine strains were grown in modified Middlebrook 71110 broth (71110 agar formulation with malachite green and agar deleted) containing 10% OADC (Difco). The suspensions were diluted to achieve a 100 Klett unit reading (approximately 5×10⁷ cfu/ml) on a Klett-Summerson Colorimeter (Klett Manufacturing, Brooklyn, N.Y.). Aliquots of the inocula were serially diluted and directly plated to 71110 agar containing 10% OADC for backcounts to determine the precise inoculum size.

Female C57BL/6 mice aged 5-6 weeks were purchased from Jackson Laboratories, Bar Harbor, Me. Infected and uninfected control mice were maintained in a pathogen-free Biosafety Level-3 facility at the Syracuse VA Medical Center. Animal experiments were approved by the Syracuse VAMC Subcommittee on Animal Studies and performed in an AALAC-approved facility.

Unless otherwise stated, the experimental design for vaccination-challenge experiments involved subcutaneous inoculation of 5×10⁶ cfu of the vaccine strain, rest for 100 days, and then challenge with an aerosol inoculum of 300 cfu of strain Erdman or acrR-Erdman. Euthanasia was achieved by CO₂ inhalation. Spleens and right lungs were removed aseptically, tissues were placed in a sealed grinding assembly (IdeaWorks! Laboratory Products, Syracuse, N.Y.) attached to a Glas-Col Homogenizer (Terre Haute, Ind.) and homogenized. Viable cell counts were determined by titration on 7H10 agar plates containing 10% OADC.

Histopathologic evaluation: Left lungs were harvested from mice and fixed in 10% formalin (Accustain, Sigma). Lungs were paraffin-embedded, cut in 4-μm sections and stained with hematoxylin and eosin.

Flow cytometry and tissue stains. Cell populations were analyzed on a Becton-Dickinson FACScalibur flow cytometer with Mac Workstation. Data were collected in listmode and offline analyses were performed using PC platform Winlist software (Verity Software House, Topsham, Me.). Antibodies for flow cytometry were purchased from BD Pharmingen (San Diego, Calif.). Samples were incubated with Rat anti-Mouse anti-CD16/CD32 clone 2.4G2 (Fc Block, BD Pharmingen) for 15 minutes to reduce background. A total of 10,000 gated events in each specimen were collected and analysis gates included a lymphocyte gate and non-lymphocyte gate based on cell size and granularity, with gate dimensions kept constant between experiments.

### Example 1

### Construction of SAD-BCG ΔH28ΔH76 [also Referred to as “BCG (mut sodA ΔH28ΔH76)”, or “SodA-Diminished BCG Expressing Dominant-Negative ΔH28ΔH76 Mutant SodA”] and Documentation of Reduced SOD Activity in Vitro

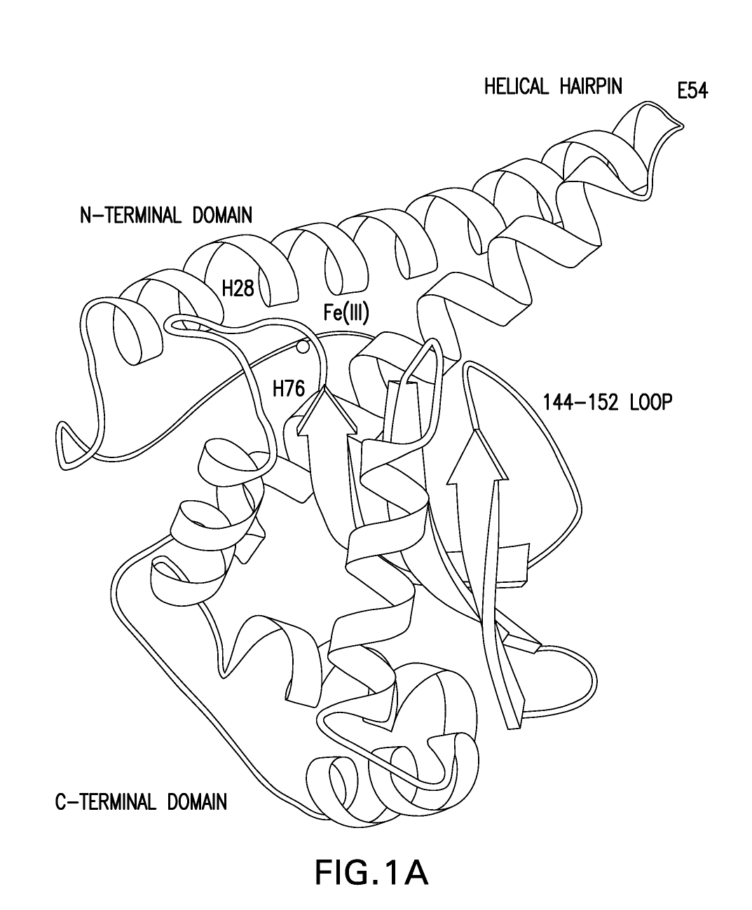

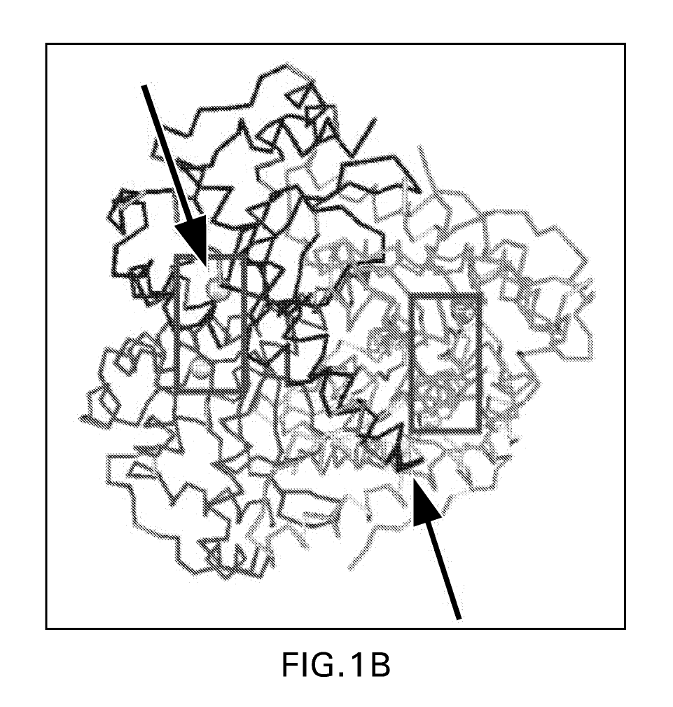

To construct SAD-BCG ΔH28ΔH76, a ΔH28ΔH76 sodA mutant in pCR2.1-TOPO was made by performing PCR-based site-directed mutagenesis on the wild-type sodA allele that had been PCR-amplified from chromosomal DNA from M. tuberculosis H37Rv. The open reading frame of the ΔH28ΔH76 mutant sodA allele is shown below. Initiation and stop codons are bold, and SEQ ID NO: 1 shows the position of the two deleted CAC (histidine-encoding) codons corresponding to amino acid 28 and amino acid 76 of the enzyme. The positions of these amino acid deletions in the context of major alpha helices, beta-strands, and the active site Fe(III) of the SodA monomer are shown in FIG. 1.

A BLASTN query of this DNA sequence against the nucleotide sequence of the complete M. tuberculosis H37Rv sequence was performed using the BLAST server of the TubercuList World Wide Web site (http://genolist.pasteur.fr/TubercuList/), documenting the deletion of the two CAC (histidine) codons. M. tuberculosis H37Rv|null M. tuberculis H37RV (4411532 bp) Identities=618/624 (99%), Gaps=6/624 (0%)

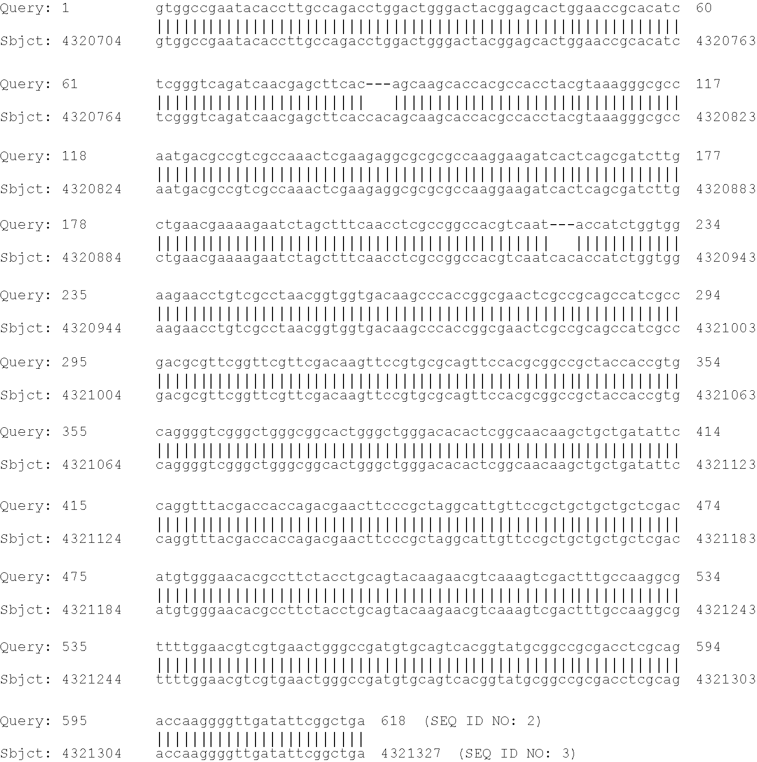

A TBLASTN query was also performed against translated nucleotide sequence data at the TubercuList BLAST site (http://genolist.pasteur.fr/TubercuList/), showing the positions of the deleted histidines. M. tuberculosis H37Rv|null M. tuberculis H37RV (4411532 bp) Identities=205/207 (99%), Positives=205/207 (99%), Gaps=2/207 (0%)

| | | | |
|---|---|---|---|
| Query: | 1 | VAEYTLPDLDWDYGALEPHISGQINELH-SKHHATYVKGANDAVAKLEEARAKEDHSAIL | 59 |
| Sbjct: | 4320704 | VAEYTLPDLDWDYGALEPHISGQINELHHSKHHATYVKGANDAVAKLEEARAKEDHSAIL | 4320883 |
| Query: | 60 | LNEKNLAFNLAGHVN-TIWWKNLSPNGGDKPTGELAAAIADAFGSFDKFRAQFHAAATTV | 118 |
| Sbjct: | 4320884 | LNEKNLAFNLAGHVNHTIWWKNLSPNGGDKPTGELAAAIADAFGSFDKFRAQFHAAATTV | 4321063 |
| Query: | 119 | QGSGWAALGWDTLGNKLLIFQVYDHQTNFPLGIVPLLLLDMWEHAFYLQYKNVKVDFAKA | 178 |
| Sbjct: | 4321064 | QGSGWAALGWDTLGNKLLIFQVYDHQTNFPLGIVPLLLLDMWEHAFYLQYKNVKVDFAKA | 4321243 |
| Query: | 179 | FWNVVNWADVQSRYAAATSQTKGLIFG | 205 (SEQ ID NO: 4) |
| Sbjct: | 4321244 | FWNVVNWADVQSRYAAATSQTKGLIFG | 4321324 (SEQ ID NO: 5) |

BLASTN and TBLASTN queries were also performed against nucleotide sequence data in the M. bovis BLAST server of the Sanger Centre (http://www.sanger.ac.uk/cgi-bin/blast/submitblast/m_{—}bovis). The Sanger Centre is sequencing Mycobacterium bovis BCG Pasteur and the preliminary M. bovis BCG assembly was used. The results (below) show that in addition to the two CAC codon deletions, in BCG there is an additional T-C nucleotide difference that yields a an I→T amino acid substitution at position 203.

BLASTN results: >BCG79c08.slk 19151 bp, 160 reads, 36.25 AT Identities=617/624 (98%), Positives=617/624 (98%), Strand=Plus/Plus

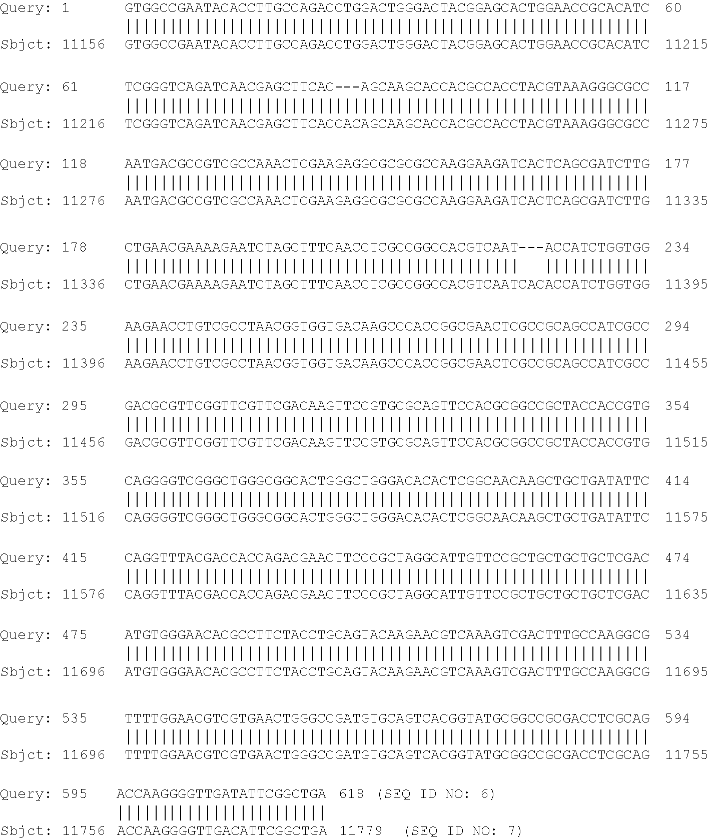

TBLASTN results: >BCG79c08.slk 19151 bp, 160 reads, 36.25 AT Identities=204/207 (98%), Positives=204/207 (98%), Frame=+2

| | | | |
|---|---|---|---|
| Query: | 1 | VAEYTLPDLDWDYGALEPHISGQINELH-SKHHATYVKGANDAVAKLEEARAKEDHSAIL | 59 |
| Sbjct: | 11156 | VAEYTLPDLDWDYGALEPHISGQINELHHSKHHATYVKGANDAVAKLEEARAKEDHSAIL | 11335 |
| Query: | 60 | LNEKNLAFNLAGHVN-TIWWKNLSPNGGDKPTGELAAAIADAFGSFDKFRAQFHAAATTV | 118 |
| Sbjct: | 11336 | LNEKNLAFNLAGHVNHTIWWKNLSPNGGDKPTGELAAAIADAFGSFDKFRAQFHAAATTV | 11515 |
| Query: | 119 | QGSGWAALGWDTLGNKLLIFQVYDHQTNFPLGIVPLLLLDMWEHAFYLQYKNVKVDFAKA | 178 |
| Sbjct: | 11516 | QGSGWAALGWDTLGNKLLIFQVYDHQTNFPLGIVPLLLLDMWEHAFYLQYKNVKVDFAKA | 11695 |
| Query: | 179 | FWNVVNWADVQSRYAAATSQTKGLIFG | 205 (SEQ ID NO: 8) |
| Sbjct: | 11696 | FWNVVNWADVQSRYAAATSQTKGLTFG | 11776 (SEQ ID NO: 9) |

Next, the mutant sodA allele was ligated into the chromosomal integration vector pMP399 and the plasmid vector pMP349 behind an aceA(icl) promoter to yield pMP399-mut SodA ΔH28ΔH76 (SEQ ID NO: 30) (Full nucleotide sequence of chromosomal integration vector pMP399-mut SodA ΔH28ΔH76 used to express the mutant sodA in BCG) and pMP349-mut SodA ΔH28ΔH76 (Table 1). It can also be added to 1^{st}, and 2^{nd}, 3^{rd} generation mutants of pro-apoptotic BCG to render, respectively, 2^{nd}, 3^{rd}, and 4^{th} generation pro-apoptotic BCG vaccines. The plasmid maps are shown in FIG. 2. The sequence shown below in SEQ ID NO: 10 highlights the nucleotide sequence of the aceA(icl) promoter through the mutant sodA open reading frame. Key features are: [a] the sequence encoding the aceA(icl)-associated promoter (base 5044 to base 5385, [b] the open reading frame for the sodA(ΔH28ΔH76) mutant (base 9-base 626), and [c] a Kpn1 restriction site (base 1-base 8) used to connect [a] and [b]:

Next, pMP399-mut SodA ΔH28ΔH76 was electroporated into BCG Tice to produce SAD-BCG ΔH8ΔH76 (SodA-Diminished BCG, also called BCG (mut sodA ΔH28ΔH76). Transformants were selected on agar containing apramycin. PCR of chromosomal DNA using nucleotide sequences unique to the pMP399 vector was used to verify successful integration of the vector into the BCG chromosome.

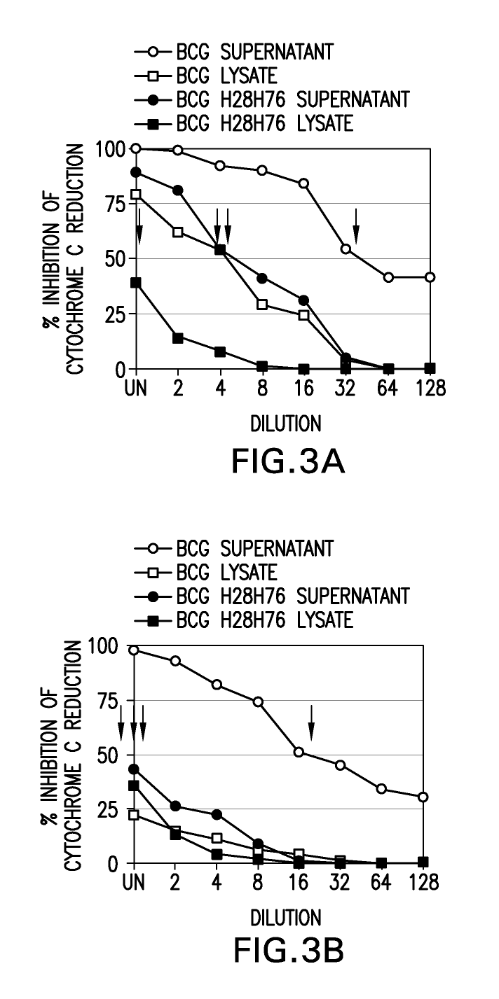

To determine the effect of expressing mutant ΔH28ΔH76 SodA upon the SOD activity of the whole bacterium, supernatants and lysates of BCG and SAD-BCG ΔH28ΔH76 were prepared as described above and compared for SOD activity by monitoring interference (by SOD) with reduction of cytochrome C by xanthine oxidase-generated superoxide (O2-). Results are shown in FIG. 3 and demonstrate that most of the activity can be found in the supernatant, and that the dominant-negative strategy results in an approximately 8- to 16-fold reduction in SOD activity.

### Example 2

### Construction of SAD-BCG ΔE54 [aka BCG (mut sodA ΔE54), or SodA-Diminished BCG Expressing Dominant-Negative ΔE54 Mutant SodA] and Documentation of Reduced SOD Activity in vitro

An additional dominant-negative sodA mutant with a ΔE54 deletion was constructed using the techniques described. The position of this amino acid deletion in the context of major alpha helices, beta-strands, and the active site Fe(III) of the SodA monomer are shown in FIG. 1. DNA sequencing of the gene in pCR2.1-TOPO identified an additional nucleotide substitution that introduced a histidine→arginine substitution at position 28.

The mutant ΔE54 sodA allele was ligated into the chromosomal integration vector pMP399 and the plasmid vector pMP349 behind an aceA(icl) promoter to yield pMP399-mut SodA ΔE54 (SEQ ID NO: 29) and pMP349-mut SodA ΔE54 (SEQ ID NO: 24) (Table 1). pMP399-mut SodA ΔE54 was electroporated into BCG Tice to produce SAD-BCG ΔE54 (SodA-Diminished BCG, also called BCG (mut sodA ΔE54). These vectors can also be added to 1^{st}, and 2^{nd}, and 3^{rd} generation mutants of pro-apoptotic BCG to construct, respectively, 2^{nd}, 3^{rd}, and 4^{th} generation pro-apoptotic BCG vaccines

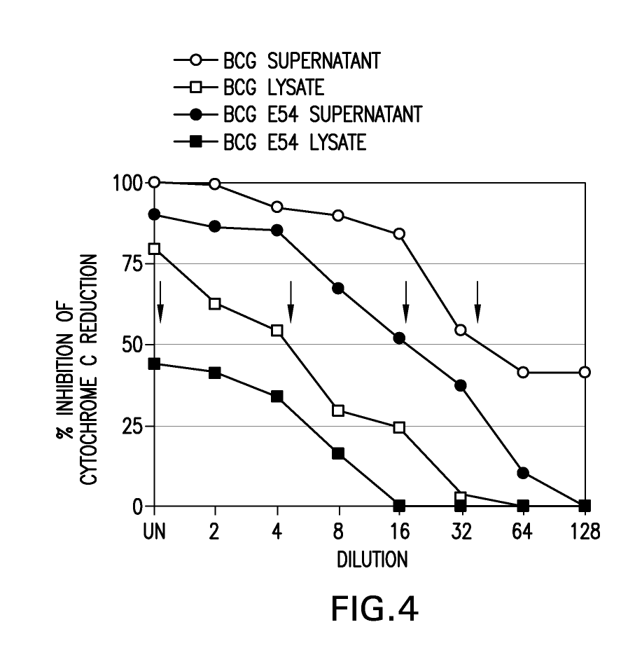

To determine the effect of expressing mutant ΔE54 SodA upon the SOD activity of the whole bacterium, supernatants and lysates of BCG and SAD-BCG ΔE54 were prepared as described above and compared for SOD activity. Results are shown in FIG. 4 and demonstrate a less marked reduction in total SOD activity than was observed with SAD-BCG ΔH28ΔH76.

### Example 3

### The Vaccine Efficacy of SD-BCG-AS-SOD—Implications Regarding the Usefulness of Dominant-Negative SodA-Diminished BCG Strains

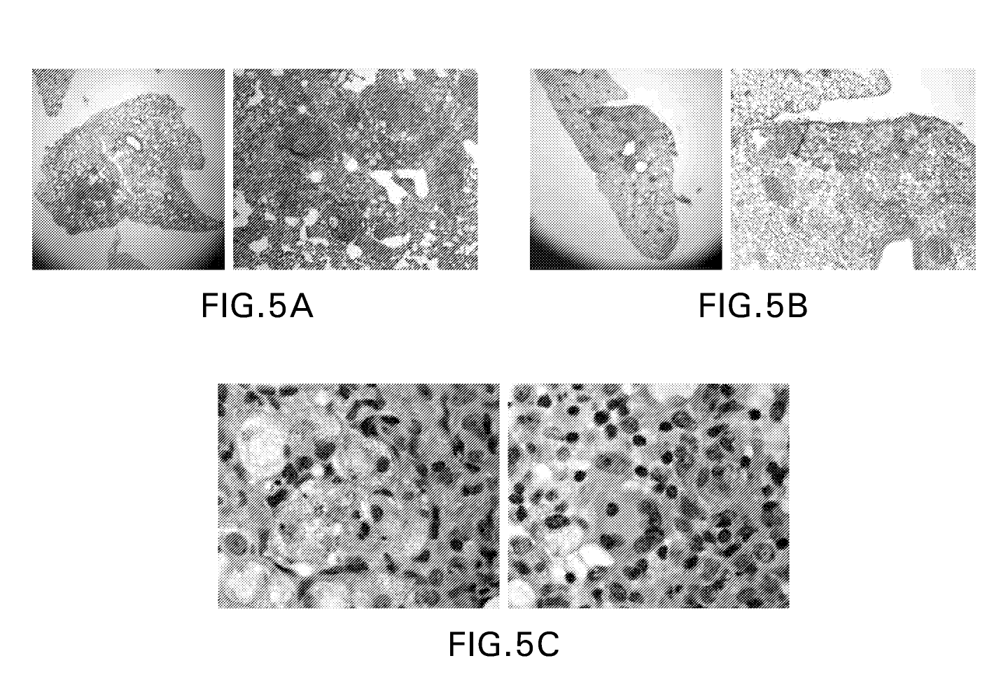

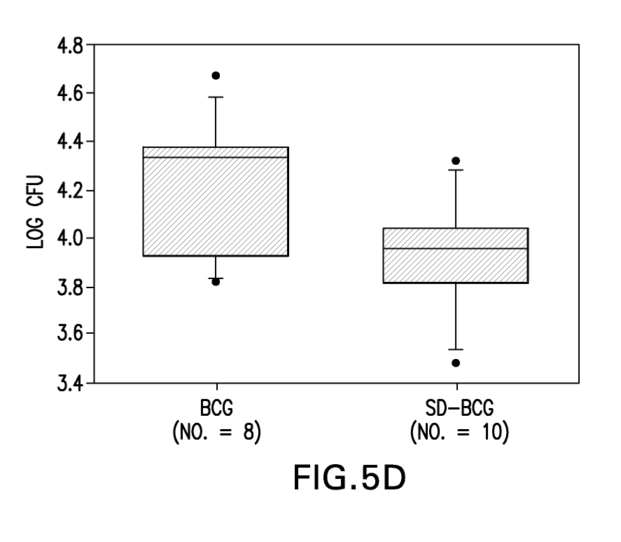

To quantify the amount of improvement in vaccine efficacy that occurs as a consequence of reducing SodA production by BCG, BCG and SD-BCG-AS-SOD (SodA-diminished BCG constructed by using antisense techniques as previously described in WO 02/062298) were compared. Experimental details and results are shown in FIG. 5 and indicate that C57Bl/6 mice vaccinated with SD-BCG-AS-SOD had lower lung cfu counts and less lung damage than mice vaccinated with BCG at six months following aerosol challenge with virulent M. tuberculosis.

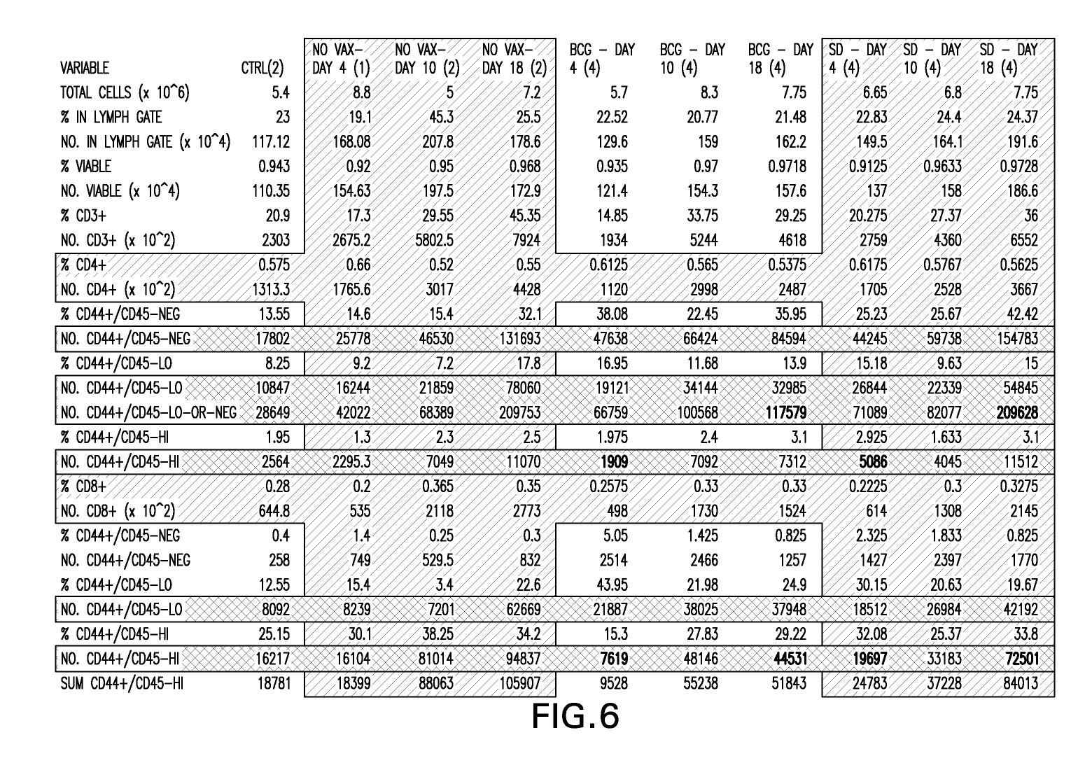

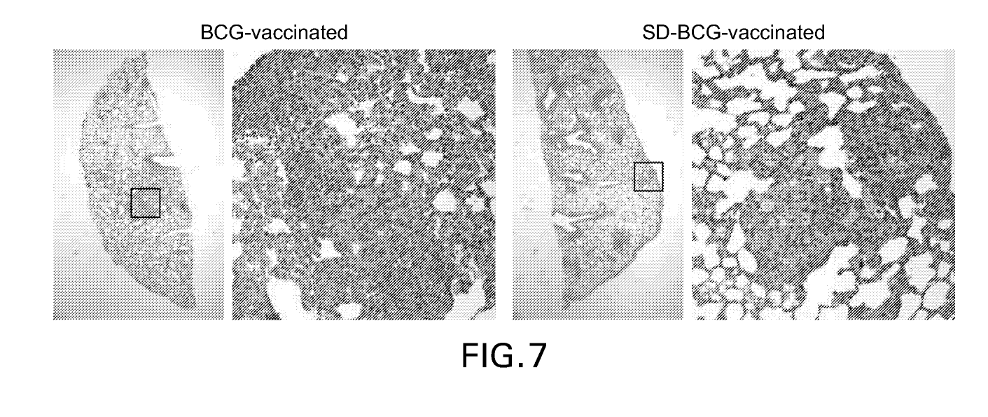

In a separate vaccination-challenge experiment, C57Bl/6 mice were vaccinated subcutaneously, rested for 100 days, and harvested for analysis of T-cell responses in the lung at 4, 10, and 18 days post-aerosol challenge with virulent M. tuberculosis. Compared to mice vaccinated with BCG, mice vaccinated with SD-BCG-AS-SOD exhibited greater numbers of CD4+ and CD8+ T-cells that were CD44+/CD45RBhigh at 4 days post-challenge, and greater numbers of CD4+ T-cells that were CD44+/CD45RBneg at 18 days (FIG. 6). These differences in T-cell responses were associated with a difference in the histopathologic appearance of the lungs early post-challenge including the more rapid development of Ghon lesions (FIG. 7).

Based on these results and results reported elsewhere herein, comparable enhancement of vaccine efficacy occurs with the SAD-BCG strains constructed by using dominant-negative mutant SodA expression as described above.

### Example 4

### Construction and Vaccine Evaluation of SIG-BCG (also Referred to as: BCGΔsigH)

The effect of diminishing other antioxidants produced by BCG upon vaccine efficacy was assessed. As discussed above, sigH is a sigma factor implicated in the bacterial response to oxidative stress and regulates the production of thioredoxin, thioredoxin reductase, and a glutaredoxin homolog.

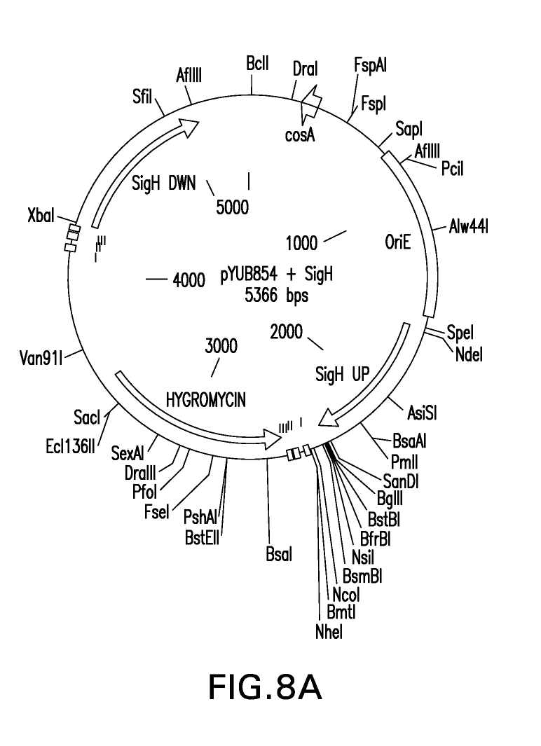

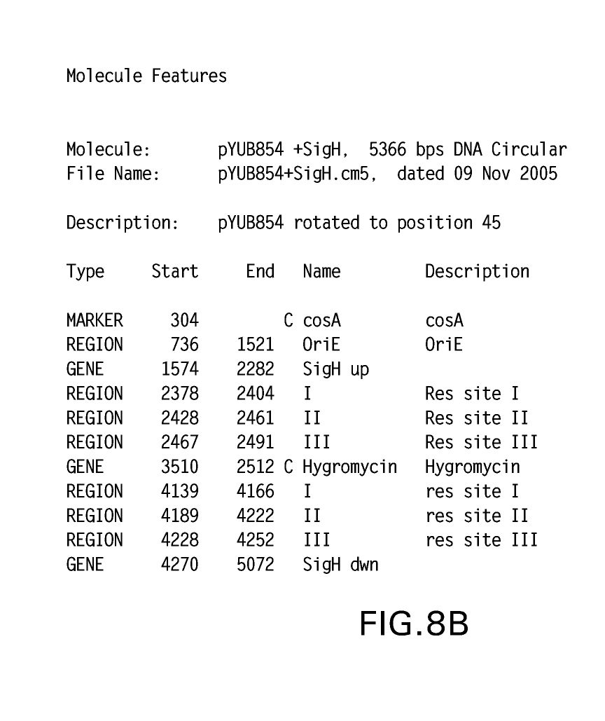

SigH on the chromosome of BCG Tice was inactivated by using the phasmid system of William Jacobs, Jr. from Albert Einstein College of Medicine, using published methods for applying this system to inactivate genes in mycobacteria. Upstream and downstream regions of sigH were cloned into pYUB854 to construct the allelic inactivation vector—the DNA sequence of pYUB854-sigH is shown in the SEQ ID NO: 34 and the map and features of this vector are shown in FIG. 8. The vector for sigH inactivation by using the phasmid system, added to BCG to construct BCGΔsigH and to BCGΔsecA2 to construct DD-BCG. It can be used to modify 1^{st}, 2^{nd}, and 3^{rd} generation pro-apoptotic BCG vaccines, respectively, into 2^{nd}, 3^{rd}, and 4^{th} generation pro-apoptotic BCG vaccines.

An alternative strategy for constructing SIG-BCG (BCGΔsigH) involves the use of suicide plasmid vectors as described and referenced above, the use of which are well-known among those skilled in the art.

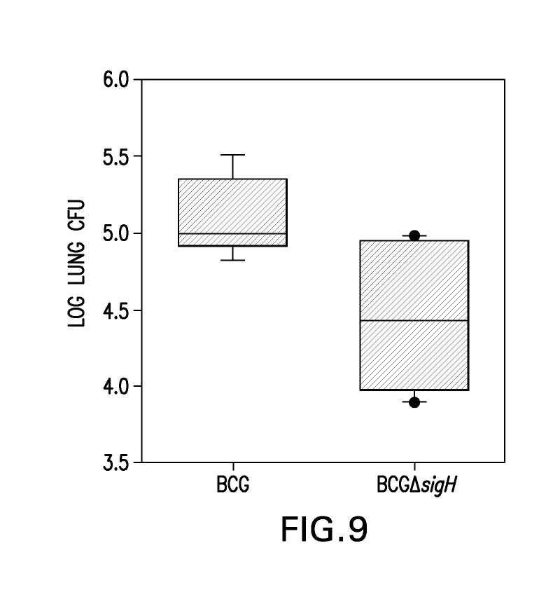

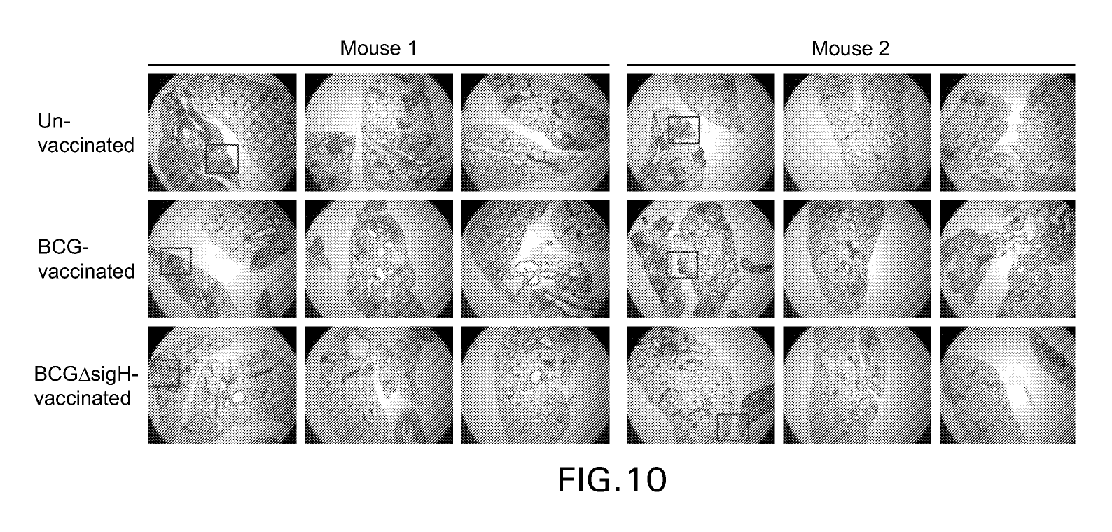

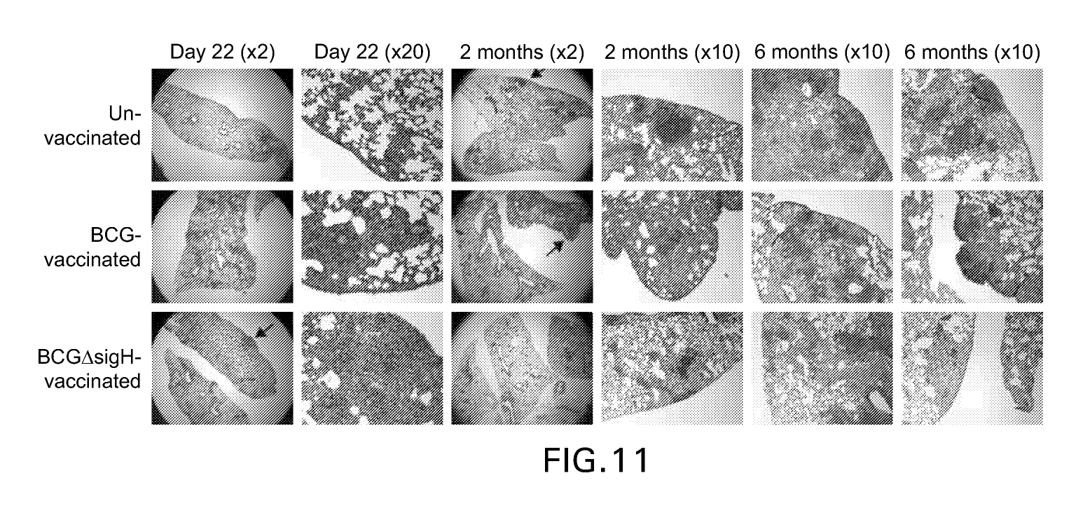

SIG-BCG was tested as a vaccine. C57Bl/6 mice were vaccinated subcutaneously with either BCG or SIG-BCG, rested for 100 days, and then challenged by aerosol with the AcrR-Erdman strain of virulent M. tuberculosis. At six months post-challenge, mice vaccinated with SIG-BCG had lower lung cfu counts of virulent M. tuberculosis (FIG. 9) and less lung damage (FIG. 10) than mice vaccinated with BCG. The histopathologic appearance over time of the lungs of SIG-BCG-vaccinated mice challenged with virulent M. tuberculosis showed similarities to results shown above for mice vaccinated with SD-BCG-AS-SOD (example 4)—most notable were the earlier development of Ghon lesions in mice vaccinated with SIG-BCG and their apparent resolution over time (FIG. 11) that corresponded with the lower lung cfu counts.

### Example 5

### Construction of SAD-SIG-BCG, a “Second-Generation Pro-Apoptotic BCG Vaccine”, and Documentation of Reduced SOD Activity in vitro

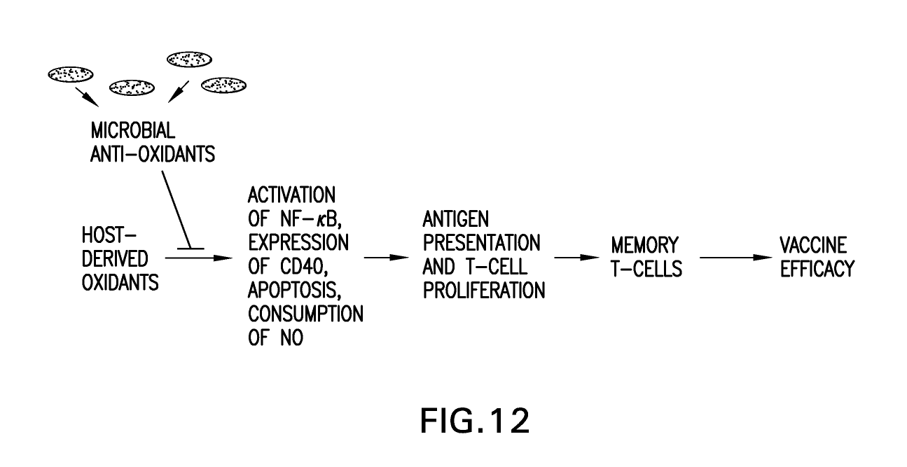

The increased vaccine efficacy of two different pro-apoptotic BCG vaccines (SD-BCG-AS-SOD and SIG-BCG) as exemplified in examples 3 and 4 shows that host-generated oxidants have important functions in the host immune response. Microbial anti-oxidants interfere with these important functions of oxidants (FIG. 12) and thereby disrupt the early signaling needed to develop a strong protective immune response.

The observations of examples 3 and 4 involving two pro-apoptotic BCG vaccines, each with a single genetic modification, indicate that introducing two or more defects in antioxidant production by BCG yields a more potent vaccine. For Example, introducing defects in antioxidant production by BCG increases BCG's ability to protect against pulmonary tuberculosis. As discussed above, microorganisms produce a diverse array of anti-apoptotic enzymes, many of which are involved in inactivating host oxidants. FIG. 13 shows a strategy for combining genetic modifications in BCG (and M. tuberculosis) to introduce one, two, three, or four genetic manipulations that reduce antioxidant production, yielding respectively, 1st, 2nd, 3rd, and 4th generation pro-apoptotic vaccines.

To produce “2nd generation” pro-apoptotic BCG vaccines, dominant-negative mutant sodA expression vectors (pMP399-mut SodA ΔH28ΔH76 (SEQ ID NO: 30); pMP349-mut SodA ΔH28ΔH76 (SEQ ID NO: 25); pMP399-mut SodA ΔE54 (SEQ ID NO: 29); and pMP349-mut SodA ΔE54 (SEQ ID NO: 24)) were electroporated into SIG-BCG to yield SAD-SIG-BCG. The results of SOD activity assays on lysates and supernatants of these strains are shown in FIG. 14 and demonstrate similar reductions in SOD activity to those shown with the 1st generation SAD-BCG vaccines. Overexpression of the dominant-negative ΔH28ΔH76 sodA mutant resulted in greater reduction in SOD activity (about 8-fold) than overexpression of the ΔE54 sodA mutant (about 4-fold).

### Example 6

### Construction of DD-BCG (also Referred to as: BCGΔsigHΔsecA2)

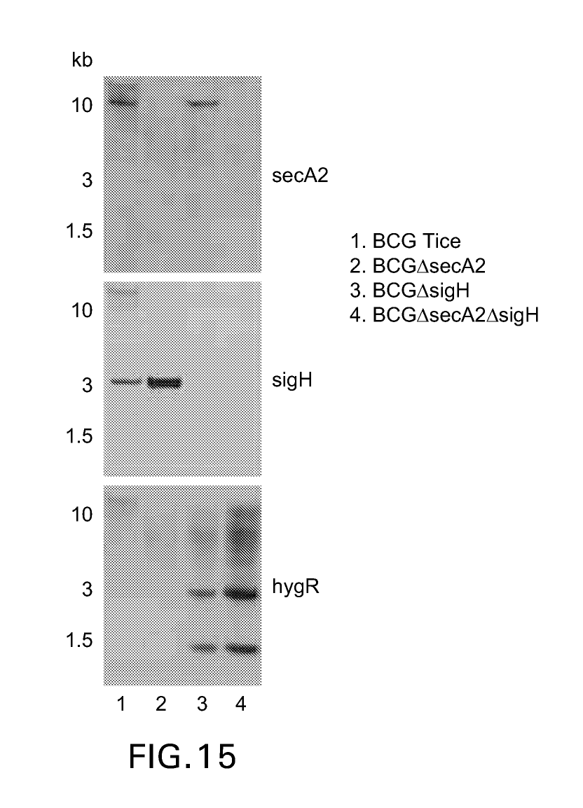

Another “2nd generation” pro-apoptotic BCG vaccine was produced by using the methods outlined in example 4 to inactivate sigH on the chromosome of SEC-BCG (also referred to as: “BCGΔsecA2”) to produce DD-BCG, which is an abbreviation of “double-deletion BCG”. FIG. 15 shows a Southern hybridization membrane that documents the successful construction of DD-BCG. DD-BCG comprises inactivated secA2 and sigH.

### Example 7

### Construction of 3D-BCG and Documentation of Reduced SOD Activity in vitro

To produce “3rd generation” pro-apoptotic BCG vaccines, dominant-negative mutant sodA expression vectors (pMP399-mut SodA ΔH28ΔH76; pMP349-mut SodA ΔH28ΔH76; pMP399-mut SodA ΔE54; and pMP349-mut SodA ΔE54) were electroporated into DD-BCG to yield 3D-BCG.

The results of SOD activity assays on lysates and supernatants of these strains are shown in FIG. 16. In contrast to results involving SAD-BCG and SAD-SIG-BCG in which the SOD activity was predominantly in the supernatant (FIGS. 3, 4, 14), the results in FIG. 16A show that the SOD activity in DD-BCG and 3D-BCG is predominantly in the cell lysates. This reversal occurs because the inactivation of secA2 in BCG disrupts the secretion channel for SodA, causing it to be withheld by the bacterium rather than secreted extracellularly.

This localization of SodA in the lysates of these strains facilitated the use of other techniques to quantify the amount of SodA. FIG. 17 shows SDS-PAGE and Western hybridization results comparing the amount of SodA as determined by direct observation of the 23-kDa SodA band on SDS-PAGE and after hybridization with rabbit polyclonal anti-SodA antibody (Western). These results indicate that despite the marked reduction in SOD activity exhibited by 3D-BCG isolates in which the ΔH28ΔH76 and ΔE54 SodA mutants have been overexpressed, there is a comparable amount of SodA protein. This indicates that the overexpression of ΔH28ΔH76 and ΔE54 SodA mutants induces a dominant-negative effect, interfering with the biological activity of SodA despite comparable amounts of total (wild-type plus mutant) SodA protein.

These results also indicate that there can be an advantage of practicing the dominant-negative mutant SodA strategy in combination with allelic inactivation of secA2. There appears to be a greater overall reduction in total SOD activity in strains with the secA2 deletion compared to strains without this deletion. For example, whereas SAD-BCG and SAD-SIG-BCG isolates with overexpressed dominant-negative ΔH28ΔH76 SodA mutant exhibited an 8- to 16-fold reduction in total SOD activity (FIGS. 3, 14), the reduction appeared to be 32-fold or greater when the ΔH28ΔH76 SodA mutant was added to DD-BCG (FIG. 16). Similarly, a greater reduction in SOD activity was achieved when the ΔE54 SodA mutant was put into DD-BCG (FIG. 16; 16-fold reduction) than in BCG or SIG-BCG (FIGS. 4, 14; 2- to 4-fold reduction).

### Example 8

### Addition of Dominant-Negative Glutamine Synthase to 3D-BCG to Yield 4D-BCG Vaccines

Glutamate and glutamine exert pro- and anti-apoptotic effects, respectively, upon mammalian cells. Glutamine synthase (also called “glutamine synthetase”) catalyzes the reaction between glutamate and ammonia to yield glutamine. M. tuberculosis and BCG have several alleles on their chromosome that encode glutamine synthase or homologs. One of these, glnA1, is produced in large amounts and secreted extracellularly.

To construct 4D-BCG, a dominant-negative glnA1 mutant in pCR2.1-TOPO was constructed by performing PCR-based site-directed mutagenesis on the wild-type glnA1 allele that had been PCR-amplified from chromosomal DNA from M. tuberculosis H37Rv. The open reading frame of the ΔD54ΔE335 mutant glnA1 allele is shown below. Initiation and stop codons are bold, and SEQ ID NO: 11 shows the position of the two deleted codons corresponding to amino acid 54 and amino acid 335 of the enzyme.

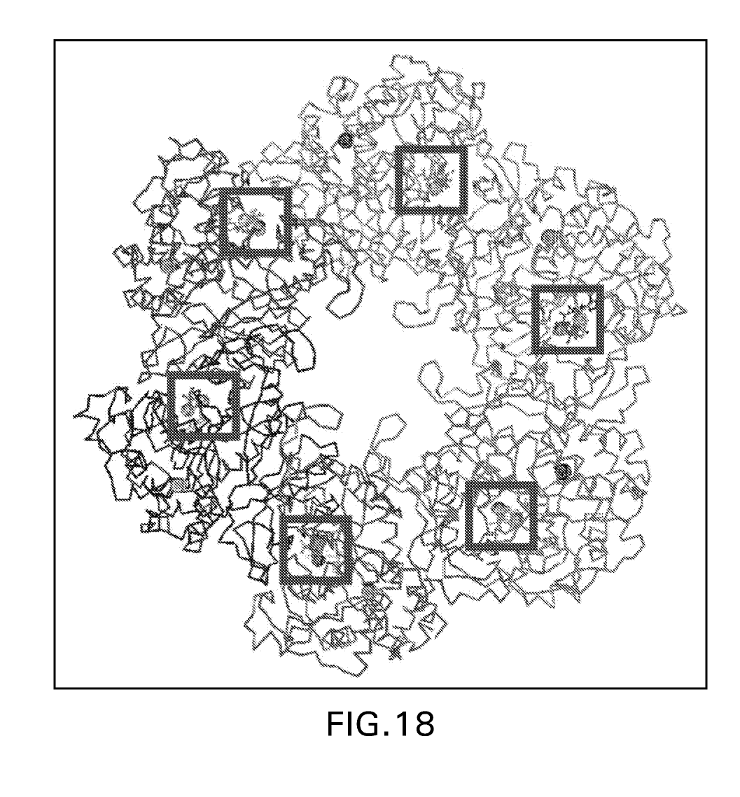

The positions of these amino acid deletions in the context of the active-site manganese ions of the hexameric glnA1 ring are shown in FIG. 18. As the D54 and E335 from adjacent monomers are involved in forming the active sites, which lie between monomers, introducing both deletions in a single monomer disrupts the active sites on each side of the monomer as it assembles into rings with wild-type monomers. Thus, it induces a dominant-negative effect.

A BLASTN query of this DNA sequence against the nucleotide sequence of the complete M. tuberculosis H37Rv sequence was performed using the BLAST server of the TubercuList World Wide Web site (http://genolist.pasteur.fr/TubercuList/), documenting the deletion of the two codons.

M. tuberculosis H37Rv|null M. tuberculis H37RV (4411532 bp) Identities=1431/1437 (99%), Gaps=6/1437 (0%)

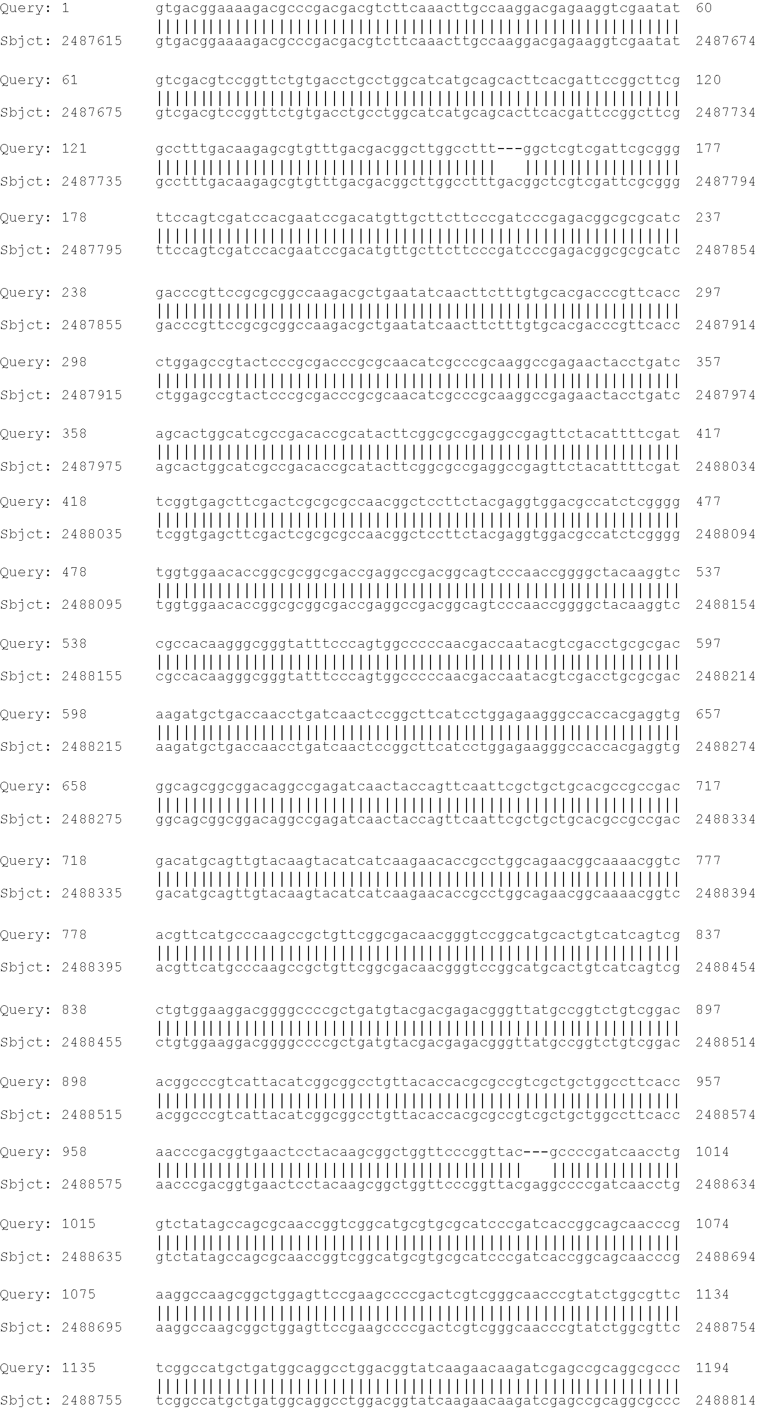

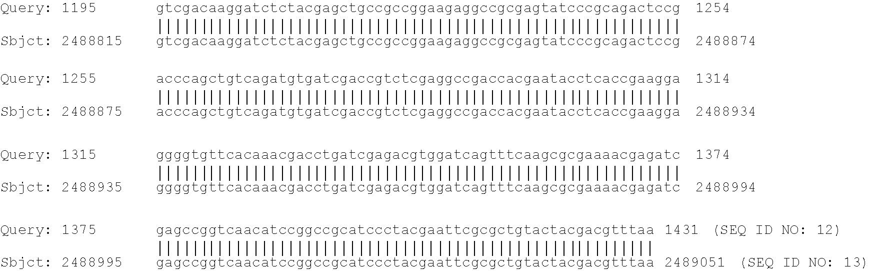

A TBLASTN query was also performed against translated nucleotide sequence data at the TubercuList BLAST site (http://genolist.pasteur.fr/TubercuList/), showing the positions of the deleted aspartic acid and glutamic acid.

M. tuberculosis H37Rv|null M. tuberculis H37RV (4411532 bp) Identities=476/478 (99%), Positives=476/478 (99%), Gaps=2/478 (0%)

| | | | |
|---|---|---|---|
| Query: | 1 | VTEKTPDDVFKLAKDEKVEYVDVRFCDLPGIMQHFTIPASAFDKSVFDDGLAF-GSSIRG | 59 |
| | | VTEKTPDDVFKLAKDEKVEYVDVRFCDLPGIMQHFTIPASAFDKSVFDDGLAF GSSIRG | |
| Sbjct: | 2487615 | VTEKTPDDVFKLAKDEKVEYVDVRFCDLPGIMQHFTIPASAFDKSVFDDGLAFDGSSIRG | 2487794 |
| Query: | 60 | FQSIHESDMLLLPDPETARIDPFRAAKTLNINFFVHDPFTLEPYSRDPRNIARKAENYLI | 119 |
| | | FQSIHESDMLLLPDPETARIDPFRAAKTLNINFFVHDPFTLEPYSRDPRNIARKAENYLI | |
| Sbjct: | 2487795 | FQSIHESDMLLLPDPETARIDPFRAAKTLNINFFVHDPFTLEPYSRDPRNIARKAENYLI | 2487974 |
| Query: | 120 | STGIADTAYFGAEAEFYIFDSVSFDSRANGSFYEVDAISGWWNTGAATEADGSPNRGYKV | 179 |
| | | STGIADTAYFGAEAEFYIFDSVSFDSRANGSFYEVDAISGWWNTGAATEADGSPNRGYKV | |
| Sbjct: | 2487975 | STGIADTAYFGAEAEFYIFDSVSFDSRANGSFYEVDAISGWWNTGAATEADGSPNRGYKV | 2488154 |
| Query: | 180 | RHKGGYFPVAPNDQYVDLRDKMLTNLINSGFILEKGHHEVGSGGQAEINYQFNSLLHAAD | 239 |
| | | RHKGGYFPVAPNDQYVDLRDKMLTNLINSGFILEKGHHEVGSGGQAEINYQFNSLLHAAD | |
| Sbjct: | 2488155 | RHKGGYFPVAPNDQYVDLRDKMLTNLINSGFILEKGHHEVGSGGQAEINYQFNSLLHAAD | 2488334 |
| Query: | 240 | DMQLYKYIIKNTAWQNGKTVTFMPKPLFGDNGSGMHCHQSLWKDGAPLMYDETGYAGLSD | 299 |
| | | DMQLYKYIIKNTAWQNGKTVTFMPKPLFGDNGSGMHCHQSLWKDGAPLMYDETGYAGLSD | |
| Sbjct: | 2488335 | DMQLYKYIIKNTAWQNGKTVTFMPKPLFGDNGSGMHCHQSLWKDGAPLMYDETGYAGLSD | 2488514 |
| Query: | 300 | TARHYIGGLLHHAPSLLAFTNPTVNSYKRLVPGY-APINLVYSQRNRSACVRIPITGSNP | 358 |
| | | TARHYIGGLLHHAPSLLAFTNPTVNSYKRLVPGY APINLVYSQRNRSACVRIPITGSNP | |
| Sbjct: | 2488515 | TARHYIGGLLHHAPSLLAFTNPTVNSYKRLVPGYEAPINLVYSQRNRSACVRIPITGSNP | 2488694 |
| Query: | 359 | KAKRLEFRSPDSSGNPYLAFSAMLMAGLDGIKNKIEPQAPVDKDLYELPPEEAASIPQTP | 418 |
| | | KAKRLEFRSPDSSGNPYLAFSAMLMAGLDGIKNKIEPQAPVDKDLYELPPEEAASIPQTP | |
| Sbjct: | 2488695 | KAKRLEFRSPDSSGNPYLAFSAMLMAGLDGIKNKIEPQAPVDKDLYELPPEEAASIPQTP | 2488874 |
| Query: | 419 | TQLSDVIDRLEADHEYLTEGGVFTNDLIETWISFKRENEIEPVNIRPHPYEFALYYDV | 476 (SEQ ID NO: 14) |
| | | TQLSDVIDRLEADHEYLTEGGVFTNDLIETWISFKRENEIEPVNIRPHPYEFALYYDV | (SEQ ID NO: 39) |
| Sbjct: | 2488875 | TQLSDVIDRLEADHEYLTEGGVFTNDLIETWISFKRENEIEPVNIRPHPYEFALYYDV | 2489048 (SEQ ID NO: 15) |

BLASTN and TBLASTN queries were also performed against nucleotide sequence data in the M. bovis BLAST server of the Sanger Centre (http://www.sanger.ac.uk/cgi-bin/blast/submitblast/m_{—}bovis). The Sanger Centre is sequencing Mycobacterium bovis BCG Pasteur the preliminary M. bovis BCG assembly was used. The results show that the glnA1 nucleotide sequence in BCG Pasteur is identical to the glnA1 nucleotide sequence in M. tuberculosis H37Rv.

BLASTN results: BCG260c11.qlk 3891 bp, 23 reads, 35.42 AT Identities=1431/1437 (99%), Positives=1431/1437 (99%), Strand=Minus/Plus

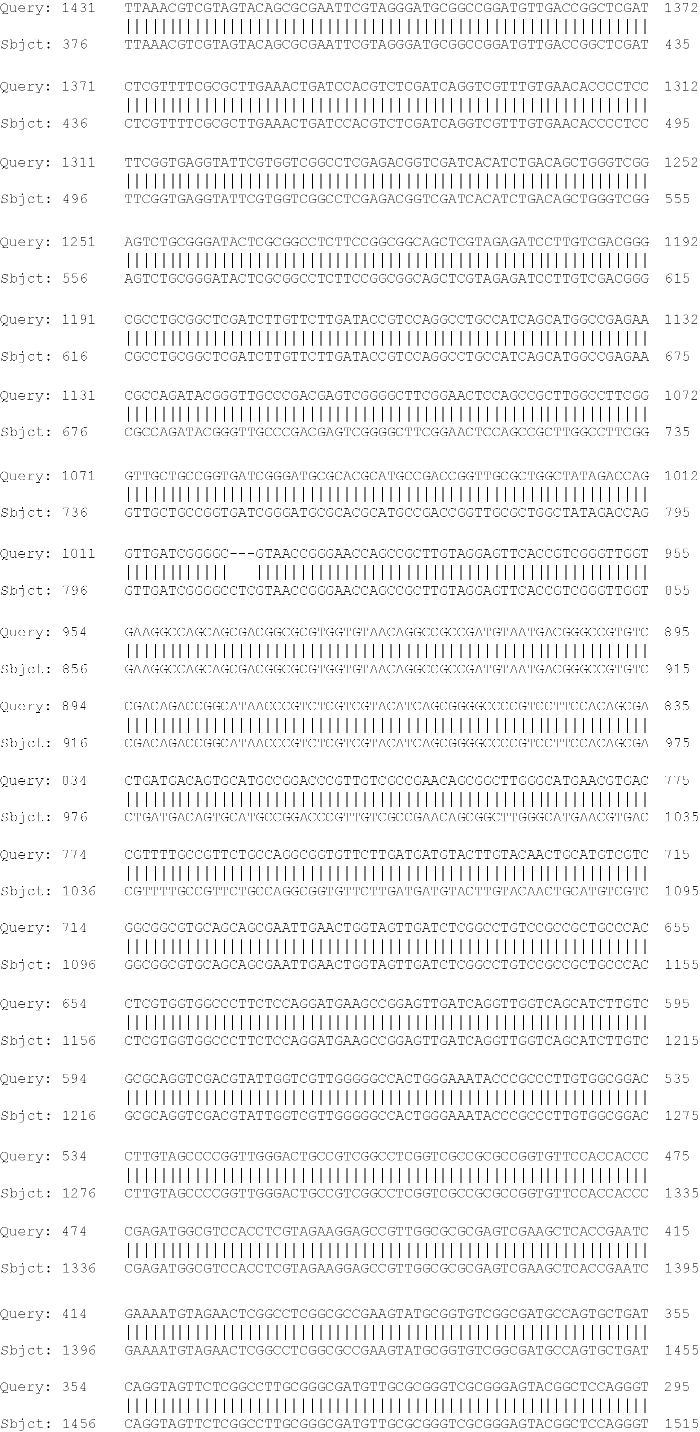

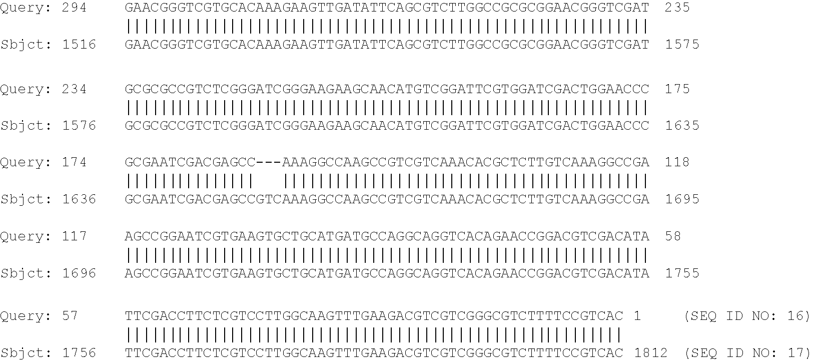

TBLASTN results: BCG260c11.qlk 3891 bp, 23 reads, 35.42 AT Identities=476/478 (99%), Positives=476/478 (99%), Frame=−1

| | | | |
|---|---|---|---|
| Query: | 1 | VTEKTPDDVFKLAKDEKVEYVDVRFCDLPGIMQHFTIPASAFDKSVFDDGLAF-GSSIRG | 59 |
| | | VTEKTPDDVFKLAKDEKVEYVDVRECDLPGIMQHFTIPASAFDKSVFDDGLAF GSSIRG | |
| Sbjct: | 1812 | VTEKTPDDVFKLAKDEKVEYVDVRFCDLPGIMQHFTIPASAFDKSVFDDGLAFDGSSIRG | 1633 |
| Query: | 60 | FQSIHESDMLLLPDPETARIDPFRAAKTLNINFFVHDPFTLEPYSRDPRNIARKAENYLI | 119 |
| | | FQSIHESDMLLLPDPETARIDPFRAAKTLNINFFVHDPFTLEPYSRDPRNIARKAENYLI | |
| Sbjct: | 1632 | FQSIHESDMLLLPDPETARIDPFRAAKTLNINFFVHDPFTLEPYSRDPRNIARKAENYLI | 1453 |
| Query: | 120 | STGIADTAYFGAEAEFYIFDSVSFDSRANGSFYEVDAISGWWNTGAATEADGSPNRGYKV | 179 |
| | | STGIADTAYFGAEAEFYIFDSVSFDSRANGSFYEVDAISGWWNTGAATEADGSPNRGYKV | |
| Sbjct: | 1452 | STGIADTAYFGAEAEFYIFDSVSFDSRANGSFYEVDAISGWWNTGAATEADGSPNRGYKV | 1273 |
| Query: | 180 | RHKGGYFPVAPNDQYVDLRDKMLTNLINSGFIVLEKGHHEGSGGQAEINYQFNSLLHAAD | 239 |
| | | RHKGGYFPVAPNDQYVDLRDKMLTNLINSGFILEKGHHEVGSGGQAEINYQFNSLLHAAD | |
| Sbjct: | 1272 | RHKGGYFPVAPNDQYVDLRDKMLTNLINSGFILEKGHHEVGSGGQAEINYQFNSLLHAAD | 1093 |
| Query: | 240 | DMQLYKYIIKNTAWQNGKTVTFMPKPLFGDNGSGMHCHQSLWKDGAPLMYDETGYAGLSD | 299 |
| | | DMQLYKYIIKNTAWQNGKTVTFMPKPLFGDNGSGMHCHQSLWKDGAPLMYDETGYAGLSD | |
| Sbjct: | 1092 | DMQLYKYIIKNTAWQNGKTVTFMPKPLFGDNGSGMHCHQSLWKDGAPLMYDETGYAGLSD | 913 |
| Query: | 300 | TARHYIGGLLHHAPSLLAFTNPTVNSYKRLVPGY-APINLVYSQRNRSACVRIPITGSNP | 358 |
| | | TARHYIGGLLHHAPSLLAFTNPTVNSYKRLVPGY APINLVYSQRNRSACVRIPITGSNP | |
| Sbjct: | 912 | TARHYIGGLLHHAPSLLAFTNPTVNSYKRLVPGYEAPINLVYSQRNRSACVRIPITGSNP | 733 |
| Query: | 359 | KAKRLEFRSPDSSGNPYLAFSAMLMAGLDGIKNKIEPQAPVDKDLYELPPEEAASIPQTP | 418 |
| | | KAKRLEFRSPDSSGNPYLAFSAMLMAGLDGIKNKIEPQAPVDKDLYELPPEEAASIPQTP | |
| Sbjct: | 732 | KAKRLEFRSPDSSGNPYLAFSAMLMAGLDGIKNKIEPQAPVDKDLYELPPEEAASIPQTP | 553 |
| Query: | 419 | TQLSDVIDRLEADHEYLTEGGVFTNDLIETWISFKRENEIEPVNIRPHPYEFALYYDV | 476 (SEQ ID NO: 19) |
| | | TQLSDVIDRLEADHEYLTEGGVFTNDLIETWISFKRENEIEPVNIRPHPYEFALYYDV | |
| Sbjct: | 552 | TQLSDVIDRLEADHEYLTEGGVFTNDLIETWISFKRENEIEPVNIRPHPYEFALYYDV | 379 (SEQ ID NO: 20) |

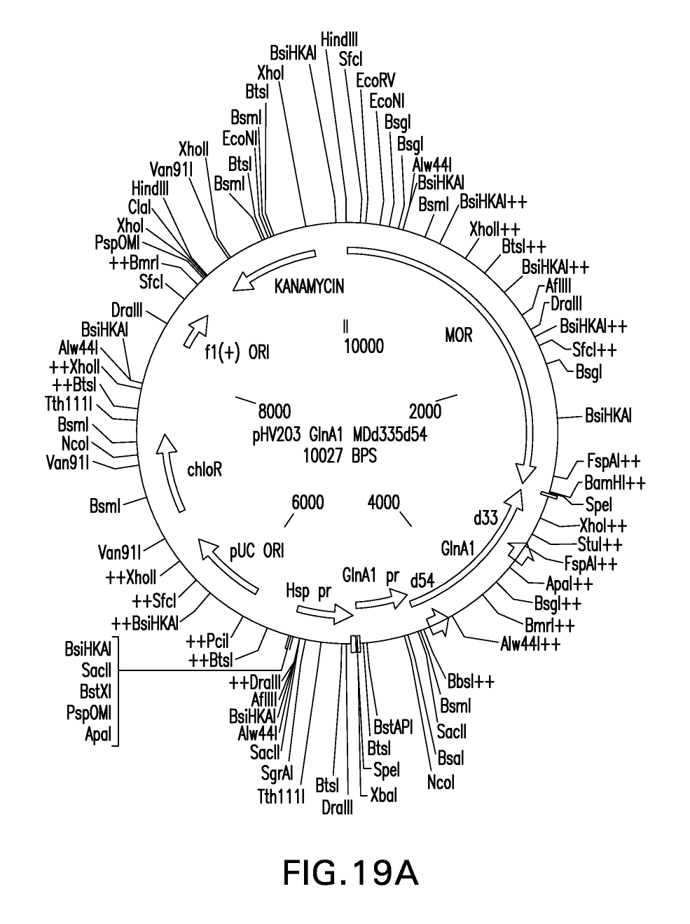

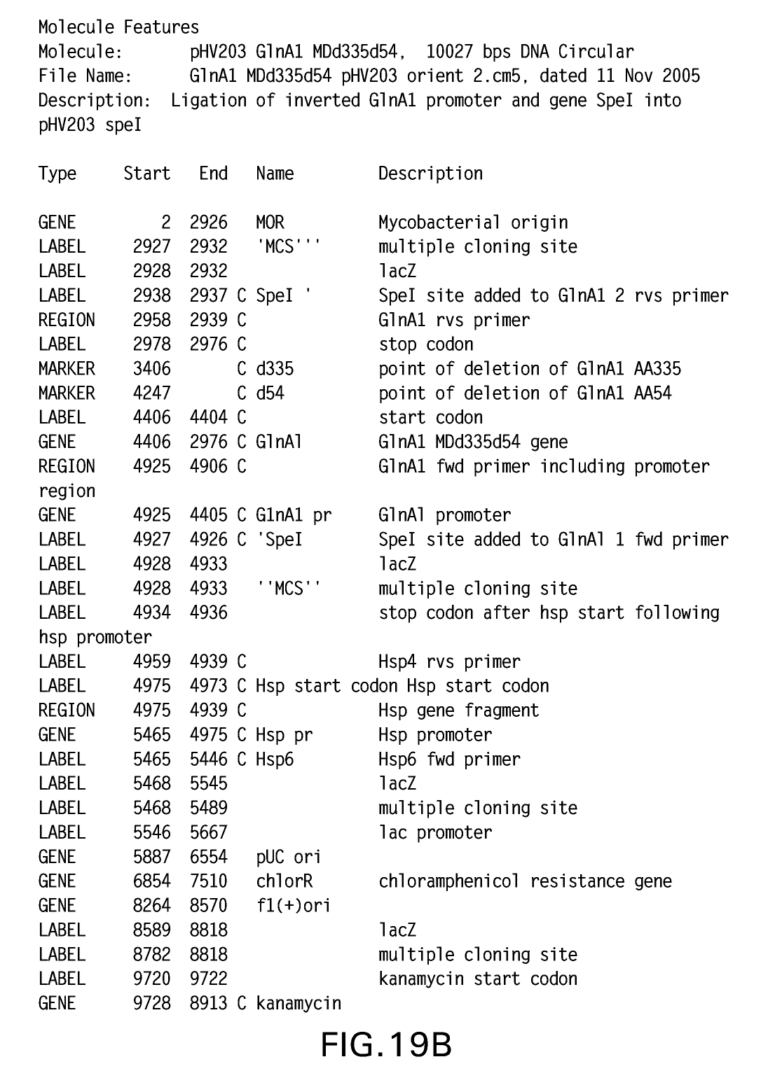

Next, the mutant glnA1 allele including its own promoter region was ligated into a speI site in pHV203 to yield pHV203-mut glnA1 ΔD54ΔE335 and also into the chromosomal integration vector pMP399 and the plasmid vector pMP349 promoter to yield pMP399-mut glnA1 ΔD54ΔE335 (SEQ ID NO: 31) and pMP349-mut glnA1 ΔD54ΔE335 (SEQ ID NO: 26) (Table 1). The pHV203-mut glnA1 ΔD54ΔE335 (SEQ ID NO: 28) plasmid map is shown in FIG. 19. The pHV203-mut glnA1 ΔD54ΔE335 plasmid was electroporated into the 3D-BCG vaccines to yield 4D-BCG vaccines. The vector pMP399-mut glnA1 ΔD54ΔE335 used to express the mutant glnA1 in BCG to create GLAD-BCG (chromosome-expressed). It can also be added to 1^{st}, and 2^{nd}, and 3^{rd} generation mutants of pro-apoptotic BCG to render, respectively, 2^{nd}, 3^{rd}, and 4^{th} generation pro-apoptotic BCG vaccines. These vectors can be introduced into BCG as well as 1st, 2nd, and 3rd generation pro-apoptotic BCG vaccines to yield, respectively, 1st, 2nd, 3rd, and 4th generation vaccines.

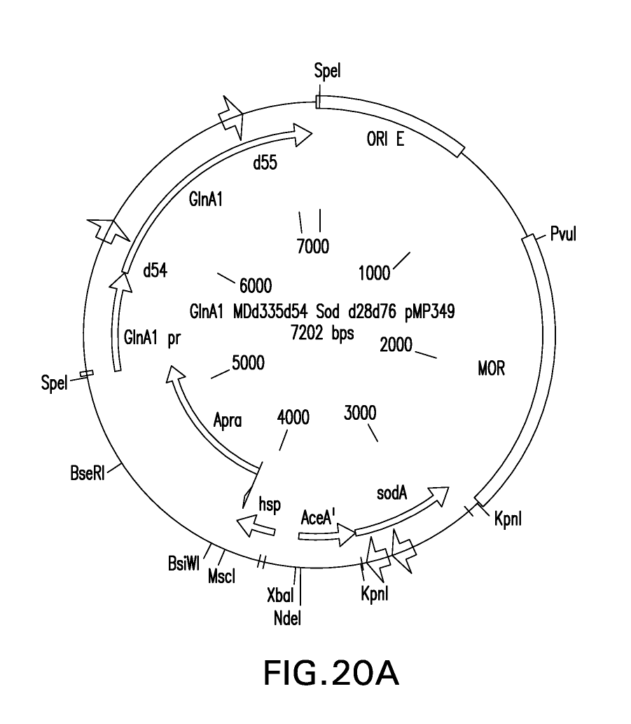

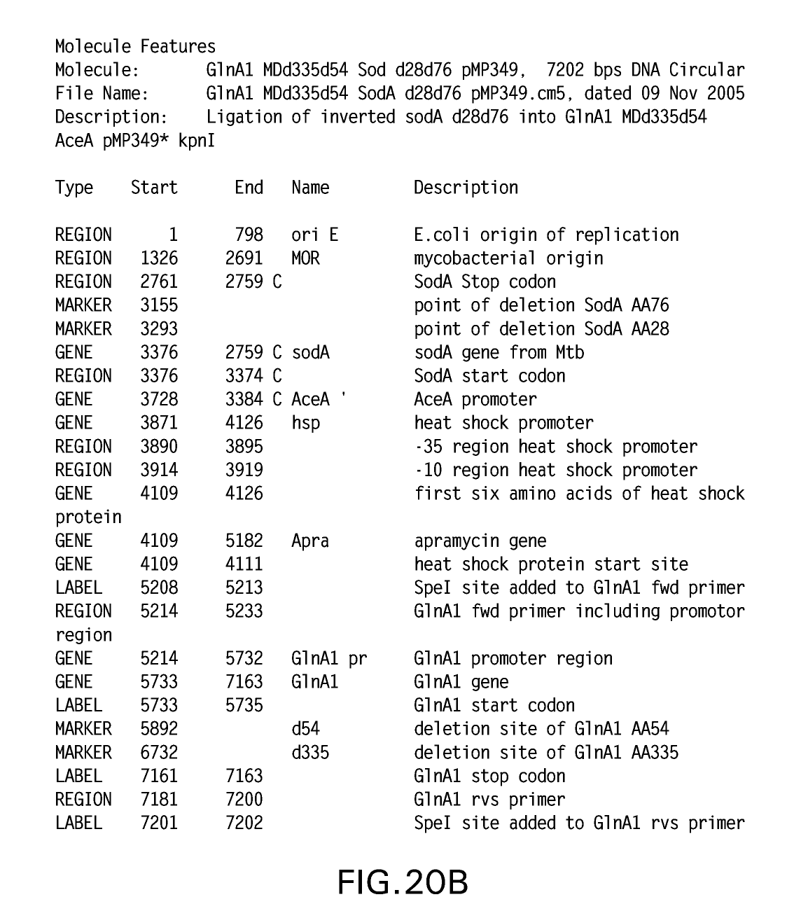

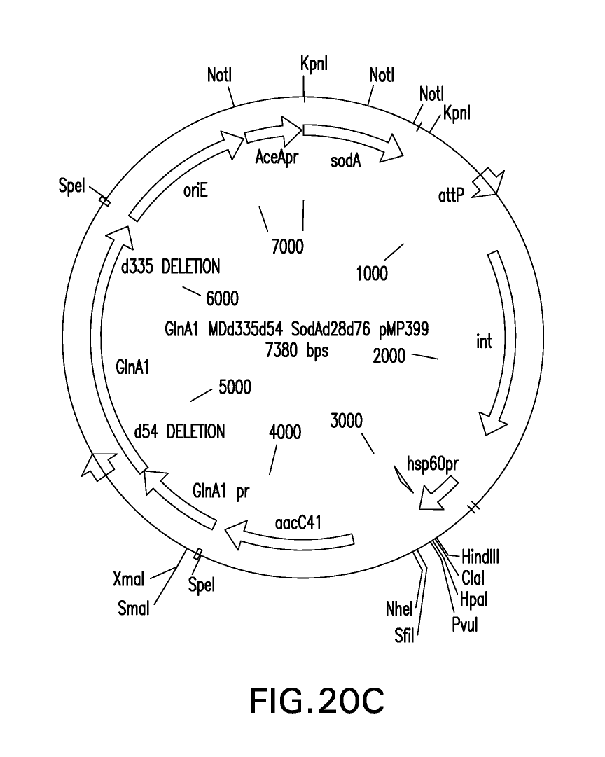

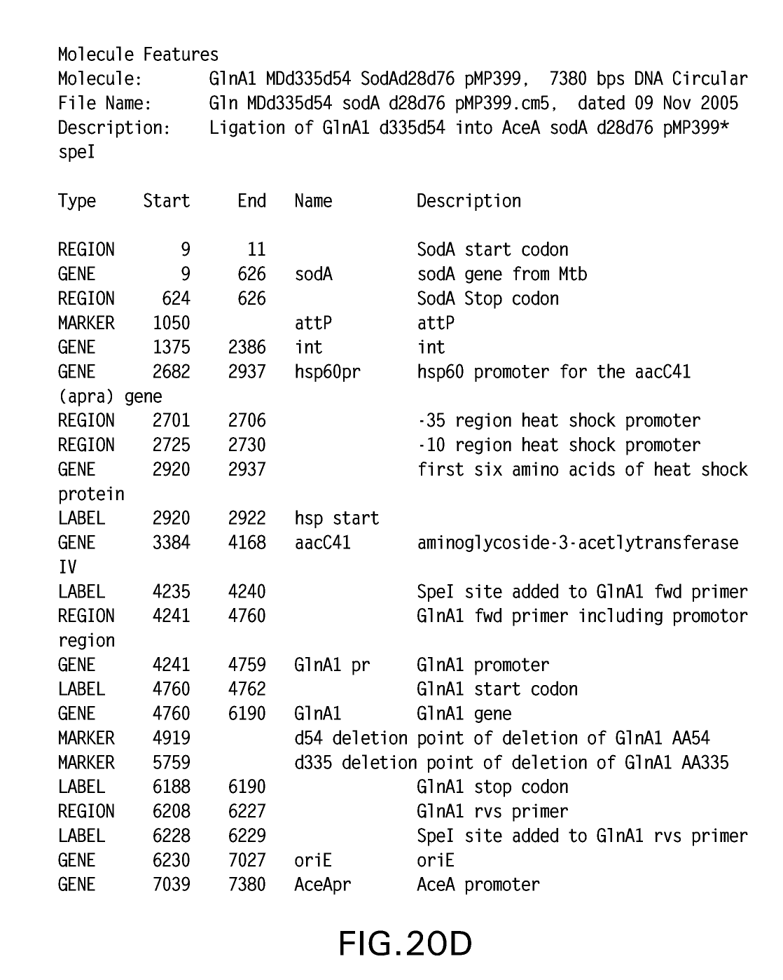

Additional plasmids and chromosomal-integration vectors were built that combined a mutant sodA allele and a mutant glnA1 allele on the same vector. These include pMP399-mut SodA ΔH28ΔH76 mut glnA1 ΔD54ΔE335 (FIG. 20), pMP399-mut SodA ΔE54 mut glnA1 ΔD54ΔE335, pMP349-mut SodA ΔH28ΔH76 mut glnA1 ΔD54ΔE335 (FIG. 20), and pMP349-mut SodA ΔE54 mut glnA1 ΔD54ΔE335 (Table 1). These vectors were introduced into BCG as well as 1st and 2nd generation pro-apoptotic BCG vaccines to yield, respectively, 2nd, 3rd, and 4th generation vaccines.

### Example 9

### Expression of an Exogenous Antigen by Pro-Apoptotic BCG

The pro-apoptotic BCG vaccines described above can be used to express exogenous antigens, including antigens from other infectious agents and cancer antigens.

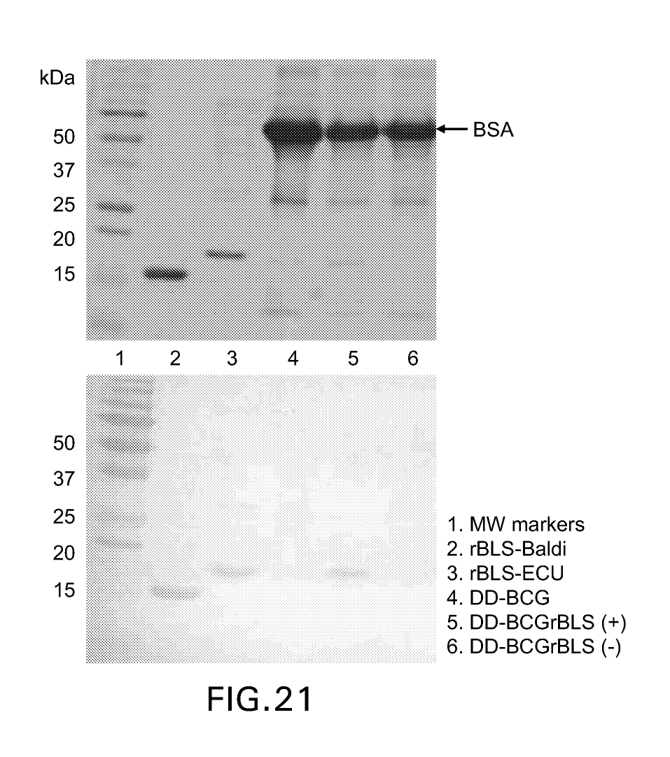

DD-BCGrBLS was constructed in which recombinant Brucella lumazine synthase, an immunodominant T-cell antigen of Brucella abortus, is expressed by DD-BCG. The bls gene was ligated behind an aceA(icl) promoter in pMP349 to produce pMP349-rBLS (SEQ ID NO: 38) (Table 1). This plasmid was electroporated into DD-BCG to yield DD-BCGrBLS. The expression of rBLS by DD-BCGrBLS is shown in FIG. 21. It can be added to BCG or to 1^{st}, 2^{nd}, 3^{rd} or 4^{th} generation pro-apoptotic BCG vaccines that enhance antigen presentation via apoptosis-associated cross priming pathways.

These results demonstrate that foreign antigens can be expressed in pro-apoptotic BCG. This capability allows the construction of a new generation of vaccines that induce strong T-cell responses by using pro-apoptotic intracellular bacteria as a vehicle for accessing apoptosis-associated cross priming pathways of antigen presentation. In this way, exogenous antigens can be delivered to dendritic cells to induce strong CD4 and CD8 T-cell responses. For example, the DD-BCGrBLS strain shown here or other pro-apoptotic intacellular bacterial vaccines expressing recombinant Brucella antigens can be used to immunize cattle or other mammalian hosts. This technology can be used to simultaneously protect cattle against bovine tuberculosis and brucellosis.

Due to differences in codon usage among different species, it is helpful to optimize codons in foreign genes for expression in mycobacteria. This can be done routinely by either using site-directed mutagenesis to alter the gene or by constructing synthetic genes that follow the codon usage preferences of mycobacteria. Such alterations are well-known to those skilled in the art.

### Example 10

### An Alternative to sigH Deletion Comprising Allelic Inactivation of Thioredoxin, Thioredoxin Reductase, and Glutaredoxin

The inactivation of sigH affects the production of multiple microbial factors, some of which are important targets for the host immune response. However the current data indicate that the low levels of sigH-regulated proteins expressed by a sigH deletion mutant are sufficient to induce strong T-cell responses against these proteins. However, as an alternative to sigH inactivation for pro-apoptotic BCG vaccines used to induce protection against tuberculosis, there is an advantage to directly reducing the activity of key anti-apoptotic enzymes under the control of sigH to minimize effects upon the stress-associated proteome. Under circumstances where the pro-apoptotic BCG vaccine is used primarily to express exogenous antigens from other infectious agents or cancer antigens, the sigH deletion is preferred and provides a mechanism for reducing the production of multiple anti-apoptotic antioxidants.

Thioredoxin (trxC, also trx, MPT46) and thioredoxin reductase (trxB2, also trxr) are sigH-regulated genes that are a prominent part of the bacterial response to oxidative stress. They are located adjacent to each other on the M. tuberculosis/BCG chromosome (trxB2 at bases 4,404,728-4,402,735 and trxC at 4,402,732-4,403,082 in the H37Rv chromosome, per complete genome sequence at TubercuList web server). A phasmid-based vector (pYUB854-trx-trxr) (SEQ ID NO: 35) to knock out both trxB2 and trxC simultaneously has been constructed, and the sequence data are provided in Table 1. The map and features of this vector are shown in FIG. 22. pYUB854-trx-trxr can be electroporated into BCG to construct BCGΔtrxΔtrxr. It can also be used to modify 1^{st}, 2^{nd}, and 3^{rd} generation pro-apoptotic BCG vaccines, respectively, into 2^{nd}, 3^{rd}, and 4^{th} generation pro-apoptotic BCG vaccines.

An alternative strategy for constructing TRX-TRXR-BCG (BCGΔtrxCΔtrxB2) involves the use of suicide plasmid vectors as described and referenced above, the use of which are well-known among those skilled in the art. One potential advantage of the plasmid-based system is greater ease in achieving unmarked deletions in which the allele is replaced by an inactive mutant rather than interrupted with an antibiotic resistance determinant. The active sites of thioredoxin, thioredoxin reductase, and many other redox repair enzymes contain active cysteines that form a disulfide bridge when oxidized. The “thioredoxin active-site motif' is a sequence of C-X-X-C where C=cysteine and X=any amino acids. This signature makes it routine to identify the active site of redox-active enzymes. Then the gene can be mutagenized or synthesized to eliminate the active site.

The following amino acid sequences of thioredoxin and thioredoxin reductase show the CXXC motifs in bold, at residues 37-40 and 145-148, respectively:

M. tuberculosis H37Rv|Rv3914|TrxC: 116 aa—THIOREDOXIN TRXC (TRX) (MPT46)

(SEQ ID NO: 21)
MTDSEKSATIKVTDASFATDVLSSNKPVLVDFWATWCGPCKMVAPVLEEI
ATERATDLTVAKLDVDTNPETARNFQVVSIPTLILFKDGQPVKRIVGAKG
KAALLRELSD VVPNLN

M. tuberculosis H37Rv|Rv3913|TrxB2: 335 aa—PROBABLE THIOREDOXIN REDUCTASE TRXB2 (TRXR) (TR)

(SEQ ID NO: 22)
MTAPPVHDRAHHPVRDVIVIGSGPAGYTAALYAARAQLAPLVFEGTSFGG
ALMTTTDVENYPGFRNGITGPELMDEMREQALRFGADLRMEDVESVSLHG
PLKSVVTADGQTHRARAVILAMGAAARYLQVPGEQELLGRGVS SCATCD
DGFFFRDQDIAVIGGGDSAMEEATFLTRFARSVTLVHRRDEFRASKIMLD
RARNNDKIRFLTNHTVVAVDGDTTVTGLRVRDTNTGAETTLPVT GVFVA
IGHEPRS GLVREAIDVDPDGYVLVQGRTTSTSLPGVFAAGDLVDRTYRQ
AVTAAGSGCAAAIDAERWLAEHAATGEADSTDALIGAQR

Using PCR-based gene mutagenesis techniques involving overlapping primers, genes encoding inactive mutants were constructed. The trxC allele encodes an inactive thioredoxin mutant that lacks the “WCGPCK” active-site and the trxB2 allele encodes an inactive thioredoxin reductase sequence that lacks the “SCATCD” active-site. These mutant alleles were incorporated into the p2NIL-pGOAL19 allelic inactivation vector system described by Parish and Stoker for introducing “unmarked” (i.e., the final construct lacks antibiotic resistance genes) to produce p2NIL/GOAL19-mut trxC-mut trxB2 (SEQ ID NO: 37) (FIG. 23 and Table 1). The vector for inactivating the active sites of thioredoxin (trxC, also trx) and thioredoxin reductase (trxB2, also trxr) without leaving residual antibiotic resistance. It can be electroporated into BCG to construct BCGΔtrxΔtrxr. It can also be used to modify 1^{st}, 2^{nd}, and 3^{rd} generation pro-apoptotic BCG vaccines, respectively, into 2^{nd}, 3^{rd}, and 4^{th} generation pro-apoptotic BCG vaccines.

This strategy can also be applied to other sigH-regulated genes. For example, RV2466c is sigH-regulated, is a glutaredoxin homolog, and possesses a C-X-X-C motif:

M. tuberculosis H37Rv|Rv2466c|Rv2466c: 207 aa—

(SEQ ID NO: 23)
MLEKAPQKSVADFWFDPLCPWCWITSRWILEVAKVRDIEVNFHVMSLAIL
NENRDDLPEQYREGMARAWGPVRVAIAAEQAHGAKVLDPLYTAMGNRIHN
QGNHELDEVITQSLADAGLPAELAKAATSDAYDNALRKSHHAGMDAVGED
VGTPTIHVNGVAFFGPVLSKIPRGEEAGKLWDASVTFASYPHFFELKRTR
TEPPQFD

### Example 11

### Deletion of Sigma Factor E (sigE) to Further Reduce the Production of Anti-Apoptotic Microbial Enzymes by BCG

As noted above, other sigma factors regulate the production of microbial factors important for the response to stress stimuli. Sigma factor E (sigE) has been shown to have an effect upon the production of SodA and gln1. Thus, inactivation of sigE introduces a defect in the production of microbial anti-apoptotic enzymes analogous to other defects described above, and thus can be used alone or combined with other mutations to make a pro-apoptotic BCG strain more potent.

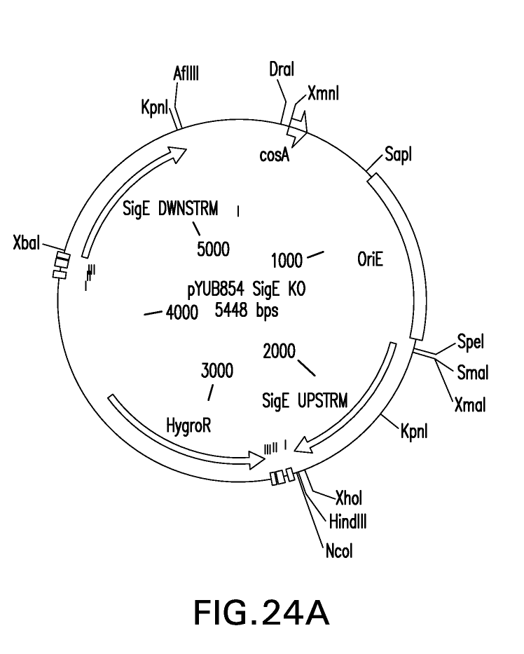

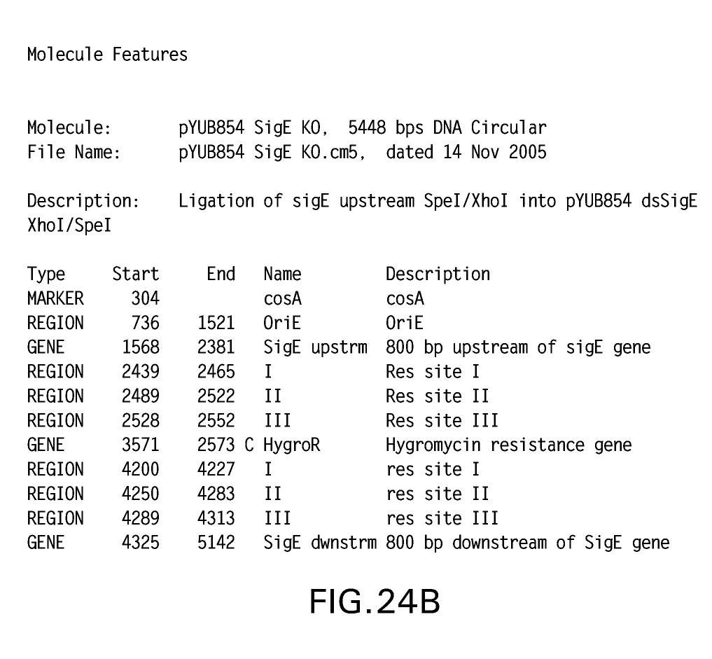

A phasmid-based vector (pYUB854-sigE) (SEQ ID NO: 36) to inactivate sigE has been constructed, and the sequence data are provided in Table 1. The map and features of this vector are shown in FIG. 24.

### Example 12

### Documentation of Reduced Glutamine Synthase Activity by 4D-BCG in vitro

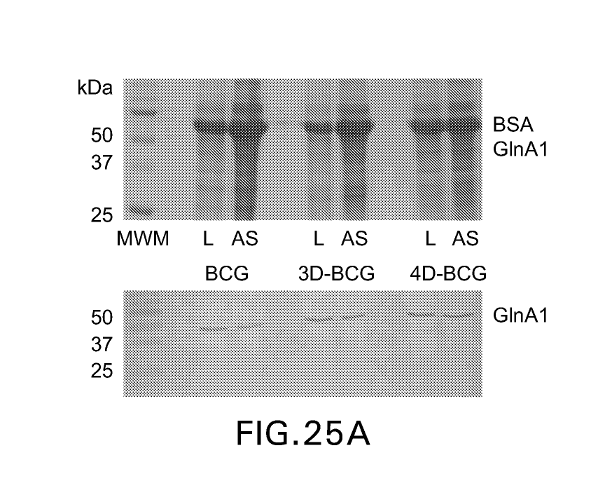

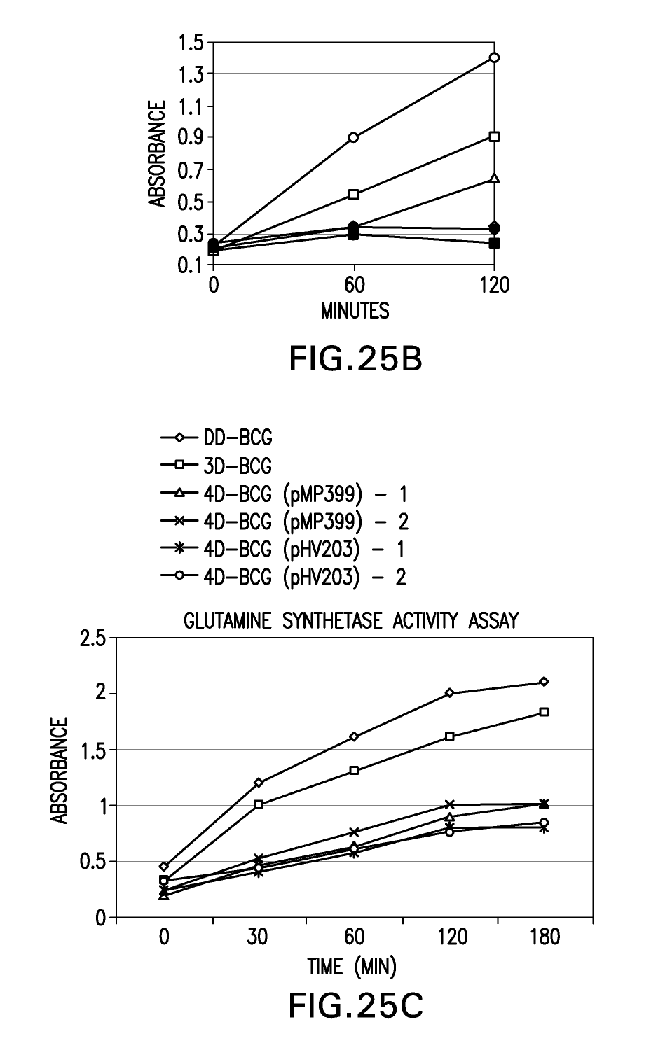

To determine the effect of expressing mutant ΔD55ΔE335 G1nA1 upon the glutamine synthetase activity of the whole bacterium, lysates of DD-BCG, 3D-BCG and two versions of 4D-BCG involving either plasmid or chromosomal expression of the mutant ΔD55ΔE335 GlnA1 were prepared and compared for glutamine synthetase activity. Activity assays were performed using the transfer reaction described by Woolfolk et al. by monitoring absorbance at 540 nm to detect the formation of gamma-glutamic acid hydroxamate. Results are shown in FIG. 25 and demonstrate that the dominant-negative strategy results in a 4- to 8-fold reduction in glutamine synthase activity.

### Example 13

### Splenocytes from Mice Vaccinated with DD-BCG, 3D-BCG, and 4D-BCG Exhibit Enhanced IL-2 Production Compared to Mice Vaccinated with the Parent BCG Strain

To evaluate immune responses to selected pro-apoptotic BCG (paBCG) vaccines and the parent BCG Tice vaccine strain, an IV vaccination model in C57Bl/6 mice was used, comprising administering approximately 5×10⁵ cfu of the vaccine strain as a single dose. Spleens are harvested and splenocytes are restimulated overnight on uninfected or BCG-infected bone marrow-derived macrophages (BMDMs) from these mice strains that have been stimulated with IFN-gamma to promote presentation of bacterial antigens. Thus, this is a very physiologic assay in which lymphocytes are harvested from vaccinated mice and then tested for their ability to make cytokines in response to an in vitro macrophage infection model that bears many similarities with in vivo infection. Intracellular cytokine staining (ICS) is performed with anti-CD3, anti-CD4, and anti-CD8 surface antibodies, and anti-IFN-gamma, anti-IL2 and anti-TNF-alpha intracellular antibodies. The specimens are then analyzed on a FACSaria sorter. BCG antigen-specific responses are determined by comparing IFN-γ, IL-2, and occasionally TNF-α production by splenocytes restimulated overnight on BCG-infected BMDMs versus cytokine production incubated overnight on uninfected BMDMs.

To determine immunologic responses, multiple experiments were performed comparing BCG, DD-BCG, 3D-BCG, and 4D-BCG (FIG. 26). After vaccination with BCG and DD-BCG sustained cytokine production was observed. About 0.7% of CD4 T-cells in the spleens of mice were able to produce IFN-γ in response to antigenic stimulation at day 70 post-vaccination. At 259 days post-vaccination, 0.30% and 0.27% of splenic CD4 cells still made IFN-γ in BCG and DD-BCG vaccinees, respectively. These results correlate with prolonged survival of both BCG and DD-BCG in the spleens of C57Bl/6 mice, a strain well-known for its “BCG-susceptibility” related to a mutant Nramp1 locus.

Differences in the production of specific cytokines were also noted. BCG-vaccinated mice exhibited a predominant IFN-γ response and the IL-2 production in BCG-vaccinated mice was not reliably above the natural variability in the assay (i.e., the range of IL-2 values observed in mice vaccinated with phosphate-buffered saline [sham-vaccinated controls] as indicated by the shaded area). When IL-2 production was observed in BCG-vaccinated mice, it was at low levels and detected around the time of the peak of the primary T-cell response at 4 weeks. In contrast, mice vaccinated with DD-BCG had fewer IFN-γ-producing CD4 cells relative to BCG-vaccinated mice but more IL-2-producing cells. The % of CD4+ T-cells producing IL-2 roughly correlated with the “generation” of paBCG vaccine under evaluation, and the induction of IL-2+ CD4+ T-cell responses was greater for 4D-BCG>3D-BCG>DD-BCG>BCG (FIG. 26A, lower panel). These results show that the pro-apoptotic modifications have an additive effect and when combined produce progressive enhancements in IL-2 production during primary vaccination.

The ratio of IFN-γ-producing to IL-2-producing CD4 cells in the same spleen typically averaged about 10:1 and 3:1 for recipients of BCG and the paBCG vaccines, respectively (FIG. 26B, in which the IL-2+ background values from uninfected BMDMs have been subtracted). This observation, combined with some other differences shown below, show that there is a qualitative enhancement in immune response induced by the paBCG vaccines compared to the immune response induced BCG.

The differences in cytokine production are best illustrated by comparing results around the peak of the primary T-cell response. FIG. 27 shows results from day 25 and day 31 post-vaccination in an experiment that compared BCG, DD-BCG, and 3D-BCG. In addition to the differences in IFN-γ production by CD4 T-cells (BCG>>DD-BCG>3D-BCG) and differences in IL-2 production by CD4+ T-cells (3D-BCG>>DD-BCG>BCG), the results also show increased IFN-γ production by CD8+ T-cells in the 3D-BCG-vaccinated mouse on day 25 (0.30%). Although the percentages of CD4 and CD8 IFN-γ-producing cells were identical, this mouse had a higher number of circulating CD8 cells, so in absolute terms the number of CD8+ IFN-γ+ cells was higher than the number of CD4 IFN-γ+ cells on day 25. Differences in values associated with DD-BCG versus 3D-BCG again indicate that each pro-apoptotic modification has an additive effect in enhancing the immunogenicity of BCG.

In summary, the pattern of T-cell effector cytokines induced by the paBCG vaccines during primary vaccination is different from the pattern of T-cell effector cytokines induced by BCG. As shown below in additional immunologic studies performed in the context of vaccination-challenge experiments, these differences during primary vaccination facilitate the development of memory responses that enable the vaccinated host to respond quickly to infection. The greater induction of IL-2 production by paBCG vaccine strains promotes T-cell growth, as the presence of IL-2 during the contraction phase of the primary T-cell response enhances the survival of antigen-specific T-cells, particularly memory T-cells.

### Example 14

### Enhanced Recall T-Cell Responses After Intratracheal Challenge of Mice Previously Vaccinated with 3D-BCG Compared to Mice Previously Vaccinated with BCG

The goal of vaccination is to generate a memory lymphocyte population in the immunized host that is directed against the infectious agent and can respond briskly to infection. To determine the kinetics and magnitude of recall T-cell responses, mice were subcutaneously vaccinated with 5×10⁵ cfu of BCG or 3D-BCG. Control mice were sham-vaccinated with phosphate-buffered saline (PBS). Thirty days following vaccination, mice were treated with antibiotics to eradicate any persisting vaccine bacilli. Although preliminary data indicate that the 3D-BCG and 4D-BCG vaccines are cleared as the adaptive immune response develops, BCG persists indefinitely in C57Bl/6 mice and in the spleen for at least five months after subQ vaccination. Thus, to avoid interference by the persistence of BCG, the vaccine strains were eliminated by treating all mice with isoniazid and rifampin in the drinking water starting at one month post-vaccination. This was found to be effective in reducing the number of BCG in the spleen below the lower limits of detection. After a month of treatment and an additional four weeks of rest, the mice receive an intratracheal challenge of 4×10⁷ cfu of BCG (all groups of mice, regardless of the initial vaccine strain). Baseline (day 0) numbers of cytokine+T-cells before challenge were low. Five days after challenge, the mice were euthanized and lungs were harvested to determine T-cell responses. The results are shown in FIG. 28 and show much stronger CD4+ T-cell responses in the mice vaccinated with 3D-BCG compared to the mice vaccinated with BCG. The 10-fold higher percent of IL-2+ CD4+ T-cells from mice vaccinated with 3D-BCG versus BCG recapitulates the greater IL-2 production seen during primary vaccination (FIGS. 26 and 27). Although the challenge dose used in this experiment is high/non-physiologic for TB infection, the design does allow for the ability to assess the rapidity of secondary T-cell responses under conditions of a relatively high antigen load. Thus, the results support the vector function of paBCG for delivering antigens of infectious agents that can rise to high titer very soon after inoculation (e.g., viral pathogens, malaria).

In summary, the secondary T-cell responses observed after challenge of mice vaccinated with 3D-BCG are stronger than secondary T-cell responses observed in mice vaccinated with BCG. The results show that paBCG is better than BCG in inducing a population of memory T-cells that can respond rapidly to challenge during a secondary (recall) response. Combined with greater attenuation and its ability to induce greater protection against tuberculosis than the current BCG vaccine, the immunologic studies highlight the use of paBCG as a platform technology for delivering exogenous antigens against other important infectious diseases and to target cancer.

### Example 15

### paBCG Enhances Recruitment and Activation of Neutrophils and NK Cells

The invention provides a highly effective vaccine against tuberculosis due to its ability to induce strong antigen-specific adaptive T cell responses that can be recalled during subsequent challenge. The development of strong adaptive immune requires an effective early host response that includes the recruitment and activation of key cells of the innate immune response to kill bacteria and present their antigens. To evaluate the innate immune responses to BCG and paBCG (3dBCG), C57Bl/6 mice were inoculated intravenously with 1.5×10⁷ CFU and performed gene microarrays 72 hours later upon splenic tissue (six mice per vaccination arm, and also six control mice vaccinated with phosphate-buffered saline).

The table below shows selected genes for which expression in the spleen was shown to differ in 3dBCG-vaccinated mice compared to BCG-vaccinated mice, as determined by using Affymetrix. In general, the results with different primer sets were highly consistent except in circumstances of very low gene expression (as assessed by the particular primer), and the relationship between gene expression in the vaccination arms are displayed as ratios and chip-to-chip comparisons. Genes are grouped together on the basis of function and several themes are noted. Briefly, these themes are:
1. Greater expression of cytokines and interleukins associated with memory immunity including IL-12p40, IL-15, and IL-18 in mice vaccinated with 3dBCG than in mice vaccinated with BCG.
2. Greater expression of genes associated with activation of macrophages and dendritic cells and with antigen presentation including CIITA, MHC Class II, CD1d1, CD80, and CD28 in mice vaccinated with 3dBCG than in mice vaccinated with BCG.
3. Greater recruitment and/or activation of neutrophils (including cathepsin G, cathelicidin, myeloperoxidase, and lipocalin 2) and cytotoxic lymphocytes including NK cells (including perform, CCL5, NK1.1, SLAM family receptors, and killer cell lectin-like receptors of the Ly49 family) in mice vaccinated with 3dBCG than in mice vaccinated with BCG.
4. Reduced expression of transferrin receptors (TfR) in mice vaccinated with 3dBCG than in mice vaccinated with BCG.

Whereas (1) and (2) have obvious implications for vaccine efficacy, (3) is also exciting as the anti-tumor effects of BCG as immunotherapy against cancer derives from its ability to recruit and activate innate immune cells. Observation (4) was a surprise but has important implications as discussed below.

It is also effective as an immunotherapeutic composition, and vaccine adjuvant that owes its improved results to the presence of a greater initial innate host response (i. e., greater rapid infiltration of inflammatory cells with apoptosis of host cells) when the production of anti-apoptotic enzymes by BCG is reduced. Furthermore, paBCG is better than BCG at recruiting and activating the types of innate immune cells (e.g., neutrophils and natural killer cells) that exhibit direct tumoricidal effects. The innate responses to paBCG includes the nonspecific activation and release of granules from NK and/or CD8+ T-cells in the first few days after administration/vaccination. It is also better at recruiting and activating macrophages and dendritic cells that can then present tumor antigens to induce strong adaptive T cell responses.

| | | | | | | | | |
|---|---|---|---|---|---|---|---|---|
| | | | | | Relationship of | | | |
| | | | | | BCG to Control | | | |
| | | | | | (I = increased; NC = | | | |
| | | | | | No Change; D = | | | |
| | | | | Ratio of | Decreased) by | | Ratio of | |
| Entrez | | | No. of | BCG to | Affymetrix | | 3dBCG to | |
| Gene | Gene | | primer | Control | Microarray chip-to- | Relationship of | BCG | Relationship of |
| ID | Symbol | Gene Title, Aliases | sets | Values | chip comparison | 3dBCG to Control | Values | 3dBCG to BCG |

Cytokines, Interleukins, Chemokines

| | | | | | | | | |
|---|---|---|---|---|---|---|---|---|
| 15978 | Ifng | interferon gamma | 1 | 3.3 | I | I | 1.2 | NC |
| 16145 | Igtp | interferon gamma | 1 | 4.5 | I | I | 0.8 | D |
| | | induced GTPase | | | | | | |
| 547223 | Il21 | interleukin 21 (similar | 1 | 36.7 | I | I | 1.3 | NC |
| | | to) | | | | | | |
| 16160 | Il12b | interleukin 12b, IL- | 2 | 1.0, 1.0 | NC, NC | I, NC | 1.3, 2.2 | MI, NC |
| | | 12p40 | | | | | | |
| 16189 | Il4 | interleukin 4 | 1 | 2.2 | NC | NC | 0.5 | NC |
| 16193 | Il6 | interleukin 6 | 1 | 0.1 | NC | NC | 18.3 | NC |
| 15979 | Ifngr1 | interferon gamma | 1 | 0.7 | D | NC | 1.4 | I |
| | | receptor 1, CD119 | | | | | | |
| 16168 | Il15 | interleukin 15 | 1 | 1.3 | NC | I | 1.4 | NC |
| 16196 | Il7 | interleukin 7 | 2 | 0.4-0.8 | D, NC | D, NC | 1.6, 2.5 | MI, NC |
| 16173 | Il18 | interleukin 18 | 1 | 1 | NC | I | 1.2 | I |
| 20308 | Ccl9 | CC chemokine ligand 9 | 2 | 0.8-1.0 | NC, NC | I, NC | 1.3, 1.6 | I, NC |
| 21926 | Tnf | tumor necrosis factor, | 1 | 1.7 | NC | NC | 1.1 | NC |
| | | tnf alpha | | | | | | |
| 20846 | Stat1 | signal transducer and | 4 | 2.7-3.2 | I, I, I, I | I, I, I, I | 0.9-1.0 | NC, NC, NC, NC |
| | | activator of | | | | | | |
| | | transcription 1 | | | | | | |
| 12703 | Socs1 | suppressor of | 2 | 2.7-2.8 | I, I | I, I | 0.8-1.1 | NC, NC |
| | | cytokine signaling 1 | | | | | | |

Neutrophils

| | | | | | | | | |
|---|---|---|---|---|---|---|---|---|
| 13035 | Ctsg | cathepsin G | 1 | 2.5 | I | I | 1.5 | I |
| 68891 | Cd177 | CD177 antigen | 1 | 1.6 | NC | I | 1.6 | I |
| | | cathelicidin | | | | | | |
| 12796 | Camp | antimicrobial peptide | 1 | 1.4 | I | I | 1.3 | I |
| 50701 | Ela2 | elastase 2, neutrophil | 1 | 2.2 | I | I | 1.2 | I |
| 17523 | Mpo | myeloperoxidase | 1 | 2.2 | I | I | 1.5 | I |
| 19152 | Prtn3 | proteinase 3 | 1 | 2.5 | I | I | 1.4 | I |
| 16819 | Lcn2 | lipocalin 2 | 1 | 1.1 | NC | I | 1.6 | I |
| 21946 | Pglyrp1 | peptidoglycan | 1 | 1 | NC | I | 1.6 | I |
| | | recognition protein 1 | | | | | | |
| 20201 | S100a8 | S100 calcium binding | 1 | 1.4 | I | I | 1.5 | I |
| | | protein A8 | | | | | | |
| | | (calgranulin A) | | | | | | |
| 20202 | S100a9 | S100 calcium binding | 1 | 1.6 | I | I | 1.3 | I |
| | | protein A9 | | | | | | |
| | | (calgranulin B) | | | | | | |

Microbicidal Capacity and Antigen Presentation

| | | | | | | | | |
|---|---|---|---|---|---|---|---|---|
| 17533 | Mrc1 | mannose receptor, C | 1 | 0.4 | D | D | 1.8 | I |
| | | type 1 | | | | | | |
| 11846 | Arg1 | arginase 1 | 1 | <0.1 | D | D | ~1 | NC |
| 170786 | Cd209a | CD209a antigen, DC- | 1 | 0.2 | D | D | 1.86 | NC |
| | | SIGN | | | | | | |
| 17076 | Ly75 | lymphocyte antigen | 1 | 1.2 | NC | I | 1.6 | NC |
| | | 75, CD205, DEC-205 | | | | | | |
| 17874 | Myd88 | myeloid differentiation | 1 | 1.6 | I | I | 1 | NC |
| | | primary response | | | | | | |
| | | gene 88 | | | | | | |
| 17087 | Ly96 | lymphocyte antigen | 1 | 0.8 | NC | MI | 1.5 | I |
| | | 96, MD-2 | | | | | | |
| 17872 | Myd116 | myeloid differentiation | 1 | 1.7 | I | D | 0.4 | D |
| | | primary response | | | | | | |
| | | gene 116 | | | | | | |
| 142980 | Tlr3 | toll-like receptor 3 | 2 | 0.9-1.3 | NC, NC | I, I | 1.3-2.3 | I, NC |
| 12265 | Ciita | class II transactivator | 2 | 0.7-0.8 | D, NC | NC, NC | 1.1-1.3 | NC, NC |
| 14961 | H2-Ab1 | histocompatibility 2, | 3 | 0.8-0.9 | D, D, NC | NC, NC, NC | 1.1, 1.1, 1.1 | I, I, NC |
| | | class II antigen A, | | | | | | |
| | | beta 1 | | | | | | |
| 14991 | H2-M3 | histocompatibility 2, M | 1 | 1.2 | NC | I | 1.2 | NC |
| | | region locus 3 | | | | | | |
| 12479 | Cd1d1 | CD1d1 antigen | 2 | 0.7-0.8 | D, D | NC, NC | 1.3-1.6 | NC, NC |
| 12519 | Cd80 | CD80 antigen | 2 | 0.8, 0.8 | NC, NC | NC, NC | 1.3-1.7 | NC, NC |
| 12487 | Cd28 | CD28 antigen | 2 | 0.8, 0.8 | D, D | NC, NC | 1.2, 1.3 | I, I |
| 11658 | Alcam | activated leukocyte | 5 | 0.5-0.7 | D, D, D, D, D | D, D, D, NC, NC | 0.9-1.6 | I, I, NC, NC, NC |
| | | cell adhesion | | | | | | |
| | | molecule, CD166 | | | | | | |
| 57765 | Tbx21 | T-box 21 | 1 | 1.1 | NC | MI | 1.3 | NC |

Iron Metabolism and Erythropoiesis

| | | | | | | | | |
|---|---|---|---|---|---|---|---|---|
| 22042 | Tfrc | transferrin receptor | 6 | 1.0-1.8 | I, I, I, NC, NC, NC | D, D, D, NC, NC, NC | 0.3-0.6 | D, D, D, D, D, D |
| 50765 | Trfr2 | transferrin receptor 2 | 2 | 1.7-1.9 | I, I | NC, NC | 0.5, 0.5 | D, D, D |
| 18719 | Pip5k1b | phosphatidylinositol- | 3 | 1.1-1.4 | NC, NC, NC | NC, NC, NC | 0.5-0.6 | D, D, NC |
| | | 4-phosphate 5-kinase, | | | | | | |
| | | type 1 beta | | | | | | |
| 14151 | Fech | ferrochelatase | 3 | 1.2-1.4 | I, I, NC | D, D, D | 0.4, 0.4 | D, D, D |
| 14319 | Fth1 | ferritin heavy chain 1 | 2 | 1.1, 1.1 | NC, NC | NC, NC | 0.9-1.0 | NC, NC |
| 14325 | Ftl1 | ferritin light chain 1/2 | 3 | 0.9, 0.9, 0.9 | NC, NC, NC | NC, NC, NC | 1.1, 1.1, 1.1 | NC, NC, NC |
| 84506 | Hamp1 | hepcidin antimicrobial | 3 | <0.1-0.1 | D, D, D | D, D, D | ~1 | NC, NC, NC |
| | | peptide 1/2 | | | | | | |
| 15216 | Hfe | hemochromatosis | 2 | 0.5, 0.7 | D, D | NC, NC | 1.1, 1.6 | I, I |
| 18173 | Slc11a1 | Nramp1-solute | 1 | 1.3 | I | I | 1.1 | NC |
| | | carrier family 11, | | | | | | |
| | | member 1 | | | | | | |
| 53945 | Slc40a1 | ferroportin-solute | 2 | 0.8, 0.8 | D, D | NC, NC | 1.2, 1.3 | I, I |
| | | carrier family 40, | | | | | | |
| | | member 1 | | | | | | |
| 11656 | Alas2 | aminolevulinic acid | 1 | 1.4 | I | D | 0.4 | D |
| | | synthase 2, erythroid | | | | | | |
| 69046 | Iscal | iron-sulfur cluster | 3 | 1.2-1.5 | I, I, I | D, D, D | 0.4-0.8 | D, D, D |
| | | assembly 1 homolog | | | | | | |
| | | (S. cerevisiae) | | | | | | |
| 64602 | Ireb2 | iron responsive | 1 | 0.8 | NC | NC | 1.1 | NC |
| | | element binding | | | | | | |
| | | protein 2 | | | | | | |
| 17002 | Ltf | lactotransferrin | 1 | 1.6 | NC | I | 1.1 | I |
| 13857 | Epor | erythropoietin | 1 | 1.4 | I | D | 0.5 | D |
| | | receptor | | | | | | |
| 12892 | Cpox | coproporphyrinogen | 3 | 1.1-1.6 | I, I, NC | D, D, D | 0.2-0.4 | D, D, D |
| | | oxidase | | | | | | |
| 22275 | Urod | uroporphyrinogen | 2 | 1.4-1.6 | I, I | D, D | 0.4-0.4 | D, D |
| | | decarboxylase | | | | | | |
| 22276 | Uros | uroporphyrinogen III | 2 | 1.5-1.6 | I, I | D, NC | 0.4-0.6 | D, NC |
| | | synthase | | | | | | |
| 16596 | Klf1 | Kruppel-like factor 1 | 1 | 2.1 | I | D | 0.3 | D |
| | | (erythroid) | | | | | | |
| 18022 | Nfe2 | nuclear factor, | 1 | 1.9 | I | NC | 0.4 | D |
| | | erythroid derived 2 | | | | | | |
| 630963 | Spna1 | spectrin alpha 1 | 2 | 1.8-1.9 | I, I | D, D | 0.2-0.3 | D, D |
| 13830 | Stom | stomatin | 6 | 0.9-1.3 | I, NC, NC, NC, NC | D, D, D, NC, NC | 0.4-0.9 | D, D, D, NC, NC |
| 66592 | Stoml2 | stomatin (Epb7.2)-like | 1 | 1.2 | I | NC | 0.9 | NC |
| | | 2 | | | | | | |
| 229277 | Stoml3 | stomatin (Epb7.2)-like | 2 | 1.8-2.1 | NC, NC | NC, NC | 0.2 | NC, NC |
| | | 3 | | | | | | |
| 11428 | Aco1 | aconitase 1 | 3 | 0.9-1.1 | NC, NC, NC | NC, NC, NC | 1.2 | NC, NC, NC |
| 269587 | Epb4.1 | erythrocyte protein | 4 | 0.9-1.7 | I, NC, NC, NC | D, D, D, NC | 0.5-0.8 | D, D, D, NC |
| | | band 4.1 | | | | | | |
| 13828 | Epb4.2 | erythrocyte protein | 2 | 1.8-1.9 | I, I | D, D | 0.2, 0.2 | D, D |
| | | band 4.2 | | | | | | |
| 14934 | Gypa | glycophorin A | 2 | 1.6-1.8 | I, I | D, D | 0.2, 0.2 | D, D |
| 71683 | Gypc | glycophorin C | 1 | 1.5 | I | NC | 0.6 | D |
| 232670 | Tspan33 | tetraspanin 33, | 4 | 1.3-1.6 | I, I, I, MI | D, D, MD, NC | 0.3-0.4 | D, D, D, D |
| | | penumbra | | | | | | |

Cytotoxic Lymphocytes including NK Cells

| | | | | | | | | |
|---|---|---|---|---|---|---|---|---|
| 14938 | Gzma | granzyme A | 1 | 2.3 | I | I | 1 | NC |
| 14939 | Gzmb | granzyme B | 1 | 4.1 | I | I | 1 | NC |
| 18646 | Prf1 | perforin 1 | 1 | 1.2 | NC | NC | 1.3 | NC |
| 20304 | Ccl5 | CC chemokine ligand 5 | 1 | 0.7 | D | NC | 1.4 | I |
| 13024 | Ctla2a | cytotoxic T | 2 | 0.9, 0.9 | ND, NC | NC, NC | 1.2-1.4 | I, I |
| | | lymphocyte- | | | | | | |
| | | associated protein 2 | | | | | | |
| | | alpha | | | | | | |
| 27218 | Slamf1 | SLAM, CD150 | 2 | 0.8-0.9 | D, NC | NC, NC | 1.5 | I, NC |
| 93970 | Klra18 | Ly49R-killer cell | 1 | 1 | NC | I | 2.3 | I |
| | | lectin-like receptor | | | | | | |
| | | A18 | | | | | | |
| 16639 | KIra8 | Ly49H-killer cell | 1 | 0.7 | D | NC | 2 | I |
| | | lectin-like receptor A8 | | | | | | |
| 18106 | Cd244 | Ly90-CD244 natural | 2 | 1.2-1.5 | I, NC | I, NC | 0.9-1.3 | I, NC |
| | | killer cell receptor 2B4 | | | | | | |
| 75345 | Slamf7 | CS1-SLAM family | 1 | 0.9 | NC | NC | 1.4 | I |
| | | member 7 | | | | | | |
| 12523 | Cd84 | CD84 antigen | 2 | 0.5-0.8 | NC, NC | NC, NC | 1.1-1.2 | NC, NC |
| 30925 | Slamf6 | Ly108, NTB-A-SLAM | 3 | 0.7-0.8 | D, NC, NC | NC, NC, NC | 0.8-1.2 | NC, NC, NC |
| | | family member 6 | | | | | | |
| 17059 | Klrb1c | Ly59, CD161, NK1.1- | 1 | 0.4 | D | NC | 2.1 | NC |
| | | killer cell lectin-like | | | | | | |
| | | receptor B1C | | | | | | |
| 27007 | Klrk1 | NKg2d - killer cell | 1 | 1.5 | I | I | 1.1 | NC |
| | | lectin-like receptor | | | | | | |
| | | subfamily K, member | | | | | | |
| | | 1 | | | | | | |
| 12525 | Cd8a | CD8 antigen, alpha | 5 | 0.8-1.1 | D, NC, NC, NC, NC | NC, NC, NC, NC, NC | 1.2-1.6 | I, NC, NC, NC, NC |
| | | chain | | | | | | |
| 20963 | Syk | spleen tyrosine kinase | 4 | 0.6-0.7 | D, D, D, D | D, D, D, MD | 1.0-1.2 | I, I, NC, NC |
| 22637 | Zap70 | zeta-chain (TCR) | 3 | 0.8-1.1 | NC, NC, NC | NC, NC, NC | 1.3-1.4 | NC, NC, NC |
| | | associated protein | | | | | | |
| | | kinase | | | | | | |
| 11891 | Rab27a | RAB27A, member | 3 | 0.9, 1.0, 1.0 | NC, NC, NC | I, NC, NC | 1.1-1.3 | NC, NC, NC |
| | | RAS oncogene family | | | | | | |
| 17967 | Ncam1 | CD56-neural cell | 2 | 0.2-0.4 | D, NC | NC, NC | 0.8-2.5 | I, NC |
| | | adhesion molecule 1 | | | | | | |
| 17748 | Mt1 | metallothionein 1 | 1 | 1.2 | NC | NC | 0.7 | D |
| 17750 | Mt2 | metallothionein 2 | 1 | 2.4 | I | I | 0.6 | D |

Macrophage-DC activation (also NK)-via G protein signalling pathways, prostaglandin, scavenger receptors, etc.

| | | | | | | | | |
|---|---|---|---|---|---|---|---|---|
| 64214 | Rgs18 | regulator of G-protein | 2 | 0.9, 0.9 | D, NC | NC, NC | 1.4, 1.5 | I, I |
| | | signaling 18 | | | | | | |
| 56470 | Rgs19 | regulator of G-protein | 2 | 0.8, 0.8 | D, NC | NC, NC | 1.3, 1.7 | I, I |
| | | signaling 19 | | | | | | |
| 19735 | Rgs2 | regulator of G-protein | 3 | 0.8, 0.8, 0.8 | D, D, D, NC | NC, NC, NC, NC | 1.3, 1.3, 1.3 | I, I, I, NC |
| | | signaling 2 | | | | | | |
| 18805 | Pld1 | phospholipase D1 | 3 | 0.5-0.7 | D, NC, NC | NC, NC, NC | 1.0-2.3 | I, NC, NC |
| 21672 | Prdx2 | peroxiredoxin 2 | 2 | 1.3, 1.5 | I, I | D, D | 0.5, 0.5 | D, D |
| 69810 | Clec4b1 | DCAR; C-type lectin | 2 | 0.8-0.9 | NC, NC | NC, NC | 1.5-1.6 | I, NC |
| | | domain family 4, | | | | | | |
| | | member b1 | | | | | | |
| 56620 | Clec4n | Dectin-2; C-type lectin | 2 | 1.0-1.1 | NC, NC | I, I | 1.3-1.5 | I, I |
| | | domain family 4, | | | | | | |
| | | member n | | | | | | |
| | | purinergic receptor | | | | | | |
| 70839 | P2ry12 | P2Y, G-protein | 1 | 0.6 | D | NC | 1.7 | I |
| | | coupled 12 | | | | | | |
| | | purinergic receptor | | | | | | |
| 74191 | P2ry13 | P2Y, G-protein | 1 | 0.8 | D | NC | 1.3 | I |
| | | coupled 13 | | | | | | |
| 67037 | Pmf1 | polyamine-modulated | 2 | 1.1-1.8 | NC, NC | NC, NC, NC | 0.6, 0.6 | D, NC |
| | | factor 1 | | | | | | |
| 18263 | Odc1 | ornithine | 3 | 1.3-1.9 | I, I, I | NC, NC, NC | 0.4-0.8 | D, D, NC |
| | | decarboxylase, | | | | | | |
| | | structural 1 | | | | | | |
| 228608 | Smox | spermine oxidase | 2 | 1.0-1.3 | I, NC | D, D | 0.5-0.6 | D, D |

B-cells, Antibodies

| | | | | | | | | |
|---|---|---|---|---|---|---|---|---|
| 14525 | Gcet2 | germinal center | 2 | 0.5, 0.5 | D, D | NC, NC | 1.8, 1.8 | I, I |
| | | expressed transcript 2 | | | | | | |
| 380794 | Ighg | Immunoglobulin | 2 | 0.8-1.0 | NC, NC | I, I | 1.7, 1.7 | |
| | | heavy chain (gamma | | | | | | I, I |
| | | polypeptide) | | | | | | |

Selected genes were analyzed further by RT-PCR (FIG. 29) and the results demonstrated greater expression of the anti-tumor cytokines IL-12 and IL-21 in recipients of the paBCG vaccine 3dBCG compared to recipients of the parent BCG Tice vaccine. In general, the results as determined by using microarrays with pooled specimens comprising the whole group of mice to be highly predictive of results determined on individual specimens by using RT-PCR.

Of note, the overloading of macrophages with iron as a consequence of increased expression of transferrin receptors in recipients of the parent BCG vaccine (FIG. 29) can induce a generalized immunosuppressive effect, as these cells are less capable of producing and responding to cytokines. This can in part explain the differences summarized in (1) and (2) above.

### Example 16

### Use of paBCG to Treat Bladder Cancer

### Intravesical use for Carcinoma In Situ of the Bladder.

Intravesical instillation of paBCG is indicated for the treatment of carcinoma-in-situ (CIS) of the bladder in the following circumstances:
1. The primary treatment of CIS of the bladder (after transurethral resection) either with or without associated papillary tumors.
2. The secondary treatment of CIS of the bladder in patients treated with other intravesical agents who have relapsed or failed to respond.
3. The primary or secondary treatment of CIS in patients who have contraindications to radical surgery.

### Intravesical use for TaTl Carcinoma of the Bladder.

Intravesical instillation of paBCG is indicated as an adjuvant treatment following transurethral resection of stage Ta or Tl papillary tumors of the bladder, which are at high risk of recurrence.

### Dosage Form, Route of Administration, and Recommended Dosage

PaBCG contains pro-apototic modifications of an attenuated live culture preparation of the Bacillus of Calmette and Guerin strain (BCG) of Mycobacterium bovis. Formulations of paBCG are described herein and in (U.S. Patent Application No. 20040109875).

The medium in which the paBCG organism is grown for the preparation of the freeze-dried cake is composed of the following: glycerin, asparagine, citric acid, potassium phosphate, magnesium sulfate, and iron ammonium citrate. The final preparation prior to freeze drying also contains lactose. No preservatives are added.

PaBCG is supplied as a freeze-dried powder in a box containing one vial. Each vial contains 1 to 8×10⁸ colony forming units, which is essentially equivalent to 50 mg (wet weight). The dose for the intravesical treatment of CIS and for prophylaxis of recurrent papillary tumors consists of one vial of paBCG suspended in 50 ml of preservative-free saline.

To prepare the BCG suspension, one ml of sterile-preservative free saline (0.9% sodium chloride USP) is drawn into a small syringe and then added to one vial of paBCG. After gentle mixing, the paBCG suspension is dispensed from the syringe into either another syringe which contains 49 ml of saline diluent or into a 50 ml plastic i.v. saline bag. The suspended paBCG can be used immediately after preparation and are be discarded after two hours.

PaBCG are be administered 7-14 days after bladder biopsy. Patients are not drink fluids for four hours before treatment and are empty their bladder prior to paBCG administration. The reconstituted paBCG is installed into the bladder by gravity flow using a catheter. paBCG are be retained in the bladder for two hours and then voided. While the paBCG is retained in the bladder, the patient are be repositioned from the left side to the right side and the back side to the abdomen every 15 minutes to maximize surface exposure to the agent.

A standard treatment schedule of paBCG consists of one intravesicular instillation per week for six weeks. The schedule can be repeated once if tumor remission has not been achieved and the clinical circumstances warrant. Thereafter, paBCG administration can be continued at monthly intervals for 6-12 months.

### Example 17

### Use of paBCG to Treat Melanoma

### Eligibility

Intralesional paBCG is usually considered for local or regional metastatic disease in lieu of more toxic systemic therapy where such a local approach can provide effective palliation and occasional cure. Such patients have a good performance status, ECOG≦3. Large lesions, >2 cm are unlikely to respond. This treatment is unlikely to be effective in the face of rapidly progressive disease with the appearance of many cutaneous lesions over a few days or weeks.

### Preparation

PaBCG is supplied as 1.5 mg of lyophilized powder and an additional diluent vial containing 1.5 ml saline. A separate supply of preservative-free saline is required for the lower doses.

Because of volume considerations, the lower doses, between 0.005 and 0.05 mg, require double dilution. Using the small diluent vial, the initial dilution is as instructed in the package insert to result in a concentration of 1 mg/ml. To make the double dilution, 0.9 ml of preservative-free saline is introduced into the now empty small diluent vial. To this is then added 0.1 ml from the initial dilution to result in a final double dilution concentration of 0.1 mg/ml. Doses of 0.005, 0.01, 0.02 and 0.04 mg can then be delivered in 0.05, 0.1, 0.2 and 0.4 ml, respectively. Higher doses, beginning at 0.1 mg, can be delivered using a single dilution only, by appropriate adjustment of diluent added to the lyophilized BCG. This is summarized in the following table:

BCCA Protocol Summary paBCG
1 of 3

| | Volume of | Volume to |
|---|---|---|
| Dose (mg) | Diluent (ml) | Deliver (ml) |
| 0.1 | 1.5 | 0.1 |
| 0.2 | 1.5 | 0.2 |
| 0.4 | 1.5 | 0.4 |
| 1.0 | 0.6 | 0.4 |
| 1.5 | 0.4 | 0.4 |

### Intralesional Administration:

PaBCG is delivered in a tuberculin syringe fitted with a 25 gauge needle. Injection is into the centre of a small lesion or at multiple sites for a larger lesion. Intracutaneous lesions do not retain fluid injected directly. In such cases it is better to insert the needle slightly distant from the lesion and advance it into the centre through the deep margin.

In consideration of occasional allergic reactions (see below), the first dose of 0.005 mg is given 30 min after administering intramuscular 50 mg diphenhydramine (BENADRYL®). The antihistamine is not routinely repeated with subsequent doses, but patients are remain under observation for 30 min following each injection.

Dose escalation is usually in the sequence detailed above for ‘lower’ and ‘higher’ doses. In each case this is an approximate two fold change between doses. Escalation of weekly injections continues until a dose is identified that causes a local inflammatory reaction or systemic symptoms. Further injections can be given at the same dose level every other week for two doses, then every month. Dose reductions may be indicated with significant increases in local or systemic reactivity. The total dose can be divided among several lesions where more than one is being treated.

Response may not be apparent for 4-6 weeks. After that, use of paBCG is reconsidered with clear evidence of progression of disease.

The same or similar approach can be taken with any other solid tumor under CT guidance, i.e., with current interventional radiology techniques, paBCG can be administered to other solid tumors, for example a lung cancer, a kidney cancer, or a liver metastasis.

When the primary tumor or a metastatic lesion has been previously undergone surgical resection, paBCG can be administered into or adjacent to the site of the removed tumor. This is done to facilitate the delivery of paBCG into the lymphatic vessels and lymph nodes along which tumor cells were likely to spread.

### Example 18

### Use of Modified BCG as an Adjuvant to Enhance the Activity of a Cancer Vaccine

The present protocol calls for the mixing of the cancer vaccine with paBCG organisms. For example, treated patients receive one intradermal vaccination per week for 2 weeks of about 10⁷ viable, irradiated autologous tumor cells and 10⁷ viable fresh-frozen paBCG organisms. For an example of this protocol, practiced with BCG to treat colon cancer.

### Example 19

### Use of Modified BCG as an Adjuvant to Enhance the Activity of a Killed Tumor Cell Vaccine

Autologous cancer cells are harvested from the patient, and treated (killed) by irradiation to prevent spread of metastatic disease upon re-introduction. The killed autologous tumor cells are admixed with paBCG, and administering by intradermal injection—e.g., the protocol cited in the context of Example 18. For an example of this protocol, practiced with BCG to treat colon cancer, see de Groot et al. Vaccine 23 (2005) 2379-2387.

A melanoma vaccine comprised of autologous melanoma cells or MVAX admixed with BCG is undergoing trials by AVAX and is described at: http://www.medicalnewstoday.com/articles/91442.php. The protocols described there are applicable to the present method using paBCG.

### Example 20

### Generation of Antisera to SodA to Prevent the Conversion from Latent TB Infection into Active Pulmonary TB, or to Reduce the Amount of Lung Damage in Active Pulmonary TB, or to Reduce Lung Fibrosis in Lung Infections Caused by Other Mycobacterium Species

The host response to Mycobacterium tuberculosis and other intracellular pathogens includes withholding iron, which is a co-factor for mycobacterial enzymes. Host transport mechanisms deplete the endocytic pathway of iron however pathogens express factors that compete for iron. Macrophages infected with non-pathogenic or pathogenic mycobacteria differ in iron content—for example, the phagosomes of MΦs infected with M. smegmatis are gradually depleted of iron, whereas iron accumulates within cells infected by M. tuberculosis or M. avium. Furthermore, pathogenic mycobacteria inhibit the fusion of lysosomes with phagosomes and reside in vacuoles that maintain a transferrin recycling pathway, thereby ensuring continued delivery of iron to the bacteria.

The ability of antioxidant-secreting mycobacteria to upregulate the expression of transferrin receptors (TfR) as shown above can play a central role in the iron-overloading of macrophages and thereby provide a mechanism by which M. tuberculosis (and BCG in certain hosts) induces lung pathology. SodA dismutates O₂⁻ to form H₂O₂ and these two oxidants have polar effects on the TfR mRNA binding/stabilizing activity of iron regulatory protein 1 (IRP1). Thus, the present results indicate a new model for the molecular mechanism by which lung damage occurs during pulmonary TB, i.e., by secreting SodA, M. tuberculosis promotes iron overload within MΦs and converts host-generated oxidants into toxic oxygen radicals that damage lung tissue. Stated another way, by using SodA to increase the expression of transferrin receptors (TfR) to a level that is inappropriate for the iron concentration, the bacterium forces the macrophage to acquire excess iron. Then, as the host responds to infection with TNF-α, IFN-γ, and other factors that promote the assembly of the NADPH oxidase and the production of reactive oxygen intermediates (ROIs), the ROIs are converted via Fenton and Haber-Weiss chemistry into tissue-damaging hydroxyl radicals (FIG. 30). In effect, the bacterium tricks the host into generating toxic oxidants that induce “bystander damage” to healthy tissue.

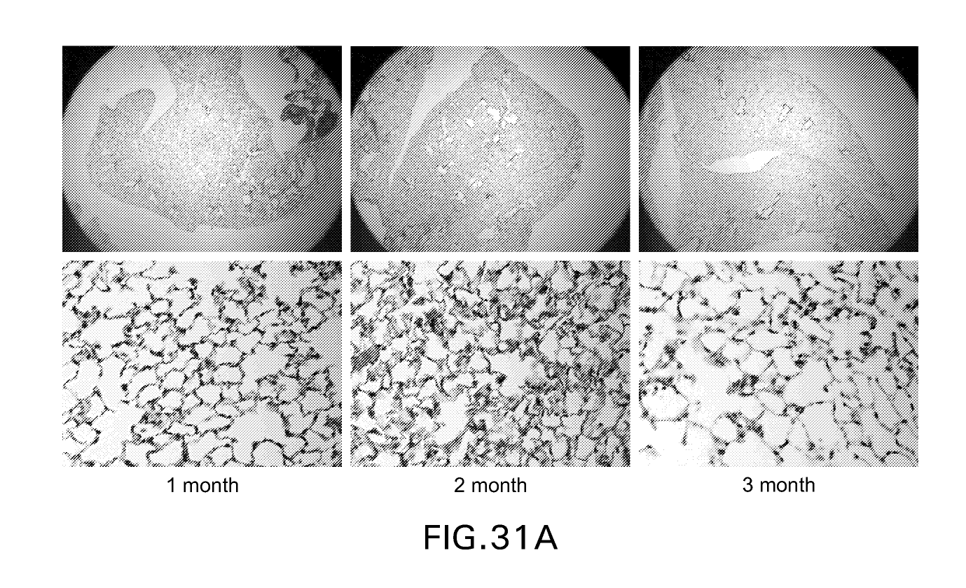

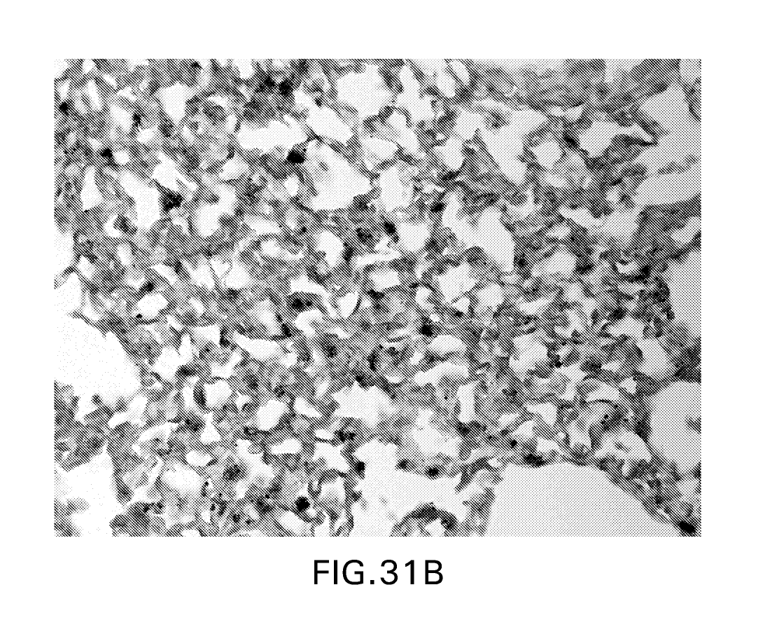

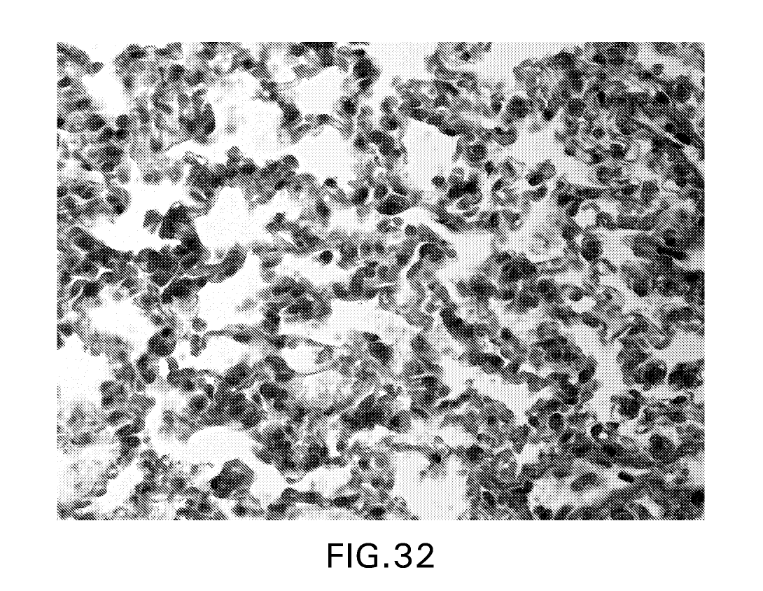

To determine that mycobacterial SodA promotes the uptake of iron by host macrophages and results in damage to lung tissue, M. tuberculosis SodA was expressed in a recombinant strain of the saprophytic Mycobacterium species M. vaccae, yielding MVrSodA. MVrSodA was then administered intratracheally to the lungs of C57Bl/6 mice. Following an early inflammatory response, hemosiderin-laden macrophages were prominent by two months post-infection (FIG. 31). By 16 weeks post-infection, a diffuse fibrosing response within the lung parenchyma was observed (FIG. 32).

Such results support the understanding that e superoxide dismutase-mediated iron uptake is probably crucial in the pathology of lung damage in response to infection by Mycobacterium species. This includes the transition from latent TB infection into active pulmonary TB and can also include lung fibrosis due to sarcoidosis, a disease of unclear etiology which has been associated with infection by a Mycobacterium species. Whereas latent TB infection (LTBI) is clinically silent, pulmonary TB causes damage to lung tissue. In effect, the transition from LTBI into active pulmonary TB begins with an increase in TfR expression that leads to iron uptake by macrophages and the generation of toxic oxygen radicals. Mycobacterial SodA induces the expression of TfR in infected cells and the maintenance of latency depends, in part, upon the host's ability to counter the effects of mycobacterial SodA and restrict TfR expression. The balance may be tipped in favor of the bacilli by conditions that limit the host's ability to restrict TfR expression. Then, the increased cellular iron enhances bacterial replication and induces a generalized immune suppressive effect, as iron-overloaded cells are less capable of producing and responding to cytokines including IFN-γ.

Thus, it is possible prevent the development of pulmonary TB in persons with latent TB infection by interfering with the activity of SodA. This can be done by generating antisera against mutant SodA purified directly from a Mycobacterium, or recombinant mutant SodA produced by another bacterial species or expression system, or a peptide derived from SodA. Ideally, the antisera have the property of neutralizing the enzymatic activity of SodA; however simply binding to SodA to facilitate elimination by the host can have a beneficial effect. Such antisera can be passively administered to a person with latent TB infection to reduce TfR expression and iron uptake in MΦ cultures and in vivo. Alternatively, a person with latent TB infection can be actively immunized with paBCG expressing dnSodA, recombinant SodA or mutant SodA, or a SodA peptide to induce the production of anti-SodA antibodies or cellular immune responses. Persons with active pulmonary TB or with infection by other Mycobacterium species that damage the lung can be similarly treated to reduce the amount of lung damage.

Live-attenuated vaccines are generally given to induce immunity against multiple antigens in naïve hosts. However there is also an urgent need for immune-based therapies in previously infected persons. A vaccine that specifically targets the transition from latent TB infection to active TB has negligible potential to instead cause aggravated disease (i.e., the Koch phenomenon). The need for immune therapy in LTBI has been made more urgent by the growing number of people infected with MDR- and XDR-TB strains that are difficult to treat with the usual antimicrobial agents because of pre-existing bacterial resistance.

TABLE 1
Bacterial strains, tools for genetic manipulations, and genetic constructs

| Strains and | | |
|---|---|---|
| genetic tools | | |
| Strains | Description | Reference or source |
| H37Rv | Virulent M. tuberculosis reference strain, | ATCC 25618 |
| | source of template chromosomal DNA for | |
| | gene mutations | |
| Erdman | Virulent M. tuberculosis reference strain, | ATCC 35801 |
| | commonly used as challenge strain in | |
| | experiments to evaluate vaccine efficacy | |
| AcrR-Erdman | Acriflavin-resistant mutant of Erdman, | Sheldon Morris, FDA |
| | also used as challenge strain for vaccine | [Repique, C. J. et al, |
| | efficacy | 2002] |
| TOP 10 | Host strain for cloning PCR products, | Invitrogen Corp., |
| | used in combination with pCR2.1-TOPO | Carlsbad, California |
| DH5α | E. coli host strain for genetic | [Hanahan, D., 1983] |
| | manipulation, construction of mutant | |
| | enzyme expression vectors | |
| BCG Tice | Bacillus Calmette-Guerin, substrain Tice | Organon Teknika |
| | | Corp., Durham, NC |
| SD-BCG-AS-SOD | SodA-diminished BCG containing either | [Edwards, K. M. et al, |
| | pHV203-AS-SOD or pLUC10-AS-SOD | 2001] and WO |
| | to practice antisense strategy | 02/062298 |
| C-BCG | Control BCG with either pHV203 or | [Edwards, K. M. et al, |
| | pLUC10 plasmid containing 151-bp of | 2001] and WO |
| | SodA but not in antisense orientation | 02/062298 |
| BCG (pLou1-mut | BCG with pLou1 chromosomal | Work related to the |
| SodA) | integration vector expressing mutant | teachings of WO |
| | SodA - BCG(pLou1-mut SodA) strains | 02/062298, mutant |
| | containing the following mutant SodA | SodA enzymes |
| | genes were constructed: H76K, ΔG134, | described in Table 11 |
| | H145K, H164K, ΔV184 | |

1^{st} generation pro-apoptotic BCG vaccines

| | | |
|---|---|---|
| SAD-BCGΔE54 | SodA-diminished BCG containing either | This invention |
| (aka SD-BCG | pMP349-mut SodA ΔE54 or pMP399- | |
| ΔE54) | mut SodA ΔE54 to practice dominant- | |
| | negative strategy | |
| SAD-BCG | SodA-diminished BCG containing either | This invention |
| ΔH28ΔH76 (aka | pMP349-mut SodA ΔH28ΔH76 or | |
| SD-BCG | pMP399-mut SodA ΔH28ΔH76 | |
| ΔH28ΔH76) | | |
| SIG-BCG (aka | BCG with allelic inactivation of sigH | This invention |
| BCGΔsigH) | | |
| SEC-BCG (aka | BCG with allelic inactivation of secA2 | Miriam Braunstein, |
| BCGΔsecA2) | | UNC, Chapel Hill |
| | | using methods |
| | | described in |
| | | [Braunstein, M. et al, |
| | | 2003; Braunstein, M. |
| | | et al, 2002] |
| GLAD-BCG | glnA1-diminished BCG containing either | This invention |
| | pMP349-mut glnA1 ΔD54ΔE335, | |
| | pHV203-mut glnA1 ΔD54ΔE335, or | |
| | pMP399-mut glnA1 ΔD54ΔE335 to | |
| | practice dominant-negative strategy | |

2^{nd} generation pro-apoptotic BCG vaccines

| | | |
|---|---|---|
| SAD-SIG-BCG | BCGΔsigH that is also sodA-diminished | This invention |
| ΔE54 (aka | by containing either pMP349-mut SodA | |
| BCGΔsigH ΔE54) | ΔE54 or pMP399-mut SodA ΔE54 | |
| SAD-SIG-BCG | BCGΔsigH that is also sodA-diminished | This invention |
| ΔH28ΔH76 (aka | by containing either pMP349-mut SodA | |
| BCGΔsigH | ΔH28ΔH76 or pMP399-mut SodA | |
| ΔH28ΔH76) | ΔH28ΔH76 | |
| SAD-SEC-BCG | BCGΔsecA2 that is also sodA-diminished | This invention |
| ΔE54 (aka | by containing either pMP349-mut SodA | |
| BCGΔsecA2 ΔE54) | ΔE54 or pMP399-mut SodA ΔE54 | |
| SAD-SEC-BCG | BCGΔsecA2 that is also sodA-diminished | This invention |
| ΔH28ΔH76 (aka | by containing either pMP349-mut SodA | |
| BCGΔsecA2 | ΔH28ΔH76 or pMP399-mut SodA | |
| ΔH28ΔH76) | ΔH28ΔH76 | |
| DD-BCG (aka | BCGΔsigHΔsecA2, also referred to as | This invention |
| BCGΔsigHΔsecA2) | “double-deletion” BCG | |
| GLAD-SIG-BCG | BCGΔsigH that is also glnA1-diminished | This invention |
| (aka BCGΔsigH | by containing either pMP349-mut glnA1 | |
| mut glnA1) | ΔD54ΔE335 or pMP399-mut glnA1 | |
| | ΔD54ΔE335 | |
| GLAD-SEC-BCG | BCGΔsecA2 that is also glnA1- | This invention |
| (aka BCGΔsecA2 | diminished by containing either pMP349- | |
| mut glnA1) | mut glnA1 ΔD54ΔE335 or pMP399-mut | |
| | glnA1 ΔD54ΔE335 | |
| GLAD-SAD-BCG | glnA1- and SodA-diminished BCG due to | This invention |
| ΔE54 | overexpression of mut glnA1 | |
| | ΔD54ΔE335 PLUS mut SodA ΔE54 | |
| GLAD-SAD-BCG | glnA1- and SodA-diminished BCG due to | This invention |
| ΔH28ΔH76 | overexpression of mut glnA1 | |
| | ΔD54ΔE335 PLUS mut SodA | |
| | ΔH28ΔH76 | |

3^{rd} generation pro-apoptotic BCG vaccines

| | | |
|---|---|---|
| 3D-BCG ΔE54 | DD-BCG that overexpresses mut SodA | This invention |
| | ΔE54 | |
| 3D-BCG | DD-BCG that overexpresses mut SodA | This invention |
| ΔH28ΔH76 | ΔH28ΔH76 | |
| GLAD-DD-BCG | DD-BCG that overexpresses mut glnA1 | This invention |
| | ΔD54ΔE335 | |
| GLAD-SAD-SIG- | BCGΔsigH that overexpresses mut glnA1 | This invention |
| BCG ΔE54 | ΔD54ΔE335 PLUS mut SodA ΔE54 | |
| GLAD-SAD-SIG- | BCGΔsigH that overexpresses mut glnA1 | This invention |
| BCG ΔH28ΔH76 | ΔD54ΔE335 PLUS mut SodA | |
| | ΔH28ΔH76 | |
| GLAD-SAD-SEC- | BCGΔsecA2 that overexpresses mut | This invention |
| BCG ΔE54 | glnA1 ΔD54ΔE335 PLUS mut SodAΔE54 | |
| GLAD-SAD-SEC- | BCGΔsecA2 that overexpresses mut | This invention |
| BCG ΔH28ΔH76 | glnA1 ΔD54ΔE335 PLUS mut SodA | |
| | ΔH28ΔH76 | |

4^{th} generation pro-apoptotic BCG vaccines

| | | |
|---|---|---|
| 4D-BCG ΔE54 | DD-BCG that overexpresses mut glnA1 | This invention |
| | ΔD54ΔE335 PLUS mut SodA ΔE54 | |
| 4D-BCG | DD-BCG that overexpresses mut glnA1 | This invention |
| ΔH28ΔH76 | ΔD54ΔE335 PLUS mut SodA | |
| | ΔH28ΔH76 | |

Pro-apoptotic BCG expressing exogenous antigen

| | | |
|---|---|---|
| DD-BCGrBLS | DD-BCG expressing recombinant | This invention |
| | Brucella lumazine synthase, from | |
| | Brucella abortus | |

Plasmids

| | | |
|---|---|---|
| pCR2.1-TOPO | Plasmid for cloning PCR products | Invitrogen Corp., |
| | | Carlsbad, California |
| pBC SK+ | E. coli phagemid vector | Stratagene, La Jolla, |
| | | CA |
| pMP349 | E. coli - mycobacterial shuttle plasmid | Martin Pavelka |
| | containing aacC41 gene encoding | [Consaul, S. A. et al, |
| | apramycin resistance | 2004] |
| pMP349-mut SodA | pMP349 with ΔE54 mutant SodA gene | SEQ ID NO: 24 |
| ΔE54 | cloned behind aceA (icl) promoter - mut | |
| | SodA also contains H28R substitution | |
| pMP349-mut SodA | pMP349 with ΔH28ΔH76 mutant SodA | SEQ ID NO: 25 |
| ΔH28ΔH76 | gene cloned behind aceA (icl) promoter - | |
| | mut SodA also contains G→S substitution | |
| | at C-terminus | |
| pMP349-mut | pMP349 with ΔD54ΔE335 mutant glnA1 | SEQ ID NO: 26 |
| glnA1 ΔD54ΔE335 | gene with its own promoter | |
| pMP349-mut SodA | pMP349 with ΔH28ΔH76 mutant SodA | SEQ ID NO: 27 |
| ΔH28ΔH76, mut | gene cloned behind aceA (icl) promoter | |
| glnA1 ΔD54ΔE335 | and ΔD54ΔE335 mutant glnA1 gene with | |
| | its own promoter. It can also be added to | |
| | 1^{st} and 2^{nd} generation mutants of pro- | |
| | apoptotic BCG to render, respectively, 3^{rd} | |
| | and 4^{th} generation pro-apoptotic BCG | |
| | vaccines. | |
| pHV203* | E. coli-mycobacterial shuttle plasmid with | [Edwards, K. M. et al, |
| | kanamycin resistance gene | 2001] and WO |
| | | 02/062298 |
| pHV203-AS-SOD | pHV203 containing a 151-bp fragment of | [Edwards, K. M. et al, |
| | sodA cloned in an antisense orientation | 2001] and WO |
| | behind promoter of 65 kDa heat-shock | 02/062298 |
| | protein | |
| pHV203-mut | pHV203 with ΔD54ΔE335 mutant glnA1 | SEQ ID NO: 28 |
| glnA1 ΔD54ΔE335 | gene with its own promoter | |
| pLUC10 | E. coli-mycobacterial shuttle plasmid | Robert Cooksey, |
| | containing firefly luciferase gene | CDC, Atlanta, |
| | | Georgia [Cooksey, R. |
| | | C. et al, 1993] |
| pLUC10-AS-SOD | pLUC10 containing a 151-bp fragment of | [Edwards, K. M. et al, |
| | sodA cloned in an antisense orientation | 2001] and WO |
| | behind promoter of 65 kDa heat-shock | 02/062298 |
| | protein | |
| pY6002 | Plasmid containing aph gene from Tn903, | Richard Young, MIT |
| | conferring resistance to kanamycin | [Aldovini, A. et al, |
| | | 1993] |
| pBAK14 | E. coli-mycobacterial shuttle plasmid | Douglas Young, |
| | containing the origin of replication from | Hammersmith |
| | the M. fortuitum plasmid pAL5000 | Hospital, London |
| | | [Zhang, Y. et al, 1991] |
| p16R1 | E. coli-mycobacterial shuttle plasmid for | Douglas Young, |
| | expressing SodA in mycobacteria, with | Hammersmith |
| | hygromycin resistance gene | Hospital, London |
| pNBV-1 | E. coli-mycobacterial shuttle plasmid with | [Howard, N. S. et al, |
| | hygromycin resistance gene | 1995] |

Chromosomal integration vectors

| | | |
|---|---|---|
| pMH94 | E. coli-mycobacterial attB integration | [Lee, M. H. et al, |
| | vector | 1991] |
| pLou1 | E. coli-mycobacterial attB integration | Jim Graham, |
| | vector | University of |
| | | Louisville |
| pLou1-mut SodA | pLou1 containing mutant SodA, pLou1 | Work related to the |
| | containing the following mutant SodA | teachings of WO |
| | genes were constructed: pLou1-H76K, | 02/062298 |
| | pLou1-ΔG134, pLou1-H145K, pLou1- | |
| | H164K, pLou1-ΔV184 | |
| pMP399 | E. coli-mycobacterial attB integration | Martin Pavelka |
| | vector containing aacC41 gene encoding | [Consaul, S. A. et al, |
| | apramycin resistance | 2004] |
| pMP399-mut SodA | pMP399 with ΔE54 mutant SodA gene | SEQ ID NO: 29 |
| ΔE54 | cloned behind aceA (icl) promoter - mut | |
| | SodA also contains H28R substitution | |
| pMP399-mut SodA | pMP399 with ΔH28ΔH76 mutant SodA | SEQ ID NO: 30 |
| ΔH28ΔH76 | gene cloned behind aceA (icl) promoter - | |
| | mut SodA also contains G→S substitution | |
| | at C-terminus | |
| pMP399-mut | pMP399 with ΔD54ΔE335 mutant glnA1 | SEQ ID NO: 31 |
| glnA1 ΔD54ΔE335 | gene with its own promoter | |
| pMP399-mut SodA | pMP399 with Δ54 mutant SodA gene | SEQ ID NO: 32 |
| ΔE54, mut glnA1 | cloned behind aceA (icl) promoter and | |
| ΔD54ΔE335 | ΔD54ΔE335 mutant glnA1 gene with its | |
| | own promoter. It can also be added to 1^{st} | |
| | and 2^{nd} generation mutants of pro- | |
| | apoptotic BCG to render, respectively, 3^{rd} | |
| | and 4^{th} generation pro-apoptotic BCG | |
| | vaccines. | |
| pMP399-mut SodA | pMP399 with ΔH28ΔH76 mutant SodA | SEQ ID NO: 33 |
| ΔH28ΔH76, mut | gene cloned behind aceA (icl) promoter | |
| glnA1 ΔD54ΔE335 | and ΔD54ΔE335 mutant glnA1 gene with | |
| | its own promoter. It used to | |
| | simultaneously express the ΔH28ΔH76 | |
| | mutant sodA and the ΔD54Δ | |
| | E335 mutant glnA1 in BCG to create | |
| | GLAD-SAD-BCG ΔH28ΔH76 | |
| | (chromosome-expressed). It can also be | |
| | added to 1^{st} and 2^{nd} generation mutants of | |
| | pro-apoptotic BCG to render, | |
| | respectively, 3^{rd} and 4^{th} generation pro- | |
| | apoptotic BCG vaccines. | |

Allelic inactivation tools for chromosomal genes

| | | |
|---|---|---|
| pYUB854, | phasmid chromosomal gene inactivation | William Jacobs, Jr., |
| pHAE87, | system for mycobacteria | Albert Einstein |
| pHAE159 | | College of Medicine |
| | | [Braunstein, M. et al, |
| | | 2002] |
| pYUB854-sigH | phasmid system vector for sigH | SEQ ID NO: 34 |
| | inactivation, used to construct | |
| | BCGΔsigH | |
| pYUB854-trx-trxr | phasmid system vector for inactivation | SEQ ID NO: 35 |
| | of thioredoxin and thioredoxin | |
| | reductase, used to construct | |
| | BCGΔtrxΔtrxr | |
| pYUB854-sigE | phasmid system vector for sigE | SEQ ID NO: 36 |
| | inactivation, used to construct | |
| | BCGΔsigH. The vector can also be used | |
| | to modify pro-apoptotic BCG vaccines | |
| | to make them more immunogenic. | |
| p1NIL, p2NIL, | suicide plasmid system for use in allelic | [Parish, T. et al, |
| pGOAL17, | replacement in mycobacteria | 2000] |
| pGOAL19 | | |
| p2NIL/GOAL19- | suicide plasmid for introducing | SEQ ID NO: 37 |
| mut trxC-mut | unmarked active-site mutations into trxC | |
| trxB2 | and trxB2 | |

Exogenous antigen expression vectors

| | | |
|---|---|---|
| pMP349-rBLS | pMP349 with recombinant Brucella | SEQ ID NO: 38 |
| | lumazine synthase behind aceA(icl) | |
| | promoter | |

*Note:
the terms pHV202 and pHV203 are used interchangeably. pHV203 was derived from pHV202 by repairing a mutation in the promoter region of the 65 kDa heat-shock protein used to drive expression of antisense DNA, and the inclusion of a larger upstream region of DNA to enhance stability.

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.
Albert M L, Sauter B, Bhardwaj N. Dendritic cells acquire antigen from apoptotic cells and induce class I-restricted CTLs. Nature 1998; 392:86-9.
Aldovini A, Husson R N, Young R A. The uraA locus and homologous recombination in Mycobacterium bovis BCG. J. Bacteriol. 1993; 175:7282-9.
Andersen P, Smedegaard B. CD4⁺ T-cell subsets that mediate immunological memory to Mycobacterium tuberculosis infection in mice. Infect. Immun. 2000; 68:621-9.
Balcewicz-Sablinska M K, Keane J, Komfeld H, Remold H G. Pathogenic Mycobacterium tuberculosis evades apoptosis of host macrophages by release of TNF-R2, resulting in inactivation of TNF-alpha. J. Immunol. 1998; 161:2636-41.
Bardarov S, Kriakov J, Carriere C et al. Conditionally replicating mycobacteriophages: a system for transposon delivery to Mycobacterium tuberculosis. Proc. Natl. Acad. Sci. U.S.A 1997; 94:10961-6.
Barnes P F. Immunotherapy for tuberculosis: wave of the future or tilting at windmills? Am. J. Respir. Crit Care Med. 2003; 168:142-3.
Behr M A, Small P M. Has BCG attenuated to impotence? Nature 1997; 389:133-4.
Berthet F X, Lagranderie M, Gounon P et al. Attenuation of virulence by disruption of the Mycobacterium tuberculosis erp gene. Science 1998; 282:759-62.
Beyer W F, Jr., Fridovich I. Assaying for superoxide dismutase activity: some large consequences of minor changes in conditions. Anal. Biochem. 1987; 161:559-66.
Blattman J N, Grayson J M, Wherry E J, Kaech S M, Smith K A, Ahmed R. Therapeutic use of IL-2 to enhance antiviral T-cell responses in vivo. Nat. Med. 2003; 9:540-7.
Brandau S, Suttmann H, Riemensberger J et al. Perforin-mediated lysis of tumor cells by Mycobacterium bovis Bacillus Calmette-Guerin-activated killer cells. Clin. Cancer Res. 2000; 6:3729-38.
Braunstein M, Bardarov S S, Jacobs W R, Jr. Genetic methods for deciphering virulence determinants of Mycobacterium tuberculosis. Methods Enzymol. 2002; 358:67-99.
Braunstein M, Espinosa B J, Chan J, Belisle J T, Jacobs W R, Jr. SecA2 functions in the secretion of superoxide dismutase A and in the virulence of Mycobacterium tuberculosis. Mol. Microbiol. 2003; 48:453-64.
Carlisle J, Evans W, Hajizadeh R et al. Multiple Mycobacterium antigens induce interferon-gamma production from sarcoidosis peripheral blood mononuclear cells. Clin. Exp. Immunol. 2007; 150:460-8.
Cho S, Mehra V, Thoma-Uszynski S et al. Antimicrobial activity of MHC class I-restricted CD8+ T cells in human tuberculosis. Proc. Natl. Acad. Sci. U.S.A 2000; 97:12210-5.
Coleman J, Green P J, Inouye M. The use of RNAs complementary to specific mRNAs to regulate the expression of individual bacterial genes. Cell 1984; 37:429-36.
Consaul S A, Pavelka M S, Jr. Use of a novel allele of the Escherichia coli aacC4 aminoglycoside resistance gene as a genetic marker in mycobacteria. FEMS Microbiol. Lett. 2004; 234:297-301.
Cooksey R C, Crawford J T, Jacobs W R, Jr., Shinnick T M. A rapid method for screening antimicrobial agents for activities against a strain of Mycobacterium tuberculosis expressing firefly luciferase. Antimicrob. Agents Chemother. 1993; 37:1348-52.
Cooper J B, McIntyre K, Badasso M O et al. X-ray structure analysis of the iron-dependent superoxide dismutase from Mycobacterium tuberculosis at 2.0 Angstroms resolution reveals novel dimer-dimer interactions. J. Mol. Biol. 1995; 246:531-44.
Dussurget O, Stewart G, Neyrolles O, Pescher P, Young D, Marchal G. Role of Mycobacterium tuberculosis copper-zinc superoxide dismutase. Infect. Immun. 2001; 69:529-33.
Eddine A N, Kaufmann S H. Improved protection by recombinant BCG. Microbes. Infect. 2005; 7:939-46.
Edwards K M, Cynamon M H, Voladri R K et al. Iron-cofactored superoxide dismutase inhibits host responses to Mycobacterium tuberculosis. Am. J. Respir. Crit Care Med. 2001; 164:2213-9.
Eisenberg D, Gill H S, Pfluegl G M, Rotstein S H. Structure-function relationships of glutamine synthetases. Biochim. Biophys. Acta 2000; 1477:122-45.
Fine, P. E. M., Carneiro, I. A. M., Milstien, J. B., and Clements, J. D. Issues relating to the use of BCG in immunization programs. A discussion document. Document WHO/V&B/99.23. 1999. Geneva, World Health Organization Department of Vaccines and Biologicals. Ref Type: Pamphlet
Gene 1995; 166:181-2.
Gill H S, Pfluegl G M, Eisenberg D. Preliminary crystallographic studies on glutamine synthetase from Mycobacterium tuberculosis. Acta Crystallogr. D. Biol. Crystallogr. 1999; 55 (Pt 4):865-8.
Govoni G, Vidal S, Gauthier S, Skamene E, Malo D, Gros P. The Bcg/Ity/Lsh locus: genetic transfer of resistance to infections in C57BL/6J mice transgenic for the Nramp1 Gly169 allele. Infect. Immun. 1996; 64:2923-9.
Graham J E, Clark-Curtiss J E. Identification of Mycobacterium tuberculosis RNAs synthesized in response to phagocytosis by human macrophages by selective capture of transcribed sequences (SCOTS). Proc. Natl. Acad. Sci. U.S.A 1999; 96:11554-9.
Grode L, Seiler P, Baumann S et al. Increased vaccine efficacy against tuberculosis of recombinant Mycobacterium bovis bacille Calmette-Guerin mutants that secrete listeriolysin. J. Clin. Invest 2005; 115:2472-9.
Hanahan D. Studies on transformation of Escherichia coli with plasmids. J. Mol. Biol. 1983; 166:557-80.
Harmel R P, Jr., Zbar B, Rapp H J. Suppression and regression of a transplanted tumor in the guinea pig colon mediated by Mycobacterium bovis, strain BCG. J. Natl. Cancer Inst. 1975; 54:515-7.
Hess J, Miko D, Catic A, Lehmensiek V, Russell D G, Kaufmann S H. Mycobacterium bovis Bacille Calmette-Guerin strains secreting listeriolysin of Listeria monocytogenes. Proc. Natl. Acad. Sci. U.S.A 1998; 95:5299-304.
Hill F W, Rutten V P, Hoyer M H et al. Local bacillus Calmette-Guerin therapy for bovine vulval papilloma and carcinoma. Cancer Immunol. Immunother. 1994; 39:49-52.
Hinds J, Mahenthiralingam E, Kempsell K E et al. Enhanced gene replacement in mycobacteria. Microbiology 1999; 145 (Pt 3):519-27.
Ho S N, Hunt H D, Horton R M, Pullen J K, Pease L R. Site-directed mutagenesis by overlap extension using the polymerase chain reaction. Gene 1989; 77:51-9.
Hondalus M K, Bardarov S, Russell R, Chan J, Jacobs W R, Jr., Bloom B R. Attenuation of and protection induced by a leucine auxotroph of Mycobacterium tuberculosis. Infect. Immun. 2000; 68:2888-98.
Horwitz M A, Harth G, Dillon B J, Maslesa-Galic' S. Recombinant bacillus Calmette-Guerin (BCG) vaccines expressing the Mycobacterium tuberculosis 30-kDa major secretory protein induce greater protective immunity against tuberculosis than conventional BCG vaccines in a highly susceptible animal model. Proc. Natl. Acad. Sci. U.S.A 2000; 97:13853-8.
Howard N S, Gomez J E, Ko C, Bishai W R. Color selection with a hygromycin-resistance-based Escherichia coli-mycobacterial shuttle vector.
Jackson M, Phalen S W, Lagranderie M et al. Persistence and protective efficacy of a Mycobacterium tuberculosis auxotroph vaccine. Infect. Immun. 1999; 67:2867-73.
Jacobs W R, Jr., Tuckman M, Bloom B R. Introduction of foreign DNA into mycobacteria using a shuttle phasmid. Nature 1987; 327:532-5.
Kaushal D, Schroeder B G, Tyagi S et al. Reduced immunopathology and mortality despite tissue persistence in a Mycobacterium tuberculosis mutant lacking alternative sigma factor, SigH. Proc. Natl. Acad. Sci. U.S.A 2002; 99:8330-5.
Keane J, Remold H G, Kornfeld H. Virulent Mycobacterium tuberculosis strains evade apoptosis of infected alveolar macrophages. J. Immunol. 2000; 164:2016-20.
Kelley J J, III, Caputo T M, Eaton S F, Laue T M, Bushweller J H. Comparison of backbone dynamics of reduced and oxidized Escherichia coli glutaredoxin-1 using 15N NMR relaxation measurements. Biochemistry (Mosc). 1997; 36:5029-44.
Kernodle, D. S., Cynamon, M. H., Hager C C, Destefano, M. S., Tham, K., Bochan, M. R., and Edwards, K. M. A tuberculosis vaccine prototype constructed by diminishing an anti-apoptotic microbial factor. Clinical Infectious Diseases 33, 1154. 2001 (Abstract of the 2001 meeting of the Infectious Diseases Society of America).
Kernodle, D., Shoen, C., VanHook, S., Crozier, I., DeStefano, M., Hager, C., Price, J., Tham, K-T., and Cyanamon, M. Reducing SodA production by Bacillus Calmette-Guerin enhances immunogenicity and protects C57Bl/6 mice against granulomatous lung disease following challenge with Mycobacterium tuberculosis. Abstract #2045, p. 80, in Tuberculosis: Integrating Host and Pathogen Biology, Keystone Symposia, Whistler, British Columbia, Canada, Apr. 2-7, 2005.
Kuroda K, Brown E J, Telle W B, Russell D G, Ratliff T L. Characterization of the internalization of bacillus Calmette-Guerin by human bladder tumor cells. J. Clin. Invest 1993; 91:69-76.
Lakey D L, Voladri R K, Edwards K M et al. Enhanced production of recombinant Mycobacterium tuberculosis antigens in Escherichia coli by replacement of low-usage codons. Infect. Immun. 2000; 68:233-8.
Lee M H, Pascopella L, Jacobs W R, Jr., Hatfull G F. Site-specific integration of mycobacteriophage L5: integration-proficient vectors for Mycobacterium smegmatis, Mycobacterium tuberculosis, and bacille Calmette-Guerin. Proc. Natl. Acad. Sci. U.S.A 1991; 88:3111-5.
Manganelli R, Fattorini L, Tan D et al. The extra cytoplasmic function sigma factor sigma(E) is essential for Mycobacterium tuberculosis virulence in mice. Infect. Immun. 2004a; 72:3038-41.
Manganelli R, Provvedi R, Rodrigue S, Beaucher J, Gaudreau L, Smith I. Sigma factors and global gene regulation in Mycobacterium tuberculosis. J. Bacteriol. 2004b; 186:895-902.
Manganelli R, Voskuil M I, Schoolnik G K, Dubnau E, Gomez M, Smith I. Role of the extracytoplasmic-function sigma factor sigma(H) in Mycobacterium tuberculosis global gene expression. Mol. Microbiol. 2002; 45:365-74.
Manganelli R, Voskuil M I, Schoolnik G K, Smith I. The Mycobacterium tuberculosis ECF sigma factor sigmaE: role in global gene expression and survival in macrophages. Mol. Microbiol. 2001; 41:423-37.
McCord J M, Fridovich I. Superoxide dismutase. An enzymic function for erythrocuprein (hemocuprein). J. Biol. Chem. 1969; 244:6049-55.
McKinney J D, Honer zu Bentrup K., Munoz-Elias E J et al. Persistence of Mycobacterium tuberculosis in macrophages and mice requires the glyoxylate shunt enzyme isocitrate lyase. Nature 2000; 406:735-8.
McMurray D N. Determinants of vaccine-induced resistance in animal models of pulmonary tuberculosis. Scand. J. Infect. Dis. 2001; 33:175-8.
Miller B H, Shinnick T M. Evaluation of Mycobacterium tuberculosis genes involved in resistance to killing by human macrophages. Infect. Immun. 2000; 68:387-90.
Mosolits S, Nilsson B, Mellstedt H. Towards therapeutic vaccines for colorectal carcinoma: a review of clinical trials. Expert. Rev. Vaccines. 2005; 4:329-50.
Mostowy S, Tsolaki A G, Small P M, Behr M A. The in vitro evolution of BCG vaccines. Vaccine 2003; 21:4270-4.
Murray P J, Aldovini A, Young R A. Manipulation and potentiation of antimycobacterial immunity using recombinant bacille Calmette-Guerin strains that secrete cytokines. Proc. Natl. Acad. Sci. U.S.A 1996; 93:934-9.
Olsen A W, Brandt L, Agger E M, van Pinxteren L A, Andersen P. The influence of remaining live BCG organisms in vaccinated mice on the maintenance of immunity to tuberculosis. Scand. J. Immunol. 2004; 60:273-7.
Otsuki Y, Li Z, Shibata M A. Apoptotic detection methods—from morphology to gene. Prog. Histochem. Cytochem. 2003; 38:275-339.
Parish T, Stoker N G. Use of a flexible cassette method to generate a double unmarked Mycobacterium tuberculosis tlyA plcABC mutant by gene replacement. Microbiology 2000; 146:1969-75.
Pavelka M S, Jr., Jacobs W R, Jr. Comparison of the construction of unmarked deletion mutations in Mycobacterium smegmatis, Mycobacterium bovis bacillus Calmette-Guerin, and Mycobacterium tuberculosis H37Rv by allelic exchange. J. Bacteriol. 1999; 181:4780-9.
Pelicic V, Jackson M, Reyrat J M, Jacobs W R, Jr., Gicquel B, Guilhot C. Efficient allelic exchange and transposon mutagenesis in Mycobacterium tuberculosis. Proc. Natl. Acad. Sci. U.S.A 1997; 94:10955-60.
Raman S, Song T, Puyang X, Bardarov S, Jacobs W R, Jr., Husson R N. The alternative sigma factor SigH regulates major components of oxidative and heat stress responses in Mycobacterium tuberculosis. J. Bacteriol. 2001; 183:6119-25.
Rehse P H, Kumei M, Tahirov T H. Compact reduced thioredoxin structure from the thermophilic bacteria Thermus thermophilus. Proteins 2005.
Repique C J, Li A, Collins F M, Morris S L. DNA immunization in a mouse model of latent tuberculosis: effect of DNA vaccination on reactivation of disease and on reinfection with a secondary challenge. Infect. Immun. 2002; 70:3318-23.
Rodriguez A, Regnault A, Kleijmeer M, Ricciardi-Castagnoli P, Amigorena S. Selective transport of internalized antigens to the cytosol for MHC class I presentation in dendritic cells. Nat. Cell Biol. 1999; 1:362-8.
Rutten V P, Klein W R, De Jong W A et al. Immunotherapy of bovine ocular squamous cell carcinoma by repeated intralesional injections of live bacillus Calmette-Guerin (BCG) or BCG cell walls. Cancer Immunol. Immunother. 1991; 34:186-90.
Sambandamurthy V K, Wang X, Chen B et al. A pantothenate auxotroph of Mycobacterium tuberculosis is highly attenuated and protects mice against tuberculosis. Nat. Med. 2002; 8:1171-4.
Schaible U E, Winau F, Sieling P A et al. Apoptosis facilitates antigen presentation to T lymphocytes through MHC-I and CD1 in tuberculosis. Nat. Med. 2003; 9:1039-
Seder R A, Hill A V. Vaccines against intracellular infections requiring cellular immunity. Nature 2000; 406:793-8.
Serbina N V, Flynn J L. Early emergence of CD8⁺ T cells primed for production of type 1 cytokines in the lungs of Mycobacterium tuberculosis-infected mice. Infect. Immun. 1999; 67:3980-8.
Serbina N V, Liu C C, Scanga C A, Flynn J L. CD8+ CTL from lungs of Mycobacterium tuberculosis-infected mice express perforin in vivo and lyse infected macrophages. J. Immunol. 2000; 165:353-63.
Silva C L, Bonato V L, Lima V M, Faccioli L H, Leao S C. Characterization of the memory/activated T cells that mediate the long-lived host response against tuberculosis after bacillus Calmette-Guerin or DNA vaccination. Immunology 1999; 97:573-81.
Smith D A, Parish T, Stoker N G, Bancroft G J. Characterization of auxotrophic mutants of Mycobacterium tuberculosis and their potential as vaccine candidates. Infect. Immun. 2001; 69:1142-50.
Stanford J, Stanford C, Grange J. Immunotherapy with Mycobacterium vaccae in the treatment of tuberculosis. Front Biosci. 2004; 9:1701-19.
Steensma D P, Timm M, Witzig T E. Flow cytometric methods for detection and quantification of apoptosis. Methods Mol. Med. 2003; 85:323-32.
Steerenberg P A, De Jong W H, Elgersma A et al. Tumor infiltrating leukocytes (tils) during progressive tumor growth and BCG-mediated tumor regression. Virchows Arch. B Cell Pathol. Incl. Mol. Pathol. 1990; 59:185-94.
Sturgill-Koszycki S, Schaible U E, Russell D G. Mycobacterium-containing phagosomes are accessible to early endosomes and reflect a transitional state in normal phagosome biogenesis. EMBO J. 1996; 15:6960-8.
Sureda A, Hebling U, Pons A, Mueller S. Extracellular H2O2 and not superoxide determines the compartment-specific activation of transferrin receptor by iron regulatory protein 1. Free Radic. Res. 2005; 39:817-24.
Suttmann H, Jacobsen M, Reiss K, Jocham D, Bohle A, Brandau S. Mechanisms of bacillus Calmette-Guerin mediated natural killer cell activation. J. Urol. 2004; 172:1490-5.
Suttmann H, Riemensberger J, Bentien G et al. Neutrophil granulocytes are required for effective bacillus calmette-guerin immunotherapy of bladder cancer and orchestrate local immune responses. Cancer Res. 2006; 66:8250-7.
Tan J K, Ho V C. Pooled analysis of the efficacy of bacille Calmette-Guerin (BCG) immunotherapy in malignant melanoma. J. Dermatol. Surg. Oncol. 1993; 19:985-90.
Trunz B B, Fine P, Dye C. Effect of BCG vaccination on childhood tuberculous meningitis and miliary tuberculosis worldwide: a meta-analysis and assessment of cost-effectiveness. Lancet 2006; 367:1173-80.
Tullius M V, Harth G, Horwitz M A. Glutamine synthetase GlnA1 is essential for growth of Mycobacterium tuberculosis in human THP-1 macrophages and guinea pigs. Infect. Immun. 2003; 71:3927-36.
Vanselow B A, Abetz I, Jackson A R. BCG emulsion immunotherapy of equine sarcoid. Equine Vet. J. 1988; 20:444-7.
Velikovsky C A, Cassataro J, Giambartolomei G H et al. A DNA vaccine encoding lumazine synthase from Brucella abortus induces protective immunity in BALB/c mice. Infect. Immun. 2002; 70:2507-11.
Wagner D, Maser J, Lai B et al. Elemental analysis of Mycobacterium avium-, Mycobacterium tuberculosis-, and Mycobacterium smegmatis-containing phagosomes indicates pathogen-induced microenvironments within the host cell's endosomal system. J. Immunol. 2005; 174:1491-500.
Wang P F, Arscott L D, Gilberger T W, Muller S, Williams C H, Jr. Thioredoxin reductase from Plasmodium falciparum: evidence for interaction between the C-terminal cysteine residues and the active site disulfide-dithiol. Biochemistry (Mosc). 1999; 38:3187-96.
Winau F, Hegasy G, Kaufmann S H, Schaible U E. No life without death-apoptosis as prerequisite for T cell activation. Apoptosis. 2005; 10:707-15.
Winau F, Kaufmann S H, Schaible U E. Apoptosis paves the detour path for CD8 T cell activation against intracellular bacteria. Cell Microbiol. 2004; 6:599-607.
Woolfolk C A, Shapiro B, Stadtman E R. Regulation of glutamine synthetase. I. Purification and properties of glutamine synthetase from Escherichia coli. Arch. Biochem. Biophys. 1966; 116:177-92.
Thar B, Canti G, Rapp H J, Bier J, Borsos T. Regression of established oral tumors after intralesional injection of living BCG or BCG cell walls. Cancer 1979; 43:1304-7.
Zhang Y, Lathigra R, Garbe T, Catty D, Young D. Genetic analysis of superoxide dismutase, the 23 kilodalton antigen of Mycobacterium tuberculosis. Mol. Microbiol. 1991; 5:381-91.
Zhong L, Amer E S, Holmgren A. Structure and mechanism of mammalian thioredoxin reductase: the active site is a redox-active selenolthiol/selenenylsulfide formed from the conserved cysteine-selenocysteine sequence. Proc. Natl. Acad. Sci. U.S.A 2000; 97:5854-9.

1. A method of modifying a bacterium to enhance the immunogenicity of the bacterium, comprising genetically altering the bacterium to express a dominant-negative mutant of an anti-apoptotic enzyme, whereby the bacterium has enhanced immunogenicity in a subject.

2. A modified bacterium made in accordance with the method of claim 1.

3. An immunogenic composition comprising the modified bacterium of claim 2.

4. The method of claim 1, wherein the bacterium is attenuated.

5. The methods of claim 1, wherein the bacterium is selected from the group consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, and other Mycobacterium species.

6. The methods of claim 1, wherein the dominant-negative mutant is a dominant-negative mutant of SodA in which a deletion, insertion, and/or substitution of nucleotides in the naturally occurring nucleic acid encodes a molecule that interferes with the SOD activity of the organism.

7. The methods of claim 1, wherein the dominant-negative mutant is a dominant negative mutant of glutamine synthase in which a deletion, insertion, and/or substitution of nucleotides in the naturally occurring nucleic acid encodes a molecule that interferes with the glutamine synthase activity of the organism.

8. (canceled)

9. The method of claim 1, comprising a further pro-apoptotic modification.

10. The method of claim 9, wherein the further pro-apoptotic modification comprises one or more modification selected from the group consisting of inactivation of SigH, inactivation of sigE, inactivation of SecA2, reduction of thioredoxin activity, reduction of thioredoxin reductase activity, reduction of glutaredoxin activity, reduction of thiol peroxidase activity, and reduction of the activity of the NAD(P)H quinone reductase Rv3303c.

11. The method of claim 1, wherein the dominant-negative mutant is a mutant SodA having deletions of histidine at position 28 and histidine at position 76.

12. The method of claim 1, wherein the dominant-negative mutant is a mutant SodA having a deletion of histidine at position 28 or a histidine at position 76.

13. The method of claim 1, wherein the dominant-negative mutant is a mutant SodA having a deletion of glutamic acid at position 54.

14. The method of claim 1, wherein the dominant-negative mutant is a mutant SodA having a deletion of glutamic acid at position 54 and the replacement of histidine with arginine at position 28.

15. The method of claim 1, wherein the bacterium comprises a dominant-negative mutant of SodA and inactivation of sigH.

16. The method of claim 1, wherein the bacterium comprises a dominant-negative mutant of SodA and inactivation of secA2.

17. The method of claim 1, wherein the bacterium comprises a dominant-negative mutant of SodA, inactivation of sigH and inactivation of secA2.

18. The method of claim 1, wherein the bacterium comprises a dominant-negative mutant of SodA, a dominant-negative mutant of glnA1, inactivation of sigH and inactivation of secA2.

19. The method of claim 1, wherein the bacterium comprises a dominant negative mutation of glnA1.

20. The method of claim 19, wherein the dominant-negative mutant of glutamine synthase comprises deletions of aspartic acid at amino acid 54 and glutamic acid at amino acid 335.

21. The method of claim 19, wherein the dominant-negative mutant of glutamine synthase comprises a deletion of aspartic acid at amino acid 54 or a glutamic acid at amino acid 335.

22. The method of claim 20, wherein the bacterium further comprises inactivation of secA2.

23. The method of claim 22, wherein the bacterium further comprises inactivation of SodA.

24. The method of claim 23, wherein the dominant-negative mutant of SodA is a mutant SodA having deletions of histidine at position 28 and histidine at position 76.

25. The method of claim 20, wherein the bacterium further comprises an activity reducing mutation of sigH and inactivation of secA2.

26. The method of claim 20, wherein the bacterium further comprises a dominant-negative mutant of SodA and inactivation of sigH.

27. The method of claim 26, wherein the dominant-negative mutant is a mutant SodA having a deletion of glutamic acid at position 54.

28. The method of claim 26, wherein the dominant-negative mutant is a mutant SodA having deletions of histidine at position 28 and histidine at position 76.

29. The method of claim 20, wherein the bacterium further comprises a dominant-negative mutant of SodA and inactivation of secA2.

30. The method of claim 29, wherein the dominant-negative mutant is a mutant SodA having a deletion of glutamic acid at position 54.

31. The method of claim 29, wherein the dominant-negative mutant is a mutant SodA having deletions of histidine at position 28 and histidine at position 76.

32. The method of claim 4, wherein the bacterium comprises inactivation of sigH.

33. The method of claim 4, wherein the bacterium comprises inactivation of sigH and inactivation of secA2.

34. The modified bacterium of claim 2, wherein the bacterium is attenuated.

35. The modified bacterium of 2, wherein the bacterium is selected from the group consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, and other Mycobacterium species.

36. The modified bacterium of claim 2, wherein the dominant-negative mutant is a dominant-negative mutant selected from the group consisting of
a) SodA in which a deletion, insertion, and/or substitution of nucleotides in the naturally occurring nucleic acid encodes a molecule that reduces the SOD activity of the organism; and
b) glutamine synthase in which a deletion, insertion, and/or substitution of nucleotides in the naturally occurring nucleic acid encodes a molecule that reduces the glutamine synthase activity of the organism.

37. The modified bacterium of claim 36, wherein the bacterium is BCG.

38. The modified bacterium of claim 37, comprising a further pro-apoptotic modification.

39. The modified bacterium claim 38, wherein the further pro-apoptotic modification comprises one or more modification selected from the group consisting of inactivation of SigH, inactivation of sigE, inactivation of SecA2, reduction of thioredoxin activity, reduction of thioredoxin reductase activity, reduction of glutaredoxin activity, reduction of thiol peroxidase activity, and reduction of the activity of the NAD(P)H quinone reductase Rv3303c.

40. The modified bacterium claim 37, wherein the dominant-negative mutant is a mutant SodA having deletions of histidine at position 28 and histidine at position 76.

41. The modified bacterium claim 37, wherein the dominant-negative mutant is a mutant SodA having a deletion of histidine at position 28 or a histidine at position 76.

42. The modified bacterium claim 37, wherein the dominant-negative mutant is a mutant SodA having a deletion of glutamic acid at position 54.

43. The modified bacterium claim 37, wherein the dominant-negative mutant is a mutant SodA having a deletion of glutamic acid at position 54 and the replacement of histidine with arginine at position 28.

44. The modified bacterium of claim 39, wherein the bacterium comprises a dominant-negative mutant of SodA and inactivation of sigH.

45. The modified bacterium of claim 39, wherein the bacterium comprises a dominant-negative mutant of SodA and inactivation of secA2.

46. The modified bacterium of claims 39, wherein the bacterium comprises a dominant-negative mutant of SodA, an inactivation of sigH and inactivation of secA2.

47. The modified bacterium of claim 39, wherein the bacterium comprises a dominant-negative mutant of SodA, a dominant-negative mutant of glnA1, inactivation of sigH and inactivation of secA2.

48. The modified bacterium of claim 39, wherein the bacterium comprises a dominant negative mutation of glnA1.

49. The modified bacterium of claim 48, wherein the dominant-negative mutant of glutamine synthase comprises deletions of aspartic acid at amino acid 54 and glutamic acid at amino acid 335.

50. The modified bacterium of claim 48, wherein the dominant-negative mutant of glutamine synthase comprises a deletion of aspartic acid at amino acid 54 or a glutamic acid at amino acid 335.

51. The modified bacterium of claim 49, wherein the bacterium further comprises inactivation of secA2.

52. The modified bacterium of claim 51, wherein the bacterium further comprises a dominant-negative mutant of SodA.

53. The modified bacterium of claim 52, wherein the dominant-negative mutant of SodA is a mutant SodA having deletions of histidine at position 28 and histidine at position 76.

54. The modified bacterium of claim 49, wherein the bacterium further comprises inactivation of sigH and inactivation of secA2.

55. The modified bacterium of claim 49, wherein the bacterium further comprises a dominant-negative mutant of SodA and inactivation of sigH.

56. The modified bacterium of claim 55, wherein the dominant-negative mutant is a mutant SodA having a deletion of glutamic acid at position 54.

57. The modified bacterium of claim 55, wherein the dominant-negative mutant is a mutant SodA having deletions of histidine at position 28 and histidine at position 76.

58. The modified bacterium of claim 49, wherein the bacterium further comprises a dominant-negative mutant of SodA and inactivation of secA2.

59. The modified bacterium of claim 58, wherein the dominant-negative mutant is a mutant SodA having a deletion of glutamic acid at position 54.

60. The modified bacterium of claim 58, wherein the dominant-negative mutant is a mutant SodA having deletions of histidine at position 28 and histidine at position 76.

61. The modified bacterium of claim 2, wherein the bacterium comprises inactivation of sigH.

62. The modified bacterium of claim 2, wherein the bacterium comprises inactivation of sigH and inactivation of secA2.

63. A method of treating bladder cancer comprising administering pro-apoptotic BCG (paBCG) to a subject with bladder cancer.

64. The method of claim 63, wherein the administration is by instillation into the bladder.

65. A method of treating a solid tumor comprising administering paBCG to a subject with the solid tumor.

66. The method of claim 65, wherein the administration is by intralesional injection into the solid tumor.

67. The method of claim 65, wherein the administration is by intraarterial infusion into the artery that supplies the tumor.

68. The method of claim 65, wherein the solid tumor is selected from the group consisting of skin cancer, brain cancer, oropharyngeal cancer, breast cancer, lung cancer, esophageal cancer, stomach cancer, liver cancer, colon cancer, cancer of the biliary tract, pancreatic cancer, anal cancer, kidney cancer, prostate cancer, and sarcoma.

69. The method of claim 68, wherein
a) the skin cancer is melanoma or squamous cell carcinoma;
b) the brain cancer is glioblastoma, astrocytoma or oligodendroglioma;
c) the lung cancer is a primary tumor or metastasis of other tumors to lung; or
d) the liver cancer is a primary tumor (hepatoma) or metastasis of other tumors to the liver.

70. The method of claim 65, wherein the solid tumor is melanoma and the administration is by intralesional injection into the melanoma.

71. A method of treating cancer comprising administering to a subject with cancer an anti-cancer vaccine and paBCG.

72. The method of claim 71, wherein the anti-cancer vaccine and the paBCG are administered separately.

73. The method of claim 71, wherein the anti-cancer vaccine and the paBCG are administered separately and substantially concurrently.

74. The method of claim 71, wherein the anti-cancer vaccine and the paBCG are in a mixture.

75. A composition comprising an anti-cancer vaccine and paBCG.

76. The composition of claim 75, wherein the anti-cancer vaccine comprises a cancer antigen.

77. The composition of claim 75, wherein the anti-cancer vaccine comprises autologous cancer cells.

78. A composition comprising paBCG expressing dominant-negative mutant SodA, mutant SodA, or peptides of SodA and a pharmaceutically acceptable caner.

79. A method of preventing the development of active pulmonary tuberculosis comprising immunizing a subject with a composition comprising paBCG expressing dominant-negative mutant SodA, mutant SodA, or peptides of SodA.

80. A method of reducing lung damage in persons with active pulmonary tuberculosis comprising immunizing a subject with a composition comprising paBCG expressing dominant-negative mutant SodA, mutant SodA, or peptides of SodA.

81. A method of reducing lung fibrosis in persons infected by Mycobacterium species comprising immunizing of a subject with a composition comprising paBCG expressing dominant-negative mutant SodA, mutant SodA, or peptides of SodA.

82. A method of prolonging the survival of a subject with a cancer comprising administering paBCG to the subject.

83. A method of reducing the likelihood of cancer developing in a subject comprising administering paBCG to the subject.
